SEQUENCE LISTING (72 sequences)

SEQ ID NO: 1 - RNA, 64 nt, synthetic construct
  source 1..64: mol_type = other RNA
gatttagactaccccaaaaacgaaggggactaaaacttagttcctaggtactatacgttatgtc

SEQ ID NO: 2 - skipped

SEQ ID NO: 3 - DNA, 25 bp, synthetic construct
  source 1..25: mol_type = other DNA
  modified_base 25: mod_base = OTHER; note = 3' Hexanediol modified nucleotide
tgctgcagctagaagtccgagaagt

SEQ ID NO: 4 - DNA, 25 bp, synthetic construct
  source 1..25: mol_type = other DNA
  modified_base 25: mod_base = OTHER; note = 3' Hexanediol modified nucleotide
tgctgcagctagaagtccgtagaca

SEQ ID NO: 5 - DNA, 10 bp, synthetic construct
  source 1..10: mol_type = other DNA
  modified_base 1: mod_base = OTHER; note = 5' 6-Carboxyfluorescein modified nucleotide
  modified_base 10: mod_base = OTHER; note = 3' Black hole quencher-1 modified nucleotide
ttattttatt

SEQ ID NO: 6 - RNA, 41 nt, synthetic construct
  source 1..41: mol_type = other RNA
taatttctactaagtgtagatgaagtgatgacgagtgtcta

SEQ ID NO: 7 - DNA, 40 bp, synthetic construct
  source 1..40: mol_type = other DNA
tgaggaggaggaagtgatgacgagtgtctactcctcctca

SEQ ID NO: 8 - DNA, 26 bp, synthetic construct
  source 1..26: mol_type = other DNA
  modified_base 26: mod_base = OTHER; note = Nucleotide attached to 3' C3 Spacer
gaaggtcgaagatcgctgaggaggag

SEQ ID NO: 9 - DNA, 30 bp, synthetic construct
  source 1..30: mol_type = other DNA
  modified_base 30: mod_base = OTHER; note = Nucleotide attached to 3' C3 Spacer
taatacgactcactatagggccgagaagtg

SEQ ID NO: 10 - RNA, 56 nt, synthetic construct
  source 1..56: mol_type = other RNA
gatttagactaccccaaaaacgaaggggactaaaacagacactcgtcatcacttct

SEQ ID NO: 11 - DNA, 58 bp, synthetic construct
  source 1..58: mol_type = other DNA
ccatgcacgtgcgaagaagctataagacatgtacgtgcatggacttctagctgcagca

SEQ ID NO: 12 - DNA, 26 bp, synthetic construct
  source 1..26: mol_type = other DNA
  modified_base 26: mod_base = OTHER; note = 3' Hexanediol modified nucleotide
tgctgcagctagaagtccatgcacgt

SEQ ID NO: 13 - DNA, 60 bp, synthetic construct
  source 1..60: mol_type = other DNA
tagaagtccgaaacaaaataatttaagtttaatgttttcaaaaaattataaaagtcttgg

SEQ ID NO: 14 - DNA, 64 bp, synthetic construct
  source 1..64: mol_type = other DNA
tagaagtccagaaaatcacaagttcattattactcaaaaagaagcacttgctaactatatgcgg

SEQ ID NO: 15 - DNA, 60 bp, synthetic construct
  source 1..60: mol_type = other DNA
tagaagtccgaaacaaaataatttaagtttaatgtttccaaaaaattataaaagtcttgg

SEQ ID NO: 16 - DNA, 64 bp, synthetic construct
  source 1..64: mol_type = other DNA
tagaagtccagaaaatcacaagttcattattactccaaaagaagcacttgctaactatatgcgg

SEQ ID NO: 17 - DNA, 236 bp, synthetic construct
  source 1..236: mol_type = other DNA
tgaggaggagtaatacgactcactatagggcataagacaaatacgactctgtcagagagaattaagtaaggaggttttttatggattacaaagaccacgatggtgactacaaagaccatgatatagattacaaggatgatgatgataaatttttaagtgtgtcacttaacatttgtacatggtcacatcctcaattcgagaagtaataatctcctcctcagcgatcttcgaccttc

SEQ ID NO: 18 - DNA, 302 bp, synthetic construct
  source 1..302: mol_type = other DNA
tgaggaggagtaatacgactcactatagggcataagacaaatacgactctgtcagagagaattaagtaaggaggttttttatggattacaaagaccacgatggtgactacaaagaccatgatatagattacaaggatgatgatgataaatttttaagtgtgtcacttaacatttgtacaagtgcgtggtcacatcctcaattcgagaagggcggcgggagtggaggtggctccggtggatctgcttggtcacacccccaattcgaaaaataataatctcctcctcagcgatcttcgaccttc

SEQ ID NO: 19 - DNA, 194 bp, synthetic construct
  source 1..194: mol_type = other DNA
tgaggaggagtaatacgactcactatagggcataagacaaatacgactctgtcagagagaattaagtaaggaggttttttatggattacaaggatgatgatgataaatttttaagtgtgtcacttaacatttgtacatggtcacatcctcaattcgagaagtaataatctcctcctcagcgatcttcgaccttc

SEQ ID NO: 20 - DNA, 260 bp, synthetic construct
  source 1..260: mol_type = other DNA
tgaggaggagtaatacgactcactatagggcataagacaaatacgactctgtcagagagaattaagtaaggaggttttttatggattacaaggatgatgatgataaatttttaagtgtgtcacttaacatttgtacaagtgcgtggtcacatcctcaattcgagaagggcggcgggagtggaggtggctccggtggatctgcttggtcacacccccaattcgaaaaataataatctcctcctcagcgatcttcgaccttc

SEQ ID NO: 21 - DNA, 26 bp, synthetic construct
  source 1..26: mol_type = other DNA
gaaggtcgaagatcgctgaggaggag

SEQ ID NO: 22 - DNA, 18 bp, synthetic construct
  source 1..18: mol_type = other DNA
gggatcgctgaggaggag

SEQ ID NO: 23 - DNA, 26 bp, synthetic construct
  source 1..26: mol_type = other DNA
  modified_base 26: mod_base = OTHER; note = 3' Hexanediol modified nucleotide
gaaggtcgaagatcgctgaggaggag

SEQ ID NO: 24 - DNA, 43 bp, synthetic construct
  source 1..43: mol_type = other DNA
tgaggaggagtaatacgactcactataggggcacaatcaccaa

SEQ ID NO: 25 - DNA, 54 bp, synthetic construct
  source 1..54: mol_type = other DNA
  modified_base 1: mod_base = OTHER; note = 5' Phosphorylated nucleotide
aagatctgaatcgtggtcacatcctcaattcgagaagtaataatctcctcctca

SEQ ID NO: 26 - DNA, 117 bp, synthetic construct
  source 1..117: mol_type = other DNA
  modified_base 1: mod_base = OTHER; note = 5' Phosphorylated nucleotide
tcaaagttgaatctgcataaggaggttttttatggattacaaagaccacgatggtgactacaaagaccatgatatagattacaaggatgatgatgataaatcagagacaaagtcatt

SEQ ID NO: 27 - DNA, 67 bp, synthetic construct
  source 1..67: mol_type = other DNA
tgaggaggagtaatacgactcactataggggattaagtaaggaggttttttatggcacaatcaccaa

SEQ ID NO: 28 - DNA, 54 bp, synthetic construct; identical to SEQ ID NO: 25
  source 1..54: mol_type = other DNA
  modified_base 1: mod_base = OTHER; note = 5' Phosphorylated nucleotide

SEQ ID NO: 29 - DNA, 100 bp, synthetic construct
  source 1..100: mol_type = other DNA
  modified_base 1: mod_base = OTHER; note = 5' Phosphorylated nucleotide
tcaaagttgaatctgcagattacaaagaccacgatggtgactacaaagaccatgatatagattacaaggatgatgatgataaatcagagacaaagtcatt

SEQ ID NO: 30 - DNA, 69 bp, synthetic construct
  source 1..69: mol_type = other DNA
tgaggaggagtaatacgactcactataggggattaagtaaggaggttttttatgttattagctgtatgt

SEQ ID NO: 31 - DNA, 57 bp, synthetic construct
  source 1..57: mol_type = other DNA
  modified_base 1: mod_base = OTHER; note = 5' Phosphorylated nucleotide
cagcgtacccgtaggctggtcacatcctcaattcgagaagtaataatctcctcctca

SEQ ID NO: 32 - DNA, 95 bp, synthetic construct
  source 1..95: mol_type = other DNA
  modified_base 1: mod_base = OTHER; note = 5' Phosphorylated nucleotide
acagttgcacaatcagattacaaagaccacgatggtgactacaaagaccatgatatagattacaaggatgatgatgataaatctgaatcgacaag

SEQ ID NO: 33 - DNA, 80 bp, synthetic construct
  source 1..80: mol_type = other DNA
tgaggaggagtaatacgactcactataggggattaagtaaggaggttttttatgttattagctgtatgtacagttgcaca

SEQ ID NO: 34 - DNA, 66 bp, synthetic construct
  source 1..66: mol_type = other DNA
  modified_base 1: mod_base = OTHER; note = 5' Phosphorylated nucleotide
atcgacaagcagcgtacccgtaggctggtcacatcctcaattcgagaagtaataatctcctcctca

SEQ ID NO: 35 - DNA, 117 bp, synthetic construct
  source 1..117: mol_type = other DNA
  modified_base 1: mod_base = OTHER; note = 5' Phosphorylated nucleotide
atcaccaatcaaagttgaatctgcagattacaaagaccacgatggtgactacaaagaccatgatatagattacaaggatgatgatgataaatcagagacaaagtcattaagatctga

SEQ ID NO: 36 - DNA, 88 bp, synthetic construct
  source 1..88: mol_type = other DNA
tgaggaggagtaatacgactcactataggggattaagtaaggaggttttttatgttattagctgtatgtacagttgcacaatcaccaa

SEQ ID NO: 37 - DNA, 75 bp, synthetic construct
  source 1..75: mol_type = other DNA
  modified_base 1: mod_base = OTHER; note = 5' Phosphorylated nucleotide
aagatctgaatcgacaagcagcgtacccgtaggctggtcacatcctcaattcgagaagtaataatctcctcctca

SEQ ID NO: 38 - DNA, 100 bp, synthetic construct; identical to SEQ ID NO: 29
  source 1..100: mol_type = other DNA
  modified_base 1: mod_base = OTHER; note = 5' Phosphorylated nucleotide

SEQ ID NO: 39 - DNA, 58 bp, synthetic construct
  source 1..58: mol_type = other DNA
tgaggaggagatacagatcatggttgctttgtaggttacctgtaaaacctcctcctca

SEQ ID NO: 40 - DNA, 84 bp, synthetic construct
  source 1..84: mol_type = other DNA
tgaggaggagatgttcaacaatggggttttacaggtaacctacaaagcaaccatgatctgtattgtcaagtccactcctcctca

SEQ ID NO: 41 - DNA, 31 bp, synthetic construct
  source 1..31: mol_type = other DNA
  modified_base 31: mod_base = OTHER; note = 3' Hexanediol modified nucleotide
taatacgactcactatagggctgaggaggag

SEQ ID NO: 42 - DNA, 25 bp, synthetic construct
  source 1..25: mol_type = other DNA
tgaggaggagatgttcaacaatggg

SEQ ID NO: 43 - DNA, 22 bp, synthetic construct
  source 1..22: mol_type = other DNA
tgaggaggagatgttcaacaat

SEQ ID NO: 44 - DNA, 20 bp, synthetic construct
  source 1..20: mol_type = other DNA
tgaggaggagatgttcaaca

SEQ ID NO: 45 - DNA, 33 bp, synthetic construct
  source 1..33: mol_type = other DNA
ccgatcgctgaggaggagtggacttgacaatac

SEQ ID NO: 46 - DNA, 30 bp, synthetic construct
  source 1..30: mol_type = other DNA
ccgatcgctgaggaggagtggacttgacaa

SEQ ID NO: 47 - DNA, 28 bp, synthetic construct
  source 1..28: mol_type = other DNA
ccgatcgctgaggaggagtggacttgac

SEQ ID NO: 48 - DNA, 25 bp, synthetic construct
  source 1..25: mol_type = other DNA
tgaggaggagtggacttgacaatac

SEQ ID NO: 49 - DNA, 15 bp, synthetic construct
  source 1..15: mol_type = other DNA
attacgtctataatc

SEQ ID NO: 50 - DNA, 24 bp, synthetic construct
  source 1..24: mol_type = other DNA
tgaggaggagatacagatcatggt

SEQ ID NO: 51 - DNA, 10 bp, synthetic construct
  source 1..10: mol_type = other DNA
  modified_base 1: mod_base = OTHER; note = 5' Phosphorylated nucleotide
tgctttgtag

SEQ ID NO: 52 - DNA, 24 bp, synthetic construct
  source 1..24: mol_type = other DNA
  modified_base 1: mod_base = OTHER; note = 5' Phosphorylated nucleotide
gttacctgtaaaacctcctcctca

SEQ ID NO: 53 - DNA, 24 bp, synthetic construct
  source 1..24: mol_type = other DNA
tgaggaggagatacagatcatggt

SEQ ID NO: 54 - DNA, 12 bp, synthetic construct
  source 1..12: mol_type = other DNA
  modified_base 1: mod_base = OTHER; note = 5' Phosphorylated nucleotide
tgctttgtaggt

SEQ ID NO: 55 - DNA, 24 bp, synthetic construct
  source 1..24: mol_type = other DNA
  modified_base 1: mod_base = OTHER; note = 5' Phosphorylated nucleotide
tacctgtaaaacccctcctcctca

SEQ ID NO: 56 - DNA, 24 bp, synthetic construct
  source 1..24: mol_type = other DNA
tgaggaggaggacaatacagatca

SEQ ID NO: 57 - DNA, 12 bp, synthetic construct
  source 1..12: mol_type = other DNA
  modified_base 1: mod_base = OTHER; note = 5' Phosphorylated nucleotide
tggttgctttgt

SEQ ID NO: 58 - DNA, 24 bp, synthetic construct
  source 1..24: mol_type = other DNA
  modified_base 1: mod_base = OTHER; note = 5' Phosphorylated nucleotide
aggttacctgtaaactcctcctca

SEQ ID NO: 59 - DNA, 34 bp, synthetic construct
  source 1..34: mol_type = other DNA
tgaggaggagccatggacttgacaatacagatca

SEQ ID NO: 60 - DNA, 12 bp, synthetic construct
  source 1..12: mol_type = other DNA
  modified_base 1: mod_base = OTHER; note = 5' Phosphorylated nucleotide
tggttgctttgt

SEQ ID NO: 61 - DNA, 34 bp, synthetic construct
  source 1..34: mol_type = other DNA
  modified_base 1: mod_base = OTHER; note = 5' Phosphorylated nucleotide
aggttacctgtaaaaccccattgtctcctcctca

SEQ ID NO: 62 - RNA, 12 nt, synthetic construct
  source 1..12: mol_type = other RNA
  modified_base 1: mod_base = OTHER; note = 5' 6-Carboxyfluorescein modified nucleotide
  modified_base 12: mod_base = OTHER; note = 3' Black hole quencher-1 modified nucleotide
tttttttttttt

SEQ ID NO: 63-64 - skipped (2 entries)

SEQ ID NO: 65 - DNA, 122 bp, unidentified
  source 1..122: mol_type = unassigned DNA
ccgatcgctgaggaggagtggacttgacaatacagatcatggttgctttgtaggttacctgtaaaaccccattgttgaacatcaatcataaacggattatagacgtaatcaaatccaataga

SEQ ID NO: 66 - DNA, 20 bp, synthetic construct
  source 1..20: mol_type = other DNA
  modified_base 20: mod_base = OTHER; note = Nucleotide attached to 3'-Biotin-triethylene glycol spacer
tacctgtaaactcctcctca

SEQ ID NO: 67 - DNA, 20 bp, synthetic construct
  source 1..20: mol_type = other DNA
  modified_base 1: mod_base = OTHER; note = 5' 6-Carboxyfluorescein modified nucleotide
  modified_base 20: mod_base = OTHER; note = 3' Hexanediol modified nucleotide
atcatggttgctttgtaggt

SEQ ID NO: 68 - DNA, 60 bp, synthetic construct
  source 1..60: mol_type = other DNA
tgaggaggaggacaatacagatcatggttgctttgtaggttacctgtaaactcctcctca

SEQ ID NO: 69 - RNA, 64 nt, synthetic construct
  source 1..64: mol_type = other RNA
gatttagactaccccaaaaacgaaggggactaaaacgatcatggttgctttgtaggttacctgt

SEQ ID NO: 70 - DNA, 19 bp, synthetic construct
  source 1..19: mol_type = other DNA
taatacgactcactatagg

SEQ ID NO: 71 - DNA, 15 bp, synthetic construct
  source 1..15: mol_type = other DNA
tgtatgtatgtatga

SEQ ID NO: 72 - DNA, 25 bp, synthetic construct
  source 1..25: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  modified_base 25: mod_base = OTHER; note = Nucleotide attached to 3' Blocking molecule
nnnnnnnnntgtatgtatgtatgta

(19) United States
(12) Patent Application Publication
(10) Pub. No.: US 2026/0043073 A1
(43) Pub. Date: Feb. 12, 2026

(54) AMBIENT TEMPERATURE NUCLEIC ACID AMPLIFICATION AND DETECTION

(71) Applicant: Sherlock Biosciences, Inc., Watertown, MA (US)

(72) Inventors: Nicolaas Angenent-Mari, Somerville, MA (US); Nadish Goyal, Belmont, MA (US); Shichu Huang, Watertown, MA (US); Max Desmond, Cambridge, MA (US); Mary Katherine Wilson, Burlington, MA (US); Mary Elizabeth Natoli, Watertown, MA (US); Deborah Paola Almeida, West Newton, MA (US); Joycelynn Boakyewah Acheampong, Brighton, MA (US); Aric Joneja, Waltham, MA (US)

(21) Appl. No.: 18/877,160

(22) PCT Filed: Jun. 30, 2023
(86) PCT No.: PCT/US2023/026784

§ 371 Date: Dec. 19, 2024

Related U.S. Application Data
(60) Provisional application No. 63/358,065, filed on Jul. 1, 2022.

(51) Int. Cl. C12Q 1/6844; C12N 9/22; C12N 15/113; C12Q 1/48; C12Q 1/6816; C12Q 1/6876

(52) U.S. Cl. CPC C12Q 1/6844; C12N 9/226; C12N 15/113; C12Q 1/48; C12Q 1/6816; C12Q 1/6876; C12Y 207/07007; C12N 2310/122; C12N 2310/315; C12N 2310/33

(57) ABSTRACT

The present disclosure provides improved compositions and methods for amplification and detection of target nucleic acid(s) at ambient temperatures.

### CROSS REFERENCE TO RELATED APPLICATIONS

This application claims priority to U.S. Patent Application No. 63/358,065 filed Jul. 1, 2022 entire contents of which are hereby incorporated by reference.

### SEQUENCE LISTING

The present application contains a Sequence Listing which has been submitted electronically in XML format named “2013065-0653_generic_SL.xml” The XML file was created on Sep. 21, 2023, and is 84.9 kilobytes in size. The entire contents of the Sequence Listing are hereby incorporated by reference.

### BACKGROUND

Amplification and/or detection of nucleic acids in samples (e.g., biological and/or environmental samples) is increasingly important in a variety of diagnostic, therapeutic, social, and other contexts.

### SUMMARY

The present disclosure recognizes that present nucleic acid amplification methods and detection methods rely on higher than ambient temperatures often coupled with cycling temperatures. The present disclosure recognizes that relieving the need for temperatures above ambient temperature provides considerable advantages. The present disclosure recognizes that isothermal nucleic acid amplification at or near ambient temperatures allows for sensitive diagnostic methods performed in point-of-care settings. The present disclosure recognizes that isothermal nucleic acid amplification at or near ambient temperatures allows for sensitive diagnostic methods to be performed in resource limited settings.

The present disclosure provides certain technologies that permit amplification and detection of nucleic acids in samples (e.g., biological and/or environmental samples) at ambient temperature.

In some embodiments, compositions and methods disclosed herein result in robust amplification of a target nucleic acid. Robust amplification of a target nucleic acid sequence refers to compositions and methods that consistently amplify a target nucleic acid sequence to a detectable level. Technologies provided herein permit sensitive detection of nucleic acids of interest (i.e., nucleic acids whose nucleotide sequence is or includes a target sequence). In some embodiments, provided technologies are particularly useful or applicable for detection of low-abundance (e.g., less than about 10 fM, or about 1 fM, or about 100 aM) nucleic acids.

The present disclosure provides compositions comprising: (a) a double-stranded DNA target nucleic acid sequence comprising a first native restriction enzyme recognition sequence and a second native restriction enzyme recognition sequence on opposite strands separated by a target nucleic acid region; (b) a first SDA primer comprising: (i) a sequence complementary to the first native restriction enzyme recognition sequence; (ii) a 3′ blocking molecule; and (iii) a 5′ stabilization sequence comprising of about 8 to about 20 nucleotides; (c) a second SDA primer comprising: (i) a sequence complementary to the second restriction enzyme nickase recognition sequence; (ii) a 3′ blocking molecule; and (iii) a 5′ stabilization sequence comprising of about 8 to about 20 nucleotides; (d) a cleavage enzyme; (e) a DNA polymerase having strand displacement activity; (f) a single stranded binding protein; and (g) dNTPs.

The present disclosure provides methods of amplifying a double-stranded DNA target nucleic acid sequence comprising a first native restriction enzyme recognition sequence and a second native restriction enzyme recognition sequence on opposite strands separated by a target nucleic acid region in a sample, wherein the methods comprise: (A) contacting the sample with a composition comprising: (a) a first SDA primer comprising: (i) a sequence complementary to the first native restriction enzyme recognition sequence; (ii) a 3′ blocking molecule; and (iii) a 5′ stabilization sequence comprising of about 8 to about 20 nucleotides; (b) a second SDA primer comprising: (i) a sequence complementary to the second native restriction enzyme recognition sequence; (ii) a 3′ blocking molecule; and (iii) a 5′ stabilization sequence comprising about 8 to about 20 nucleotides; (c) a cleavage enzyme; (d) a DNA polymerase having strand displacement activity; (e) a single stranded binding protein; (f) dNTPs; and thereby generating a reaction mixture; (B) incubating the reaction mixture under conditions favorable for (i) the first and second SDA primers to hybridize to the double-stranded DNA target nucleic acid sequence in the sample; and (ii) amplification of the target nucleic acid, thereby generating multiple copies of an amplified target nucleic acid.

The present disclosure provides methods of detecting a double-stranded DNA target nucleic acid sequence comprising a first native restriction enzyme recognition sequence and a second native restriction enzyme recognition sequence on opposite strands separated by a target nucleic acid region in a sample, wherein the methods comprise: (A) contacting at least one copy of the amplified target nucleic acid sequence as described herein, with a composition comprising: (i) a guide nucleic acid comprising a nucleic acid sequence complementary to the DNA cassette; and (ii) a Cas enzyme with collateral cleavage activity; (iii) a nucleic acid reporter probe susceptible to the collateral cleavage activity, so that the nucleic acid reporter probe has a detectably different first uncleaved state and a second cleaved state; and (B) detecting cleavage of the nucleic acid reporter probe by detecting a difference between the first state and the second state to determine the presence of the target nucleic acid sequence in the sample.

The present disclosure provides methods of detecting a double-stranded DNA target nucleic acid sequence comprising a first native restriction enzyme recognition sequence and a second native restriction enzyme recognition sequence on opposite strands separated by a target nucleic acid region in a sample, wherein methods comprise: (A) contacting at least one copy of the amplified target nucleic acid sequence as described herein, with a composition comprising (i) a capture probe; and (ii) a conjugate capture probe comprising a detectable label and a 3′ blocking entity; (B) detecting presence of the detectable label bound to a solid substrate to determine the presence of the target nucleic acid sequence in the sample.

The present disclosure provides compositions comprising: (a) a double-stranded DNA target nucleic acid sequence comprising at least one native restriction enzyme recognition sequence; (b) a forward primer comprising: (i) a 3′ nucleic acid sequence complementary to a target nucleic acid sequence downstream of or 5′ to the native restriction enzyme recognition sequence; and (ii) a 5′ SDA primer binding sequence comprising a partial restriction enzyme recognition sequence; (c) a first SDA primer comprising (i) a sequence complementary to the at least one native restriction enzyme recognition sequence; (ii) a 3′ blocking molecule; and (iii) a 5′ stabilization sequence comprising of about 8 to about 20 nucleotides; (d) a second SDA primer comprising: (i) a sequence complementary to the forward primer 5′ SDA primer binding sequence comprising a partial restriction enzyme recognition sequence; (ii) a 3′ blocking molecule; and (iii) a 5′ stabilization sequence comprising about 8 to about 20 nucleotides, comprising a partial restriction enzyme recognition sequence that together with the partial restriction enzyme recognition sequence in the 5′ SDA primer binding sequence form a complete restriction enzyme recognition sequence; (e) a cleavage enzyme; (f) a DNA polymerase having strand displacement activity; (g) a single stranded binding protein; and (h) dNTPs.

The present disclosure provides methods of amplifying a double-stranded DNA target nucleic acid sequence comprising at least one native restriction enzyme sequence in a sample, wherein the method comprises (A) contacting the sample, with a composition comprising: (a) a forward primer comprising a 3′ nucleic acid sequence complementary to a target nucleic acid sequence downstream of or 5′ to the native nickase recognition sequence and a 5′ SDA primer binding sequence comprising a partial nickase recognition; (b) a cleavage enzyme; (c) a DNA polymerase having strand displacement activity; (d) a single stranded binding protein; (e) dNTPs; thereby generating a single stranded DNA (ssDNA) cassette; (B) contacting the ssDNA cassette of step (A) with a composition comprising: (f) a first SDA primer comprising: (i) a sequence complementary to the at least one native restriction enzyme sequence in the target nucleic acid sequence; (ii) a 3′ blocking molecule; and (iii) a stabilization sequence comprising about 8 to about 20 nucleotides, and (g) a second SDA primer comprising: (i) a sequence complementary to the forward primer 5′ SDA primer binding sequence comprising a partial restriction enzyme recognition sequence; (ii) a 3′ blocking molecule; and (iii) a 5′ stabilization sequence comprising about 8 to about 20 nucleotides, comprising a partial nickase recognition sequence that together with the partial nickase recognition sequence in the 5′ SDA primer binding sequence form a complete restriction enzyme recognition sequence, thereby producing a reaction mixture; (C) incubating the reaction mixture under conditions favorable for generation of multiple copies of nucleic acid identical or complementary to the ssDNA cassette.

The present disclosure provides methods of detecting a double-stranded DNA target nucleic acid sequence comprising at least one native restriction enzyme sequence in a sample, wherein the method comprises: (A) contacting at least one copy of the amplified target nucleic acid sequence as described herein, with a composition comprising: (i) a guide nucleic acid comprising a nucleic acid sequence complementary to the DNA cassette; and (ii) a Cas enzyme with collateral cleavage activity; (iii) a nucleic acid reporter probe susceptible to the collateral cleavage activity, so that the nucleic acid reporter probe has a detectably different first uncleaved state and a second cleaved state; and (B) detecting cleavage of the nucleic acid reporter probe by detecting a difference between the first state and the second state to determine the presence of the target nucleic acid sequence in the sample.

The present disclosure provides methods of detecting a double-stranded DNA target nucleic acid sequence comprising at least one native restriction enzyme sequence in a sample, wherein the method comprises: (A) contacting at least one copy of the amplified target nucleic acid sequence as described herein, with a composition comprising: (i) a capture probe; and (ii) a conjugate capture probe comprising a detectable label and a 3′ blocking entity; (B) detecting presence of the detectable label bound to a solid substrate to determine the presence of the target nucleic acid sequence in the sample.

The present disclosure provides a composition comprising: (a) a RNA target nucleic acid; (b) a reverse primer comprising: (i) a 3′ sequence complementary to the RNA target nucleic acid; and (ii) a 5′ SDA primer binding sequence, wherein the primer binding sequence comprises a restriction enzyme recognition sequence, a partial restriction enzyme recognition sequence, or complements thereof, (c) a reverse transcriptase; and (d) a forward primer comprising: (i) a 3′ sequence complementary to the RNA target polynucleotide; and (ii) a 5′ SDA primer binding, wherein the primer binding sequence comprises a restriction enzyme recognition sequence or a partial restriction enzyme recognition sequence, or complements thereof, (e) a first SDA primer comprising: (i) a sequence complementary to the 5′ SDA primer binding sequence of the reverse primer; (ii) a 3′ blocking molecule; and (iii) a 5′ stabilization sequence comprising of about 8 to about 20 nucleotides, and when the reverse primer comprises only a partial restriction enzyme recognition sequence the first SDA primer comprises a partial restriction enzyme recognition sequence that together with the partial restriction enzyme recognition sequence in the 5′ SDA primer binding sequence of the reverse primer form a complete restriction enzyme recognition sequence; (f) a second SDA primer comprising: (i) a sequence complementary to the 5′ SDA primer binding sequence of the forward primer; (ii) a 3′ blocking molecule; and (iii) a 5′ stabilization sequence comprising of about 8 to about 20 nucleotides, when the forward primer comprises only a partial restriction enzyme recognition sequence the first SDA primer comprises a partial restriction enzyme recognition sequence that together with the partial restriction enzyme recognition sequence in the 5′ SDA primer binding sequence of the forward primer form a complete restriction enzyme recognition sequence; (g) a cleavage enzyme; (h) a polymerase having strand displacement activity; (i) a single stranded binding protein; and (j) dNTPs.

The present disclosure provides methods of amplifying a RNA target nucleic acid sequence in a sample comprising: (A) contacting the sample, with a composition comprising (a) a reverse primer comprising: (i) a 3′ sequence complementary the RNA target nucleic acid; and (ii) a 5′ SDA primer binding sequence, wherein the primer binding sequence comprises a restriction enzyme recognition sequence, a partial restriction enzyme recognition sequence, or complements thereof; (b) a reverse transcriptase; and (c) a forward primer comprising: (i) a 3′ sequence complementary the RNA target nucleic acid; and (ii) a 5′ SDA primer binding sequence, wherein the primer binding sequence comprises a restriction enzyme recognition sequence, a partial restriction enzyme recognition sequence, or complements thereof, thereby producing a first reaction mixture; (B) incubating the first reaction mixture under conditions favorable for generation of a single stranded DNA (ssDNA) cassette; (C) contacting the ssDNA cassette of (B) with a composition comprising: (d) a first SDA primer comprising: (i) a sequence complementary to the 5′ SDA primer binding sequence of the reverse primer; (ii) a 3′ blocking molecule; and (iii) a 5′ stabilization sequence comprising about 8 to about 20 nucleotides, and when the reverse primer comprises only a partial restriction enzyme recognition sequence the first SDA primer comprises a partial restriction enzyme recognition sequence that together with the partial restriction enzyme recognition sequence in the 5′ SDA primer binding sequence of the reverse primer form a complete restriction enzyme recognition sequence; (e) a second SDA primer comprising: (i) a sequence complementary to the 5′ SDA primer binding sequence of the forward primer; (ii) a 3′ blocking molecule; and (iii) a 5′ stabilization sequence comprising of about 8 to about 20 nucleotides, when the forward primer comprises only a partial restriction enzyme recognition sequence the first SDA primer comprises a partial restriction enzyme recognition sequence that together with the partial restriction enzyme recognition sequence in the 5′ SDA primer binding sequence of the forward primer form a complete restriction enzyme recognition sequence; and (f) at least one cleavage enzyme, polymerase having strand displacement activity, single stranded binding protein, dNTPs; and thereby producing a second reaction mixture; (D) incubating the second reaction mixture under conditions favorable for generation of multiple copies of a nucleic acid identical or complementary to the ssDNA cassette.

The present disclosure provides methods of detecting a RNA target nucleic acid sequence in a sample comprising, wherein the method comprises: (A) contacting at least one copy of the nucleic acid identical or complementary to the ssDNA cassette as described herein, with a composition comprising: (i) a guide nucleic acid comprising a nucleic acid sequence complementary to the DNA cassette; and (ii) a Cas enzyme with collateral cleavage activity; (iii) a nucleic acid reporter probe susceptible to the collateral cleavage activity, so that the nucleic acid reporter probe has a detectably different first uncleaved state and a second cleaved state; and (B) detecting cleavage of the nucleic acid reporter probe by detecting a difference between the first state and the second state to determine the presence of the RNA target nucleic acid sequence in the sample.

The present disclosure provides methods of detecting a RNA target nucleic acid sequence in a sample comprising, wherein the method comprises: (A) contacting at least one copy of the amplified RNA target nucleic acid sequence as described herein, with a composition comprising (i) a capture probe; and (ii) a conjugate capture probe comprising a detectable label and a 3′ blocking entity; (B) detecting presence of the detectable label bound to a solid substrate to determine the presence of the RNA target nucleic acid sequence in the sample.

The present disclosure provides compositions comprising: (a) a target polynucleotide; (b) a first probe comprising a 3′ SDA primer binding sequence comprising a partial nickase recognition site or complement thereof, a nucleic acid sequence that is complementary to the target nucleic acid, and a first nucleic acid sensory part; (c) a second probe comprising a 5′ SDA primer binding sequence comprising a partial nickase recognition site or complement thereof, a nucleic acid sequence that is complementary to the target nucleic acid, and second nucleic acid sensor part, wherein each nucleic acid sensory part comprises a sequence that is, encodes, or templates coding of a first and second reporting element component, respectively: (d) at least one gap filling oligo; and (e) a ligase, when the first and second probe are ligated together, generate a single strand of a DNA cassette that encodes at least one reporter and comprises a 3′ SDA primer binding sequence and a 5′ SDA primer binding sequence, wherein the SDA primer binding sequences comprise a partial nickase recognition sequence or a complement thereof.

The present disclosure provides compositions comprising: (a) a single stranded DNA cassette that encodes at least one reporter and comprises a 3′ SDA primer binding sequence and a 5′ SDA primer binding site, wherein the 3′ and 5′ SDA primer binding sequences comprise a partial nickase recognition sequence or complements thereof, (b) a first SDA primer comprising: (i) a sequence complementary to the 3′ SDA primer binding sequence; (ii) a 3′ blocking molecule; and (iii) a 5′ stabilization sequence comprising about 8 to about 20 nucleotides, comprising a partial nickase recognition sequence that together with the partial nickase recognition sequence in the 3′ SDA primer binding sequence form a complete nickase primer binding sequence; (c) a second SDA primer comprising: (i) a sequence complementary to the 5′ SDA primer binding sequences; (ii) a 3′ blocking molecule; and (iii) a 5′ stabilization sequence comprising about 8 to about 20 nucleotides, comprising a partial nickase recognition site that together with the partial nickase recognition sequence in the 5′ SDA primer binding sequence form a complete nickase primer binding sequence; and (d) at least one, nickase, polymerase having strand displacement activity, single stranded binding protein and dNTPs.

The present disclosure provides a composition comprising: (a) a single stranded DNA (ssDNA) cassette that encodes at least one reporter and comprises a 3′ SDA primer binding sequence and a 5′ SDA primer binding sequence, wherein the 3′ and 5′ SDA primer binding sequences comprise a partial nickase recognition sequence or a complement thereof, (b) a first SDA primer comprising (i) a sequence complementary to the 3′ SDA primer binding sequence; (ii) a 3′ blocking molecule; and (iii) a 5′ stabilization sequence comprising about 8 to about 20 nucleotides, comprising a partial nickase recognition site that together with the partial nickase recognition sequence in the 3′ SDA primer binding sequence form a complete nickase primer binding sequence; (c) a second SDA primer comprising: (i) a sequence complementary to the 5′ SDA primer binding sequences; (ii) a 3′ blocking molecule; and (iii) a 5′ stabilization sequence comprising about 8 to about 20 nucleotides, comprising a partial nickase recognition site that together with the partial nickase recognition sequence in the 5′ SDA primer binding sequence form a complete nickase primer binding sequence; and (d) a detection system comprising: (i) a guide nucleic acid comprising a nucleic acid sequence complementary to the DNA cassette; and (ii) a Cas enzyme with collateral cleavage activity; (iii) a nucleic acid reporter probe susceptible to the collateral cleavage activity, so that the nucleic acid reporter probe has a detectably different first uncleaved state and a second cleaved state; and (e) at least one nickase, polymerase having strand displacement activity, single stranded binding protein and dNTPs.

The present disclosure provides methods of detecting a target nucleic acid sequence in a sample comprising: (A) contacting the sample with a composition comprising: (a) a first probe comprising: a 3′ SDA primer binding sequence comprising a partial nickase recognition sequence or complement thereof, a nucleic acid sequence that is complementary to the target nucleic acid, and a first nucleic acid sensory part; (b) a second probe comprising: a 5′ SDA primer binding sequence comprising a partial nickase recognition sequence or complement thereof, a nucleic acid sequence that is complementary to the target nucleic acid, and second nucleic acid sensor part, wherein each nucleic acid sensory part comprises a sequence that is, encodes, or templates coding of a first and second reporting element component, respectively: (c) at least one gap filling oligo; and (d) a ligase, wherein, when the first and second probe are ligated together, generate a single strand of a DNA cassette that encodes at least one reporter and comprises a 3′ SDA primer binding sequence and a 5′ SDA primer binding sequence, wherein the SDA primer binding sequences comprise a partial nickase recognition sequence or a complement thereof, thereby producing a first reaction mixture; (B) incubating the first reaction mixture under conditions favorable for generation of a single stranded DNA (ssDNA) cassette, (C) contacting the ssDNA cassette of (B) with a composition comprising: (i) a first SDA primer comprising: a sequence complementary to the 3′ SDA primer binding sequence; a 3′ blocking molecule; and a stabilization sequence comprising about 8 to about 20 nucleotides, comprising a partial nickase recognition site that together with the partial nickase recognition sequence in the 3′ SDA primer binding sequence form a complete nickase primer binding sequence; (ii) a second SDA primer comprising: a sequence complementary to the 5′ SDA primer binding sequences; a 3′ blocking molecule; and a 5′ stabilization sequence comprising about 8 to about 20 nucleotides, comprising a partial nickase recognition site that together with the partial nickase recognition sequence in the 5′ SDA primer binding sequence form a complete nickase primer binding sequence; and (iii) at least one nickase, polymerase having strand displacement activity, and single stranded binding protein; thereby producing a second reaction mixture; (D) incubating the second reaction mixture under conditions favorable for generation of multiple copies of a nucleic acid identical or complementary to the ssDNA cassette; (E) contacting at least one copy of a nucleic acid identical or complementary to the ssDNA cassette with a composition comprising a cell free extract thereby producing a third reaction mixture; (F) incubating the third reaction mixture under conditions favorable for generation of the at least one reporter encoded by the ssDNA cassette; and (G) measuring the expression of the reporter protein produced in step (F) to determine the presence and/or amount of the target nucleic acid sequence in the sample.

The present disclosure provides a composition comprising: (a) a single stranded DNA cassette that encodes at least one reporter and comprises a 3′ SDA primer binding sequence and a 5′ SDA primer binding sequence, wherein the 3′ and 5′ SDA primer binding sequences comprise a nickase recognition sequence or a partial nickase recognition sequence, or complements thereof, (b) a first SDA primer comprising: (i) a sequence complementary to the 3′ SDA primer binding sequences: (ii) a 3′ blocking molecule; and (iii) a T7 promoter sequence, and/or a stabilization sequence comprising about 8 to about 20 nucleotides, comprising a partial nickase recognition site that together with the partial nickase recognition site on the 3′ SDA primer binding sequence forms a complete nickase primer binding sequence; (c) a second SDA primer comprising: (i) a sequence complementary to the 5′ SDA primer binding sequences; (ii) a 3′ blocking molecule; and (iii) a 5′ stabilization sequence comprising about 8 to about 20 nucleotides, comprising a partial nickase recognition site that together with the partial nickase recognition sequence in the 5′ SDA primer binding sequence form a complete nickase primer binding sequence; and (d) a detection system comprising: (i) a capture probe; and (ii) a conjugate capture probe comprising a detectable label and a 3′ blocking molecule; and (e) at least one nickase, polymerase having strand displacement activity, single stranded binding protein and dNTPs.

The present disclosure provides methods of detecting a RNA target nucleic acid sequence in a sample comprising: (A) contacting the sample, with a composition comprising: a reverse primer comprising: (i) a 3′ sequence complementary the RNA target nucleic acid; and (ii) a 5′ SDA primer binding sequence, wherein the primer binding sequence comprises a nickase recognition sequence, or a partial nickase recognition sequence or complements thereof, a reverse transcriptase, a forward primer comprising: (i) a 3′ sequence complementary the RNA target polynucleotide; and (ii) a 5′ SDA primer binding sequence, wherein the primer binding sequence comprises a nickase recognition sequence or a partial nickase recognition sequence, or a complement thereof, thereby producing a first reaction mixture; (B) incubating the first reaction mixture under conditions favorable for generation of a single stranded DNA (ssDNA) cassette; (C) contacting the ssDNA cassette of (B) with a composition comprising: (i) a first SDA primer comprising: a sequence complementary to the 5′ SDA primer binding sequence; a 3′ blocking molecule; and a T7 promoter sequence, and/or a stabilization sequence comprising or consisting of at about 8 to about 20 nucleotides, and when the 5′ SDA primer binding sequence only comprises a partial nickase recognition site the stabilization sequence comprises a partial nickase recognition site that together with the partial nickase recognition site on the SDA primer binding sequence form a complete nickase primer binding site; (ii) a second SDA primer comprising: a sequence complementary to the 3′ SDA primer binding sequence of the reverse primer; a 3′ blocking molecule; and a stabilization sequence comprising or consists of at least 8-12 nucleotides, and when the 3′ SDA primer binding sequence only comprises a partial nickase recognition site the stabilization sequence comprises a partial nickase recognition site that together with the partial nickase recognition site on the 3′ SDA primer binding sequence form a complete nickase primer binding site; and (ii) at least one nickase, polymerase having strand displacement activity, and single stranded binding protein, thereby producing a second reaction mixture; (D) incubating the second reaction mixture under conditions favorable for generation of multiple copies of a nucleic acid identical or complementary to the ssDNA cassette; (E) contacting at least one copy of the nucleic acid identical or complementary to the ssDNA cassette with a composition comprising (i) a capture probe; and (ii) a conjugate capture probe comprising a detectable label and a 3′ blocking molecule; (f) detecting presence of the detectable label bound to a solid substrate to determine the presence of the target nucleic acid sequence in the sample.

The present disclosure provides methods of detecting a target nucleic acid sequence in a sample comprising: (a) contacting the sample with a composition comprising (i) a first probe comprising a 3′ SDA primer binding sequence comprising a partial nickase recognition site or complement thereof, a nucleic acid sequence that is complementary to the target nucleic acid, and a first nucleic acid sensor part; (ii) a second probe comprising a 5′ SDA primer binding sequence comprising a partial nickase recognition site or complement thereof, a nucleic acid sequence that is complementary to the target nucleic acid, and second nucleic acid sensor part, wherein each nucleic acid sensory part comprises a sequence that is, encodes, or templates coding of a first and second reporting element component, respectively; (iii) at least one gap filling oligo, wherein; and (iv) a ligase, when ligated together, generate a single strand of a nucleotide cassette that encodes at least one reporter and comprises a 3′ SDA primer binding sequence and a 5′ SDA primer binding sequence, wherein the primer binding sequence comprises a nickase recognition sequence or a partial nickase recognition sequence thereby producing a first reaction mixture; (b) incubating the first reaction mixture under conditions favorable for generation of a single stranded nucleotide cassette, (c) contacting the nucleotide cassette of (b) with a composition comprising: (i) a first SDA primer comprising: (a) a sequence complementary to the 3′ SDA primer binding sequence; (b) a 3′ blocking molecule; and (c) a stabilization sequence comprising about 8 to about 20 nucleotides and comprising a partial nickase recognition site that together with the partial nickase recognition site on the 3′ SDA primer binding sequence form a complete nickase primer binding site; (c) a second SDA primer comprising: (i) a sequence complementary to the 5′ SDA primer binding sequences; (ii) a 3′ blocking molecule; and (iii) a 5′ stabilization sequence comprising about 8 to about 20 nucleotides, comprising a partial nickase recognition site that together with the partial nickase recognition sequence in the 5′ SDA primer binding sequence form a complete nickase primer binding sequence; and (ii) at least one nickase, polymerase having strand displacement activity, and single stranded binding protein; thereby producing a second reaction mixture; (d) incubating the second reaction mixture under conditions favorable for generation of multiple copies of a nucleic acid identical or complementary to the nucleotide cassette; (e) contacting at least one copy of a nucleic acid identical or complementary to the nucleotide cassette with a composition comprising (i) a guide nucleic acid comprising a nucleic acid sequence complementary to the nucleotide cassette; and (ii) a Cas enzyme with collateral cleavage activity; (iii) a nucleic acid reporter probe susceptible to the collateral cleavage activity, so that the nucleic acid reporter probe has a detectably different first uncleaved state and a second cleaved state; (f) detecting cleavage of the nucleic acid reporter probe by detecting a difference between the first state and the second state to determine the presence of the target nucleic acid sequence in the sample.

### BRIEF DESCRIPTION OF THE DRAWINGS

FIG. 1. Amplification of Dual-Epitope cassette (Mock Trigger, Seq 1) with different SDA Primers. S158 ROI: a specific target region of interest (ROI) in the SARS genome. 3xF 1xS: expression cassette comprising an open reading frames for three FLAG epitope tags (3xF) followed by one StrepII epitope tag (1xS). High/Low Bsu: High concentration or Low concentration Bsu DNA polymerase products.

FIG. 2. Amplification of Dual-Epitope cassettes of different lengths.

FIG. 3. Amplification of Dual-Epitope cassettes of different lengths.

FIG. 4. Lateral Flow LOD of a cassette using Cap-SDA.

FIG. 5. Reduction of NTC background signal from a ligation reaction amplified by Cap-SDA using ET-SSB.

FIG. 6. Reduction of NTC background signal from a ligation reaction amplified by Cap-SDA with new probe design. ROI 20945: region of interest (ROI) in the SARS genome. The probe design had an A probe, followed by a GFO, followed by a B probe. Old Design: the ribosome binding site (RBS) was located on the GFO (gap filling oligo). New Design: the ribosome binding site (RBS) was located on one of the A probe.

FIG. 7. Detection of SARS gRNA target in a ligation reaction amplified by Cap-SDA with different probe design. Three different probes were used: “30 bp Standard” probes having two 30 bp hybridization regions and no gap between them, “40 bp Gap” probes having two 30 bp hybridization regions and a 40 bp gap between them, and “51 bp HybSeqs” probes having two 51 bp hybridization regions and no gap between them, that were either of unequal length on either probe arm, or of equal length on either probe arm. ROI S20945: a region of interest (ROI) in the SARS genome.

FIG. 8. Detection of synthetic ssDNA targets containing ORF1 SARS sequence. HC: High concentration of DNA polymerase. LC: Low concentration of DNA polymerase.

FIG. 9. Detection of synthetic ssDNA targets containing ORF1 SARS sequence. KF-Treated: a reaction with SDA primers that were pre-treated with the 3′ exonuclease activity of the Klenow Large Fragment DNA polymerase before being added to the reaction.

FIG. 10. Detection of SARS irradiated viral particles using either a Reverse Transcriptase (left) or a Ligase reaction (right), amplified subsequently by Cap-SDA. BET: inactivated SARS viral material.

FIG. 11. Detection of SARS gRNA using a Reverse Transcriptase, amplified subsequently by Cap-SDA.

FIG. 12. Detection of SARS gRNA using a Reverse Transcriptase, amplified subsequently by Cap-SDA.

FIG. 13. Detection of SARS gRNA using a Reverse Transcriptase, amplified subsequently by Cap-SDA.

FIG. 14. Detection of SARS gRNA using a Ligase reaction, amplified subsequently by Cap-SDA.

FIG. 15. Detection of 2 fM of ssDNA synthetic target containing the SARS ORF1 sequence by Cap-SDA.

FIG. 16. Colorimetric detection of SARS gRNA at indicated concentrations using a Ligase reaction amplified subsequently by Cap-SDA.

FIG. 17. Detection of a dsDNA target containing a native nickase site in a one-pot SDA reaction.

FIG. 18. Detection of an ssDNA synthetic target (Seq 52, − indicates 0 fM, + indicates 20 fM) with lateral flow output form a Cap-SDA reaction using capture oligos.

FIG. 19. Detection of an ssDNA synthetic target (2 fM, Seq 52) with a Cap-SDA reaction under standard or pH-adjusted conditions.

FIG. 20 shows bridged ligation and CAP-SDA reaction to detect a target nucleic acid. The product of the SDA is a DNA encoding one or more reporters. The reporters can be generated using a cell free extract (CFE) and detected by lateral flow (LF). Exemplary assay steps include 30 min Ligation, 90 min SDA, 120 min CFE, 20 min LF and an approximate limit of detection (LOD) of about 1 fM

FIGS. 21A-21D show bridged ligation, CAP-SDA reaction, and CRISPR/Cas based detection (e.g., SHERLOCK™) to detect a target nucleic acid. The product of the SDA is a DNA comprising T7 promoter. A T7 RNA polymerase transcribes an RNA. The RNA is recognized by a Cas enzyme (e.g. as described in PCT/US2017/065477).

FIGS. 22A-22F show reverse transcription, CAP-SDA reaction, and CRISPR/Cas based detection (e.g., SHERLOCK™) to detect a target nucleic acid. The product of the SDA is a DNA comprising T7 promoter. A T7 RNA polymerase transcribes an RNA. The RNA is recognized by a CRISPR/Cas enzyme (e.g. as described in PCT/US2017/065477).

FIG. 23 show reverse transcription, CAP-SDA reaction, and, a capture lateral biosensor to detect a target nucleic acid. The product of the SDA is a DNA comprising T7 promoter. A T7 RNA polymerase transcribes an RNA. The RNA is recognized by binding to a capture probe and a conjugate capture probe (e.g., on a lateral flow biosensor).

FIG. 24A-24D shows dsDNA detection using a CAP-SDA reaction, and CRISPR/Cas based detection (e.g., SHERLOCK™) to detect a target nucleic acid. A T7 RNA polymerase transcribes an RNA. The RNA is recognized by a CRISPR/Cas enzyme (e.g. as described in PCT/US2017/065477).

FIG. 25A-25C shows dsDNA detection using a CAP-SDA reaction when the dsDNA comprises an endogenous nickase sequence.

FIG. 26 shows an exemplary workflow for viral lysis at ambient temperature (e.g., at 22° C.), using e.g., 3-Dodecylamido-N,N′-Dimethylpropyl Amine Oxide·3-Laurylamido-N,N′-Dimethylpropyl Amine Oxide (LAPAO) and hydrochloric acid (HCl), or using N,N-Dimethyl-1-Dodecanamine-N-Oxide (LDAO), sodium decanoate (NaCl10), and HCl. The workflow includes an incubation step of the sample with a lysis reagent, followed by lysis reaction and a neutralization step. The workflow also depicts an optional nucleic acid ligation step.

FIG. 27 shows effect(s) of pH on viral lysis efficiency at ambient temperature (22° C.) using detergent compositions as provided herein.

FIG. 28. Detection of a dsDNA genomic target (Ct extracted genome) using a Nb.BbvCI endogenous nick site with a Cap-SDA reaction without the use of a bump primer.

FIG. 29. Detection of a dsDNA genomic target (Ng extracted genome) with a Cap-SDA reaction with the nickase Nt.CviPII and LwCas13a.

FIG. 30. Detection of a dsDNA genomic target (Ng extracted genome) with a Cap-SDA reaction with the nickase Nt.CviPII and LbCas12a.

FIG. 31. Detection of a dsDNA genomic target (Ng extracted genome) with a Cap-SDA reaction with the nickase Nt.CviPII.

FIG. 32. Multiplexed detection of two dsDNA genomic targets (either Ct or Ng extracted genome) with a Cap-SDA reaction with the nickase Nt.CviPII.

FIG. 33. Detection of a dsDNA genomic target (Ng extracted genome) with a Cap-SDA reaction with the nickase Nt.CviPII.

FIG. 34A-34B. Detection of a synthetic ssDNA target (Seq A) with a Cap-SDA reaction with primer stabilizers that are either single stranded or a double stranded hairpin.

FIG. 35. Detection of a dsDNA genomic target (Ng extracted genome) with a Cap-SDA reaction with primer stabilizers that are either single stranded or a double stranded hairpin.

FIG. 36. Detection of a synthetic ssDNA target (Seq X) with a Cap-SDA reaction with the nickase Nt.CviPII.

FIG. 37. Detection of synthetic ssDNA targets (Seq X, Y, Z, and W) with a Cap-SDA reaction with the nickase Nt.CviPII.

FIGS. 38A-D show reverse transcription and CAP-SDA reaction of a target nucleic acid.

FIGS. 39-AB show exemplary SDA primers. Bold indicates a restriction enzyme recognition sequence, such as a nickase recognition sequence. A. * represents a nickase sequence blocked with PTO bonds. B. show exemplary SDA primers having a hairpin stabilization sequence. * represents a PTO bond. BLCK: 3′ blocking molecule.

FIG. 40 shows detection of amplicon capture probes (SEQ ID NO: 14, 15, 39) on a lateral flow without and with blocking molecules (10% skim milk or 1 uM Sponge Oligo).

FIG. 41A-41B shows lysis of respiratory viruses. A) InfluenzaA (FLUA) and B) SARS-CoV-2 (SCV2) using 95° C. heat lysis or 20 mM NaOH versus no treatment in individual nasal swab matrices. Viral lysis efficiency was assayed by first performing a room temperature reverse transcriptase reactions, followed by the inactivation of the reverse transcriptase and standard taqman qPCR of the produced cDNA. A lower Cq value corresponds to greater viral RNA release.

FIG. 42 shows RNase inhibition achieved in nasal swab matrix using an RNase inhibitor or Sodium hydroxide (NaOH). RNase activity was measured using the RNase Alert reagent from IDT.

FIG. 43 shows lysis of respiratory viruses (FluA and SCV2) with high pH solutions. Various concentrations of KOH and NaOH were used to lyse viral particles. Viral lysis efficiency was assayed by first performing a room temperature reverse transcriptase reactions, followed by the inactivation of the reverse transcriptase and standard taqman qPCR of the produced cDNA. A lower Cq value corresponds to greater viral RNA release, and therefore better lysis of the viral particle

FIG. 44 shows hydroxide-based chemical lysis of a non-enveloped virus. A stock of Human adevnovirus was treated the indicated concentration of NaOH at room temperature prior to qPCR to detect released viral DNA. A faster Cq value indicates a higher concentration of released viral DNA, and therefore better lysis of the viral particle.

FIG. 45A-45B shows room temperature lysis of bacteria A) N. gonorrhoeae and B) C. trachomatis. Bacterial cells were treated with the indicated concentration of KOH or subjected to bead beating. After lysis, the cell suspensions were centrifuged to pellet intact cells, and a portion of the remaining supernatant was assayed by qPCR. Results are presented as the difference in Cq values between the treated cells and the untreated control, with a larger delta indicating a more effective lysis.

FIG. 46 shows lysis of bacteria N. gonorrhoeae with 50 mM KOH with the addition of a detergent. Bacterial cells were treated with the indicated concentration of detergent in the presence of 50 mM KOH, or subjected to bead beating. After lysis, the cell suspensions were centrifuged to pellet intact cells, and a portion of the remaining supernatant was assayed by qPCR. Results are presented as the difference in Cq values between the treated cells and the untreated control, with a larger delta indicating a more effective lysis.

FIG. 47 shows lysis efficiency of N. gonorrhoeae treated with 50 mM KOH, 13.5 mM HCL+/−0.5% Pluronic 64 detergent at various incubation temperatures. Bacterial cells were treated with the indicated concentration of KOH, HCl, and/or detergent at the indicated temperature for 5 minutes, or subjected to a heat lysis of 95 C for 5 minutes. After lysis, a portion of the remaining supernatant was assayed by qPCR, with the concentration of gDNA in the samples quantified by a standard curve of extracted gDNA. Results are presented as the percent efficiency of lysis, assuming 100% lysis for the heat lysis control. The signal from untreated control cells (“no lysis”) was subtracted from all samples.

FIG. 48 shows lysis efficiency testing of N. gonorrhoeae treated with 50 mM KOH or HCL+ESH9 at various incubation temperatures. Bacterial cells were treated with the indicated concentration of KOH, HCl, and/or detergent at the indicated temperature for 5 minutes, or subjected to a heat lysis of 95 C for 5 minutes. After lysis, a portion of the remaining supernatant was assayed by qPCR, with the concentration of gDNA in the samples quantified by a standard curve of extracted gDNA. Results are presented as the percent efficiency of lysis, assuming 100% lysis for the heat lysis control. The signal from untreated control cells (“no lysis”) was subtracted from all samples.

FIG. 49 shows lysis efficiency testing of N. gonorrhoeae treated with 50 mM KOH, +NP40 at various incubation temperatures. Bacterial cells were treated with the indicated concentration of KOH, HCl, and/or detergent at the indicated temperature for 5 minutes, or subjected to a heat lysis of 95 C for 5 minutes. After lysis, the cell suspensions were centrifuged to pellet intact cells, and a portion of the remaining supernatant was assayed by qPCR. Results are presented as the percent efficiency of lysis, assuming 100% lysis for the heat lysis control.

FIG. 50 shows lysis efficiency testing of N. gonorrhoeae treated with various KOH concentrations, +/−3% NP40 at various incubation temperatures. Bacterial cells were treated with the indicated concentration of KOH and/or detergent at the indicated temperature for 5 minutes, or subjected to a heat lysis of 95 C for 5 minutes. After lysis, a portion of the remaining supernatant was assayed by qPCR, with the concentration of gDNA in the samples quantified by a standard curve of extracted gDNA. Results are presented as the percent efficiency of lysis, assuming 100% lysis for the heat lysis control. The signal from untreated control cells (“no lysis”) was subtracted from all samples.

FIG. 51 shows lysis technologies as described herein versus Heat lysis methods (95° C.) upstream of LAMP-Cas detection. N. gonorrhoeae were diluted in TE, treated as indicated, and a portion of the lysate was used as template for a LAMP-Cas reaction.

FIG. 52 shows KOH lysis of N. gonorrhoeae in vaginal matrix using LAMP-Cas. N. gonorrhoeae were diluted in TE, treated as indicated, and a portion of the lysate was used as template for a LAMP-Cas reaction.

FIG. 53 shows a nickase based CapSDA workflow. Bold nucleotides represent a nickase recognition sequence. SDA primer 1 and 2 are shown as hairpin primers, but can also be single-stranded sequences.

FIG. 54A-54B shows a nickase based CapSDA workflow combined with lateral flow direct capture. * represents a PTO bond. Bold nucleotides represent a nickase recognition sequence.

### DEFINITIONS

Ambient temperature: As used herein, the term “ambient temperature” is the temperature of surroundings. In general, the term ambient temperature is to be understood as the temperature of any object or environment surrounding an item. Measuring an ambient temperature can be accomplished by using a thermometer or sensor. The ambient temperature of an item is dependent on the temperature of the surrounding of the item. The surroundings can have any temperature, such as a temperature below 95° C., such as below 90° C., such as below 85° C., such as below 80° C., such as below 75° C., such as below 70° C., such as below 65° C., such as below 60° C., such as below 55° C., such as below 50° C., such as below 45° C., such as below 40° C., such as below 35° C., such as below 30° C., such as below 25° C., such as below 24° C., such as below 23° C., such as below 22° C., such as below 21° C., such as below 20° C. Exemplary ambient temperature ranges include 5° C. to 50° C., such as 10° C. to 40° C., such as 15° C. to 35° C., such as 20° C. to 30° C., such as 20° C. to 25° C., such as 20° C. to 22° C.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest is or comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semipermeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Native nickase site: As used herein refers to a nickase site that is naturally occurring in a nucleic acid sequence (e.g., a nickase site is not introduced by manipulation and/or amplification of a nucleic acid sequence)

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only nonnatural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, Damino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample may be a “crude” sample in that it has been subjected to relatively little processing and/or is complex in that it includes components of relatively varied chemical classes.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

### DETAILED DESCRIPTION OF CERTAIN EMBODIMENTS

In some aspects, the present disclosure provides compositions and methods for ambient amplification of a target nucleic acid. In some embodiments, amplification is followed by detection of a target nucleic acid amplicon (i.e., a DNA cassette).

In some aspects, the present disclosure provides systems that utilize nucleic acid sensor technologies that initially (and, in some embodiments, in a target-sequence dependent manner) hybridize to a target nucleic acid of interest, and uses those systems to generate a detectable output.

### Compositions

The present disclosure provides compositions useful in amplification of a target nucleic acid. In some embodiments, the present disclosure provides compositions useful in amplification and detection of a target nucleic acid.

In some embodiments, the present disclosure provides compositions useful in producing a target amplicon (e.g., a DNA cassettes), preparing multiple copies of nucleic acid identical to the target amplicon (e.g., DNA cassette) and detection of at least one copy of nucleic acid identical or complementary to the target amplicon (e.g., DNA cassette(s)). In some embodiments, compositions of the present disclosure may be used to produce a single stranded DNA cassette (ssDNA) or multiple copies of nucleic acids identical or complementary to the ssDNA cassette, for example, at ambient temperature. In some embodiments, such compositions are used to produce single stranded DNA cassette (ssDNA) or multiple copies of nucleic acids identical or complementary to the ssDNA cassette, for example, at isothermal conditions (e.g., without the need for temperature cycling). In some embodiments, compositions provided herein detect a ssDNA cassette, for example, at ambient temperature. In some embodiments, compositions provided herein detect a ssDNA cassette, for example, at isothermal conditions (e.g., without the need for temperature cycling).

In some embodiments, compositions provided herein comprise a target nucleic acid.

In some embodiments, compositions provided herein comprise an oligonucleotide binder (e.g., a primer and/or a probe). In some embodiments, compositions provided herein comprise one or more oligonucleotide binders (e.g., primers and/or probes). In some embodiments, oligonucleotide binders of the present disclosure are designed to bind specifically to a target nucleic acid.

In some embodiments, a composition comprises a ligase.

In some embodiments, a composition comprises a reverse transcriptase.

In some embodiments, a composition comprises a cleavage enzyme. In some embodiments, a composition comprises a restriction enzyme. In some embodiments, a composition comprises a nickase.

In some embodiments, a composition comprises a single-strand binding protein.

In some embodiments, a composition comprises a strand displacing polymerase.

In some embodiments, a composition comprises dNTPs. In some embodiments, a composition comprises one or more modified dNTPs.

In some embodiments, components and compositions for detecting a ssDNA cassette or multiple copies of nucleic acids identical or complementary to the ssDNA cassette are also included in compositions of the present disclosure. In some embodiments, a composition according to the present disclosure comprises a detectably labeled nucleic acid probe, a guide nucleic acid and a Cas enzyme (e.g., a Cas enzyme having collateral cleavage activity).

### Samples

In some embodiments, a sample comprises a target nucleic acid. In some embodiments a sample is an environmental sample. In some embodiments a sample is a biological sample. In some embodiments, a sample is from a subject. In some embodiments, a sample is from a human subject. In some embodiments, a sample is blood, saliva, sputum, mucus, urine, or stool. In some embodiments, a sample is a swab of a surface in or on a human body. In some embodiments, a sample is a swab of a mucosal surface or membrane. In some embodiments, a sample is nasal swab, a cheek swab, an endocervical swab, a vulvovaginal swab, or a throat swab.

In some embodiments, a sample is processed. In some embodiments, a sample is processed to isolate components of the sample. In some embodiments, a sample is processed to isolate nucleic acids (e.g., RNA and/or DNA). In some embodiments, a sample is processed to isolate RNA. In some embodiments, a sample is processed to isolate DNA. In some embodiments, a sample is processed to separate a double stranded nucleic acids into single stranded nucleic acids.

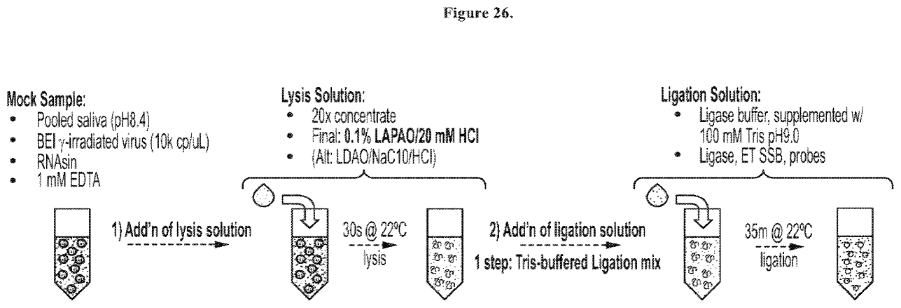

In some embodiments, a sample is prepared or processed to provide a nucleic acid preparation. In some embodiments, a sample is prepared or processed using lysis buffers. In some embodiments, a sample is prepared as in FIG. 26. In some embodiments, a lysis buffer comprises a detergent. In some embodiments, samples (e.g., viral particles and/or cells) are lysed, (e.g., processed) using a zwitterionic detergent (e.g., as in Example 1). In some embodiments, samples (e.g., viral particles and/or cells) are lysed, (e.g., processed) using a zwitterionic detergent as described in 63/358,044 filed on Jul. 1, 2022 herewith and incorporated herein by reference. In some embodiments, such a zwitterionic detergent is selected from the group consisting of LAPAO, LDAO, and DDAO. Certain detergents demonstrate surprising effectiveness (e.g., certain zwitterionic detergents, such as LAPAO, LDAO, and DDAO) for use in lysing viral particles and/or releasing nucleic acids from viral particles, so that a nucleic acid preparation is obtained. Advantages of certain embodiments of provided lysis technologies may include, among other things, that a useful nucleic acid preparation is provided without use of one or more traditional processing steps—such as purification, isolation or extraction steps that are commonly required or utilized to remove detergents.

In some embodiments, the concentration of a zwitterionic detergent is within the range of 0.01% and 10%. In some embodiments, a lysis buffer comprises a zwitterionic detergent and HCl. In some embodiments, the concentration of HCl is within the range of 4 mM and 4M. In some embodiments, the pH of the lysis buffer is within the range of 0 and 6. In some embodiments, a zwitterionic detergent is selected from the group consisting of LAPAO, LDAO, and DDAO. In some embodiments, the concentration of LAPAO is within the range of 0.01% and 10%. In some embodiments, the concentration of LDAO is within the range of 0.02% and 4%. In some embodiments, the lysis buffer further comprises sodium decanoate.

FIG. 27 shows exemplary lysis and nucleic acid processing steps under ambient temperatures. As depicted, a sample containing pooled human saliva plus an inactivated intact virus in a matrix of cell lysate was first treated with a lysis buffer (e.g., comprising a zwitterionic detergent) and then, without traditional processing or “clean-up” steps, it was treated with a “ligation solution”. Resulting in nucleic acid preparations amenable to sensitive detection technologies.

In some embodiments, samples (e.g., viral particles and/or cells) are lysed (e.g., processed) using sodium hydroxide (NaOH). In some embodiments, samples are lysed with NaOH at ambient temperature (e.g., room temperature). In some embodiments, the concentration of NaOH is about 1 mM NaOH to about 200 mM NaOH. In some embodiments, the concentration of NaOH is about 10 mM NaOH to about 100 mM. In some embodiments, samples are lysed with NaOH for about 1 second to about 10 min, such as about 10 seconds to about 8 min, such as about 1 min to about 5 min, such as about 2 min to about 4 min. In some embodiments, samples are treated with NaOH to inhibit or reduce RNase activity. In some embodiments, NaOH releases viral nucleic acids from a viral sample. In some embodiments, NaOH denatures double stranded DNA or RNA (e.g., separates strands).

In some embodiments, samples (e.g., viral particles and/or cells) are lysed (e.g., processed) using potassium hydroxide (KOH). In some embodiments, samples comprising DNA (e.g., dsDNA) are treated with KOH. In some embodiments, samples are lysed with KOH at ambient temperature (e.g., room temperature). In some embodiments, the concentration of KOH is about 1 mM KOH to about 200 mM KOH. In some embodiments, the concentration of KOH is about 10 mM KOH to about 100 mM. In some embodiments, samples are lysed with KOH for about 1 second to about 10 min, such as about 10 seconds to about 8 min, such as about 1 min to about 5 min, such as about 2 min to about 4 min. In some embodiments, samples are treated with KOH to inhibit or reduce RNase activity. In some embodiments, KOH releases viral nucleic acids from a viral sample. In some embodiments, KOH denatures double stranded DNA or RNA (e.g., separates strands). In some such embodiments, KOH denaturation separates dsDNA and produces ssDNA.

In some embodiments, a sample may be a “crude” sample in that it has been subjected to relatively little processing and/or is complex in that it includes components of relatively varied chemical classes.

In some embodiments, a cell free extract is a crude extract. In some embodiments, a cell free extract is generated by a cell-free protein expression system (such as, but not limited to PURExpress).

### Target Nucleic Acids

In some embodiments, technologies (e.g., methods or compositions) provided herein amplify and/or detect one or more target nucleic acid(s). In some embodiments, a target nucleic acid is a deoxyribonucleic acid (DNA). In some embodiments, a target nucleic acid is a ribonucleic acid (RNA). In some embodiments, a target nucleic acid is single stranded. In some embodiments, a target nucleic acid is double stranded.

A person skilled in the art is aware of methods to generate ssDNA from RNA (e.g., reverse transcriptase) or sdDNA (heat denaturation or KOH denaturation). In some embodiments, target RNA is converted to ssDNA. In some embodiments target dsDNA is converted to ssDNA.

In some embodiments, a target nucleic acid is present in a sample. In some embodiments, a sample comprises one or more target nucleic acid(s). In some embodiments, a sample comprises one or more target nucleic acid(s) and nucleic acids other than the one or more target nucleic acid(s). In some embodiments, a target nucleic acid is from a eukaryote. In some embodiments, a target nucleic acid is from a prokaryote. In some embodiments, a target nucleic acid is parasitic (e.g., protozoan), bacterial, viral, or fungal. In some embodiments, a target nucleic acid is human.

In some embodiments, a target nucleic acid comprises a target nucleic acid region. In some embodiments, a target nucleic acid region is a nucleotide sequence to be amplified and/or detected by methods and compositions provided herein.

In some embodiments, a target nucleic acid comprises one or more restriction enzyme recognition sequences. In some embodiments, a target nucleic acid comprises two or more restriction enzyme recognition sequences. In some embodiments, one or more restriction enzyme recognition sequences are native restriction enzyme recognition sequences, i.e., such restriction enzyme recognition sequences are naturally occurring in a target nucleic acid and not added by any manipulation or amplification of a target nucleic acid. In some embodiments, a target nucleic acid comprises a restriction enzyme recognition sequence upstream or 5′ of a target nucleic acid region and a restriction enzyme recognition sequence downstream or 3′ of a target nucleic acid region.

In some embodiments, a restriction enzyme recognition sequence is a nickase recognition sequence. In some embodiments, a target nucleic acid comprises a nickase recognition sequence (e.g., a native nickase recognition sequence). In some embodiments, a target nucleic acid comprises one or more nickase recognition sequences (e.g., a native nickase recognition sequences). In some embodiments, a target nucleic acid comprises one or more nickase recognition sequences (e.g., a native nickase recognition sequences). In some embodiments, a target nucleic acid comprises a nickase recognition sequence upstream or 5′ of a target nucleic acid region and a nickase recognition sequence downstream or 3′ of a target nucleic acid region.

In some embodiments, a target nucleic acid does not comprise a restriction enzyme recognition sequence (e.g., a nickase recognition sequence) or a portion thereof. In some embodiments, a complete restriction enzyme recognition sequence (e.g., nickase recognition sequence) or partial restriction enzyme recognition sequence (e.g., nickase recognition sequence), or complements thereof, is added to a target nucleic acid sequence as provided herein below (e.g., by primers and/or probes).

In some embodiments, a target nucleic acid comprises one or more sequences that is capable of hybridizing to one or more oligonucleotide binders. In some embodiments, a target nucleic acid comprises at least one oligonucleotide binding sequence (i.e., a sequence capable of hybridizing to an oligonucleotide binder). In some embodiments, a target nucleic acid comprises two oligonucleotide binding sequences. In some embodiments, a target nucleic acid comprises a first oligonucleotide binding sequence and a second oligonucleotide binding sequence. For example, the target nucleic acid may comprise a first oligonucleotide binding sequence and a reverse complement of a second oligonucleotide binding sequence, wherein the first oligonucleotide binding sequence and the reverse complement of the second oligonucleotide binding sequences flank a target nucleic acid region. A target nucleic acid region refers to a sequence within the target nucleic acid that is specifically amplified. In some embodiments, an oligonucleotide binding sequence is a primer binding sequence. In some embodiments, an oligonucleotide binding sequence is a probe binding sequence.

In some embodiments, an oligonucleotide binding sequence comprises about 10 to about 16 nucleotides. In some embodiments, nucleotides in an oligonucleotide binding sequence are consecutive nucleotides in the primary sequence of the target nucleic acid (i.e., no additional intervening nucleotides or other molecules between the consecutive nucleotides). In some embodiments, an oligonucleotide binding sequence comprises at least 10, at least 11, at least 12, at least 13, at least 14, at least 15, at least 16 nucleotides. In some embodiments, an oligonucleotide binding sequence comprises at most 16, at most 15, at most 14, at most 13, at most 12, at most 11, at most 10 nucleotides.

### Oligonucleotide Binders

In some embodiments, compositions and methods of the present disclosure comprise one or more oligonucleotide binders (e.g., probes or primers). In some embodiments, compositions and methods of the present disclosure comprise one or more primers (e.g., forward primers, reverse primers, strand displacement amplification (SDA) primers, or combinations thereof). In some embodiments, compositions and methods of present disclosure comprise one or more probes (e.g., a first probe, a second probe, or combinations thereof). In some embodiments, compositions and methods of present disclosure comprise one or more probes and one or more primers.

In some embodiments, compositions and methods provided herein utilize one or more oligonucleotide binders to produce a target amplicon, such as an ssDNA cassette.

In some embodiments, compositions and methods provided herein utilize one or more oligonucleotide binders to amplify a target nucleic acid sequence and/or an ssDNA cassette.

In some embodiments, compositions and methods provided herein utilize one or more oligonucleotide binders to detect a target nucleic acid, or copies thereof.

In some embodiments, an oligonucleotide binder comprises a sequence that is complementary to a target nucleic acid. In some embodiments, an oligonucleotide binder comprises a sequence that is complementary to a SDA primer or a portion thereof (e.g., a SDA primer binding sequence). In some embodiments, an oligonucleotide binder comprises a SDA primer binding sequence. In some embodiments, an oligonucleotide binder comprises a stabilization sequence. In some embodiments, an oligonucleotide binder comprises a blocking molecule. In some embodiments, oligonucleotide binders comprise a nickase recognition site or a partial nickase recognition site, or complements thereof. In some embodiments, oligonucleotide binders (e.g., probes) comprise one or more parts that encodes reporting element components.

### Target Complementary Sequences

In some embodiments, an oligonucleotide binder (e.g., a primer or a probe) comprises a sequence that is complementary to a target nucleic acid sequence.

In some embodiments, an oligonucleotide binder comprises a sequence complementary to a target nucleic acid sequence that comprises a native restriction enzyme recognition sequence or portion thereof. In some embodiments, an oligonucleotide binder comprises a sequence complementary to a native restriction enzyme recognition sequence (e.g., a first native restriction enzyme recognition sequence and/or second native restriction enzyme recognition sequence) or portion thereof.

In some embodiments, a portion of an oligonucleotide binder sequence that is complementary to a target nucleic acid sequence is at the 3′ end of the oligonucleotide binder.

In some embodiments, a sequence (e.g., an oligonucleotide binder sequence) that is complementary to a target nucleic acid sequence is at least 85%, 90%, 91%, 92%, 93%, 94%, 95 96%, 97%, 98%, 99% complementary to a target nucleic acid sequence. In some embodiments, a sequence that is complementary to a target nucleic acid sequence is 100% complementary to the target nucleic acid.

In some embodiments, a sequence that is complementary to a target nucleic acid sequence (e.g., a portion of an oligonucleotide binder sequence that is complementary to a target nucleic acid sequence) comprises about 5 to about 16 nucleotides. In some embodiments, a sequence that is complementary to a target nucleic acid sequence comprises at least 5 nucleotides, at least 6, at least 7, at least 8, at least 9, at least 10, at least 11, at least 12, at least 13, at least 14, at least 15, at least 16 nucleotides. In some embodiments, a nucleic acid sequence that is complementary to a target nucleic acid comprises at the most 16 nucleotides, at the most 15, at the most 14, at the most 13, at the most 12, at the most 11, at the most 10, at the most 9, at the most 8, at the most 7, at the most 6, at the most 5 nucleotides.

### Restriction Enzyme Recognition Sequences

In some embodiments, oligonucleotide binders comprise a complete restriction enzyme recognition sequence, or a sequence complementary thereto. In some embodiments, oligonucleotide binders comprise a partial restriction enzyme recognition sequence, or a sequence complementary thereto.

In some embodiments, oligonucleotide binders comprise a complete nickase recognition sequence, or a sequence complementary thereto. In some embodiments, oligonucleotide binders comprise a partial nickase recognition sequence, or a sequence complementary thereto.

In some embodiments, a nickase recognition sequence is or comprises a nucleotide sequence listed in Table 1 or a sequence that is complement thereto. In some embodiments, a partial nickase recognition sequence comprises a portion of a nickase recognition sequence listed in Table 1 or a sequence that is complement thereto. A nickase recognition sequence and a nickase recognition site are used interchangeably herein.

In some embodiments, a nickase recognition sequence is about 3 to about 7 nucleotides. In some embodiments, a nickase recognition sequence is 3 nucleotides. In some embodiments, a nickase recognition sequence is 4 nucleotides. In some embodiments, a nickase recognition sequence is 5 nucleotides. In some embodiments, a nickase recognition sequence is 6 nucleotides. In some embodiments, a nickase recognition sequence is 7 nucleotides.

In some embodiments, a nickase recognition sequence comprises two cytosine nucleotides followed by a thymine, guanine or adenine nucleotide.

### SDA Primer Binding Sequences

In some embodiments, an oligonucleotide binder (e.g., primer or probe) comprises a SDA primer binding sequence. In some embodiments, a SDA primer binding sequence is located at the 5′ end of an oligonucleotide binder. In some embodiments, a SDA primer binding sequence comprises or consists of about 8 to about 12 nucleotides. In some embodiments, a SDA primer binding sequence comprises at least 8 nucleotides, at least 9, at least 10, at least 11, at least 12 nucleotides. In some embodiments, a SDA primer binding sequence comprises at the most 12 nucleotides, at the most 11, at the most 10, at the most 9, at the most 8 nucleotides.

In some embodiments, a SDA primer binding sequence comprises a complete restriction enzyme recognition sequence (e.g., nickase recognition sequence) or a complement thereof. In some embodiments, a SDA primer binding sequence comprises a partial restriction enzyme recognition sequence (e.g., a portion of a nickase recognition sequence) or a complement thereof. In some embodiments, a nickase recognition sequence is or comprises a nucleotide sequence listed in Table 1 or a complement thereof. In some embodiments, a partial nickase recognition sequence comprises portion of a nickase recognition sequence listed in Table 1 or a sequence that is complement hereto.

In some embodiments, a SDA primer binding sequence comprises (i) a complete restriction enzyme recognition sequence (e.g., nickase recognition sequence), a partial restriction enzyme recognition sequence (e.g., nickase recognition sequence), or complements thereof and (ii) a further SDA primer binding sequence. In some embodiments, a further SDA primer binding sequence comprises about 2 to about 6 nucleotides. In some embodiments, a further SDA primer binding sequence is complementary to a target nucleic acid sequence (e.g., complementary to a nucleotide sequence within a target nucleic acid that is adjacent to an oligonucleotide binding sequence).

### Stabilization Sequences

In some embodiments, an oligonucleotide binder comprises a stabilization sequence. In some embodiments, a stabilization sequence extends from the 5′ end of an oligonucleotide binder. In some embodiments, a stabilization sequence comprises about 8 to about 20 nucleotides. In some embodiments, a stabilization sequence comprises at least 8 nucleotides, at least 9, at least 10, at least 11, at least 12, at least 13, at least 14, at least 15, at least 16, at least 17, at least 18, at least 19, at least 20 nucleotides. In some embodiments, a stabilization sequence comprises at the most 20 nucleotides, at the most 19, at the most 18, at the most 17, at the most 16, at the most 15, at the most 14, at the most 13, at the most 12, at the most 11, at the most 10, at the most 9, at the most 8 nucleotides.

In some embodiments, a stabilization sequence comprises a partial restriction enzyme recognition sequence (e.g., nickase recognition sequence). In some embodiments, a stabilization sequence comprises a partial restriction enzyme recognition sequence (e.g., nickase recognition sequence) and an additional nucleotide sequence. In some embodiments, such additional sequence is about 2 to about 20 nucleotides long. In some embodiments, nucleotides of an additional sequence can be any nucleotides that do not alone or together with the restriction enzyme recognition sequence (e.g., partial nickase recognition sequence) form a complete restriction enzyme recognition sequence (e.g., nickase recognition sequence). In some embodiments, a stabilization sequence does not comprise a restriction enzyme recognition sequence (e.g., partial nickase recognition sequence) or a complete restriction enzyme recognition sequence (e.g., nickase recognition sequence).

In some embodiments, a stabilization sequence comprises a RNA polymerase binding sequence. In some embodiments, a stabilization sequence is or comprises a T7 RNA polymerase promoter. In some embodiments, a RNA polymerase binding sequence is a eukaryotic RNA polymerase binding sequence. In some embodiments, a RNA polymerase binding sequence is a bacteriophage RNA polymerase binding sequence. In some embodiments, a RNA polymerase binding sequence is an RNA polymerase I, RNA polymerase II, RNA polymerase III, RNA polymerase IV, RNA polymerase V, Nr virion RNA polymerase, or T7 RNA polymerase binding sequence. In some embodiments, a RNA polymerase binding sequence is a T7 RNA polymerase binding sequence. In some embodiments, a RNA polymerase binding sequence is a bacterial RNA polymerase binding sequence. In some embodiments, a T7 RNA polymerase promoter is or comprises a TAATACGACTCACTATAGG (SEQ ID NO: 70).

In some embodiments, a stabilization sequence is a linear single stranded sequence.

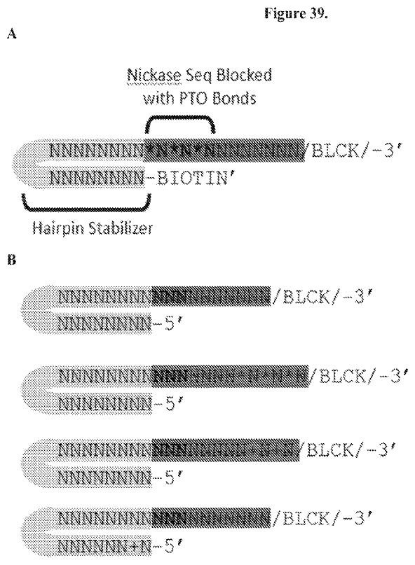

In some embodiments, a stabilization sequence is a hairpin stabilizer. In some embodiments, a hairpin stabilizer comprises a hairpin-loop structure, i.e., a stabilization sequence adapts a folded form where the 5′ end of the stabilization sequence, or a portion thereof, hybridizes to the 3′ end of the hybridization sequence, or a portion thereof, (e.g., a stem stabilizer). In some embodiments, a folded form comprises a stabilizer stem portion and stabilizer loop portion. In some embodiments, a stabilizer loop is single stranded. In some embodiments, a stabilizer stem is double stranded. Exemplary hairpin stabilization sequences are shown in FIG. 39.

### Blocking Molecules

In some embodiments, an oligonucleotide binder (e.g., a primer such as a SDA primer) comprises a 3′ blocking molecule.

In some embodiments, a blocking molecule blocks elongation of a SDA primer (e.g., stops elongation of a nucleotide sequence from proceeding) in the 3′ direction by chemically modifying the 3′ OH group of the 3′ terminal nucleotide of the SDA primer. In some embodiments, the 3′ OH chemical modification blocks the strand displacing polymerase from adding an additional nucleotide to the 3′ terminal nucleotide of the primer.

In some embodiments, a 3′ blocking molecule may also inhibit exponential amplification of primer dimers.

In some embodiments, a 3′ blocking molecule, when bound to the 3′ terminal of a primer, blocks elongation of the SDA primer in the 3′ direction. In some embodiments, a blocking molecule binds to the 3′ OH group of the 3′ terminal nucleotide of the primer.

In some embodiments, a blocking molecule is selected from the group consisting of 3′ddNTP, 3′ Inverted dT, a 3′ carbon chain spacer, a 3′ hexanediol, a 3′ amino spacer, and a 3′ phosphorylation.

In some embodiments, a 3′ddNTP is a dideoxynucleotide triphosphate that does not have a 3′ OH group required for elongation. In some embodiments a deoxynucleotide triphosphate is selected from the group consisting of ddTTP, ddATP, ddGTP, and ddCTP.

In some embodiments, a 3′ Inverted dT has a 3′-3′ linkage that inhibits elongation.

In some embodiments, a 3′ carbon chain spacer is a carbon chain bound to the 3′ OH group blocking elongation. In some embodiments, a carbon chain spacer may be 3C, 4C, 5C, 6C, 7C, 8C, 9C, 10C, 11C, 12C or more in length.

In some embodiments, a 3′ hexanediol is a C6 glycol chain bound to the 3′ OH group blocking elongation.

In some embodiments, a 3′ amino spacer binds to the 3′OH group, of the oligonucleotide binder, required for elongation. In some embodiments, a 3′ amino spacer is a 4C, 5C, 6C, 7C, 8C, 9C, 10C, 11C, 12C or more carbon chain with a methoxy group on C1, and an NH2 group bound to the last carbon of the spacer.

In some embodiments, a 3′ phosphorylation is a phosphate group bound to the 3′ OH required for elongation.

In some embodiments, a blocking molecule is hexanediol (3C6). In some embodiments, a blocking molecule is a 3SpC3.

### Sensor Parts

In some embodiments, an oligonucleotide binder (e.g., a probe) comprises a first and/or second nucleic acid sensor part. In some embodiments a first probe comprises a first sensory part. In some embodiments, a second probe comprises a second sensory part. A probe pair used herein may comprise a nucleic acid sensor set. A nucleic acid sensor set comprises at least a first nucleic acid sensor part and a second nucleic acid sensor part.

In some embodiments, a first nucleic acid sensor part comprises a sequence that is, encodes, or templates at least one reporting element. In some embodiments, a second nucleic acid sensor part comprises a sequence that is, encodes, or templates at least one reporting element. The first and second nucleic acid sensor parts are related to one another in that, when the system is in contact with a sample comprising a target nucleic acid, hybridization of the target nucleic acid with both of the first and second nucleic acid sensor parts juxtaposes the first and second nucleic acid sensor parts with one another so that the juxtaposed parts are susceptible to linkage by one or more of (i) ligation to generate a ligation product and/or (ii) templated coping to generate a linked template product (e.g., they form a “nicked arrangement”).

In some embodiments, one or both nucleic acid sensor parts may comprise a templating element that directs synthesis of a single, intact strand complementary to the nicked arrangement. For example, where a templating element is or comprises a promoter and/or one or more transcriptional regulatory elements, the system may be or comprise an RNA polymerase; where a templating element is or comprises an origin of replication and/or a binding site for an extendible primer, the system may be or comprise a DNA polymerase (which, in some embodiments, may be a thermostable DNA polymerase, particularly if the juxtaposed strand includes a sequence element corresponding to a second extendible primer and the system includes an appropriate pair of primers to amplify a duplex of the juxtaposed strand and its complement).

In some embodiments, linkage of first and second nucleic acid sensor parts generates a nucleic acid strand (i.e., a linked strand) that includes both of the first and second reporting elements (or their complements), which nucleic acid strand is a reporter in that it, or its complement (e.g., generated by transcription or extension [e.g., primed extension]), or an expression product (e.g., generated by transcription and/or translation) of either, is detectable or otherwise generates or participates in generation of a detectable signal indicative of presence and/or amount of the target nucleic acid in the sample.

In some embodiments, a linked strand may be transcribed and/or translated (e.g., via cell-free components such as a cell free protein synthesis expression system (CFPS)).

In some embodiments, linkage as provided herein generates a detectable output. In some embodiments, such detectable output is or is generated by a polypeptide. In some embodiments, a detectable output may be or comprise a catalytic output; in some embodiments, a detectable output may be or comprise a non-catalytic output.

In some embodiments, a catalytic output is or is generated by an enzyme that catalyzes a reaction, e.g., converting one or more substrates to one or more detectable. In some embodiments, a non-catalytic output is or generates a detectable nucleic acid or polypeptide (e.g., that act as an antigen or other specific binding ligand).

Among other things, the present disclosure provides technology formats in which a detectable output is amenable to lateral flow analysis (e.g., is, comprises, or generates a product that is detectable by lateral flow). In some embodiments, the present disclosure provides an insight that coupling linkage mediated detection technologies with lateral flow assessment technologies may particularly facilitate multiplexed analyses (e.g., simultaneous detection of a plurality of products amenable to lateral flow). Furthermore, the present disclosure teaches that such coupling may have particular advantages that permit effective multiplexed analysis of products of different chemical class (e.g., two or more of nucleic acids, metals, polypeptides, small molecules, antibodies or fragments thereof etc.).

In some embodiments, a sensory part is positioned adjacent to the binder hybridization sequence.

In some embodiments, technologies provided herein may include one or more bridging oligonucleotides (e.g., which may be referred to as “gap filling oligonucleotides (“GFO”) that hybridize to the target site between other, e.g., the first and second nucleic acid sensor parts. In some embodiments, a probe set, e.g., a first and second probe, comprises nucleic acid sensors includes only two (i.e., first and second) nucleic acid sensor parts. In some embodiments, technologies provided herein may include one or more bridging oligonucleotides that hybridize to the target site between the first and second nucleic acid sensors.

In some embodiments, a GFO can reduce background or off-target signal. In some embodiments, no detectable output is generated by ligation of a first nucleic acid sensor part and a second nucleic acid sensor part in the absence of a GFO. In some embodiments, an output generated by ligation of a first nucleic acid sensor part and a second nucleic acid sensor part in the absence of a GFO is not a reporting element. In some embodiments, an output generated by ligation of a first nucleic acid sensor part and a GFO or a second nucleic acid sensor part and a GFO is not a reporting element.

In some embodiments, a GFO comprises a primer element. In some embodiments, linkage of a first nucleic acid sensor part, a second nucleic acid sensor part, and a GFO generates a nucleic acid strand (i.e., a linked strand) that includes both of the first and second reporting elements (or their complements), which nucleic acid strand is a reporter in that it, or its complement (e.g., generated by transcription or extension [e.g., primed extension]), or an expression product (e.g., generated by transcription and/or translation) of either, is detectable or otherwise generates or participates in generation of a detectable signal indicative of presence and/or amount of the target nucleic acid in the sample. In some embodiments, a linked strand is amplified (e.g., by a polymerase chain reaction e.g., isothermeral rolling circle amplification). In some embodiments, amplification utilizes at least a primer element in a GFO.

In some embodiments, a GFO comprises one or more nucleic acid sensor parts comprising one or more sequences that is/are, encodes, or templates at least one reporting element. In some embodiments, a GFO comprising one or more sequences that is/are, encodes, or templates at least one reporting element

### Modifications

In some embodiments, an oligonucleotide binder provided herein comprises one or more modified nucleotides (e.g., modified ribonucleotides, modified deoxyribonucleotides, or a combination hereof).

In some embodiments, oligonucleotide binders are modified such that the phosphodiester bond of restriction enzyme recognition sequence on one of the strands is protected using a nuclease resistant modification. In some embodiments, a nuclease resistant modification comprises phosphorothioate (PTO), boranophosphate, methylphosphate or a peptide internucleotide linkage. In some embodiments, modified internucleotide linkages, e.g., PTO linkages, can be chemically synthesized within oligonucleotide probes and primers or integrated into a double stranded nucleic acid by a polymerase, such as by using one or more alpha thiol modified deoxynucleotide. In some embodiments, an oligonucleotide is a modified oligonucleotide, wherein the internucleotide linkages are PTO linkages.

In some embodiments, dNTPs provided herein comprise one or more modified nucleotides.

In some embodiments, a modified nucleotide is selected from the group consisting of an alpha thiol nucleotide, Borano derivatives, 2′-O-Methyl (2′OMe) modified bases and 2′-Fluoro bases. In some embodiments, an oligonucleotide binder comprises one or more alpha nucleotide binders. One of skill will understand a restriction enzyme is able to cleavage both strands in a double stranded DNA. In some embodiments, incorporation of modified nucleotides into one strand of a double stranded DNA prevents cutting of both strands by a restriction enzyme. In some embodiments, incorporation of modified nucleotides into one strand of a double stranded DNA allows a restriction enzyme to only cleave the unmodified strand and leaves the modified strand intact.

In some embodiments, a modified nucleotide is a peptide nucleic acid (PNA). In some embodiments, a modified nucleotide is a locked nucleic acid (LNA). Peptide nucleic acids, locked nucleic acids, or a combination hereof may be used to alter primer Tm and/or specificity. Primers comprising peptide nucleotides, locked nucleotides, or a combination may be particularly useful in methods of detecting a target nucleotide sequence having one or more SNP sites in order to increase specificity.

In some embodiments, a modified nucleotide is a 2′-Fluoro-nucleic acid or a 2′-O-methyl-nucleic acid. Primers comprising 2′-Fluoro-nucleic acid modifications, 2′-O-methyl-nucleic acid modifications or a combination have increased nuclease resistance compared to non-modified primers, as well as increased Tm of the 2′-Fluoro-nucleic acid modifications and/or 2′-O-methyl-nucleotide modified domain(s).

In some embodiments, a modified deoxyribonucleotide is a phosphorothioated deoxyribonucleotide. In some embodiments, a modified deoxyribonucleotide is a phosphodiester deoxyribonucleotide. In some embodiments, a modified deoxyribonucleotide as provided herein destabilizes helices. In some embodiments, a nucleic acid comprising a modified deoxyribonucleotide melts at lower temperatures relative to a control without modified deoxyribonucleotide. In some embodiments, a nucleic acid comprising a modified deoxyribonucleotide can be amplified at lower temperatures relative to a control without modified deoxyribonucleotide.

In some embodiments, a primer comprises a modified nucleotide in place of at least one guanine or adenine. In some embodiments, a modified nucleotide is a 2-Aminopurine (e.g., a purine analog of guanine and adenine). In some embodiments, a primer comprising a 2-Aminopurine is useful in fluorescence readouts.

In some embodiments, one of more of the modifications listed herein provides primers that are more resistant to nucleases and/or proteases compared to primers or other nucleotides without any modifications.

### Exemplary Oligonucleotides

### Reverse Primers

In some embodiments, compositions and methods of the present disclosure comprise one or more reverse primers. In some embodiments, an oligonucleotide binder is a reverse primer. In some embodiments, a reverse primer comprises a nucleic acid sequence complementary to a target nucleic acid (e.g., a RNA target polynucleotide). In some embodiments, a reverse primer comprises a sequence complementary to a target nucleic acid at the 3′ end of the reverse primer. In some embodiments, a reverse primer comprises a SDA primer binding sequence. In some embodiments, a reverse primer comprises a stabilization sequence.

In some embodiments, a reverse primer comprises from the 5′ end to the 3′ end a stabilization sequence, a SDA primer binding sequence and a sequence that is complementary to a target nucleic acid. In some embodiments, a stabilization sequence and a SDA primer binding sequence are separated by one or more nucleotides. In some embodiments, a SDA primer binding sequence and a nucleic acid sequence that is complementary to a target nucleic acid are separated by one or more nucleotides. In some embodiments, a stabilization sequence comprises a partial nickase recognition sequence or complement thereof. In some embodiments, a stabilization sequence and a SDA primer binding sequence together form a complete restriction enzyme recognition sequence (e.g., a nickase recognition sequence). In some embodiments, a stabilization sequence comprises restriction enzyme recognition sequence (e.g., a complete nickase recognition sequence).

In some embodiments, a reverse primer comprises a complete restriction enzyme recognition sequence (e.g., a complete nickase recognition sequence) or complement thereof. In some embodiments, a reverse primer comprises a partial restriction enzyme recognition sequence (e.g., a nickase recognition sequence) or complement thereof.

### Forward Primers

In some embodiments, compositions and methods of the present disclosure comprise one or more forward primers. In some embodiments, an oligonucleotide binder is a forward primer. In some embodiments, a forward primer comprises a nucleic acid sequence complementary a target nucleic acid (e.g., a RNA and/or DNA target polynucleotide). In some embodiments, a sequence complementary to a target nucleic acid is at the 3′ end of the forward primer. In some embodiments, a forward primer comprises a SDA primer binding sequence.

In some embodiments, a forward primer comprises from 5′ end to 3′ end a SDA primer binding sequence and a nucleic acid sequence that is complementary to a target nucleic acid. In some embodiments, a SDA primer binding sequence and a nucleic acid sequence that is complementary to a target nucleic acid are separated by one or more nucleotides. In some embodiments, a SDA primer binding sequence comprises a partial restriction enzyme recognition sequence (e.g., a partial nickase recognition sequence).

In some embodiments, a forward primer comprises a partial restriction enzyme recognition sequence (e.g., a partial nickase recognition sequence).

### Bump Primers

In some embodiments, compositions and methods of the present disclosure comprise one or more bump primers. In some embodiments, a bump primer is complementary to a target nucleic acid sequence and binds upstream of a primer (i.e., at the 5′ end of the target nucleic acid relative to the binding to the primer). In some embodiments, a bump primer is useful when separating a newly synthesized strand of DNA from its template. In some embodiments, a bump primer binds to a DNA template upstream of a forward primer and thereby separates the synthesized strand ssDNA generated by the forward primer.

In some embodiments, one or more bump primers are not used when the sample is lysed using KOH.

### Probes

In some embodiments, compositions and methods of the present disclosure comprise one or more probes. In some embodiments, an oligonucleotide is a probe. In some embodiments, a probe comprises a nucleic acid sequence complementary to a target nucleic acid. In some embodiments, a probe comprises a SDA primer binding sequence. In some embodiment, a probe comprises a first and/or second nucleic acid sensor part, as described herein above. In some embodiments, when the first and second nucleic acid sensor parts are ligated together, they generate an ssDNA cassette that encodes at least one reporter and comprises one or more SDA primer binding sequences.

A probe is a probe as described in PCT Publication, WO 2020/037038, entitled “In vitro detection of nucleic acid” and published 20 Feb. 2020; PCT Publication WO 2020/191376, entitled “System” and published 24 Sep. 2020; PCT publication WO 2021/050560, entitled “System” and published 18 Mar. 2021, the content of each which is incorporated herein by reference in its entirety.

In some embodiments, a probe comprises a restriction enzyme recognition sequence (e.g., nickase recognition sequence). In some embodiments, a probe comprises a partial restriction enzyme recognition sequence (e.g., a partial nickase recognition sequence).

In some embodiments, a capture probe is a biotinylated capture probe. In some embodiments, a capture probe has a 5′ biotin modification. In some embodiments, a capture probe is about 10 to about 20 nucleotides. In some embodiments, a capture probe comprises a 3′ blocking molecule. In some embodiments, a capture probe comprises a stabilization sequence (e.g., a linear single stranded sequence, a hairpin stabilizer or a combination thereof). In some embodiments, a capture probe comprises a restriction enzyme recognition sequence, such as a nickase recognition sequence. In some embodiments, a restriction enzyme recognition sequence and/or nickase recognition sequence comprises one or more modifications (e.g., PTO bonds). In some embodiments, a capture probe comprises a target nucleic acid sequence or complement thereof. In some embodiments, a capture probe comprises a repeat strip pull down sequence. In some embodiments, a strip pull down sequence is a nucleotide sequence of SEQ ID NO: 71 (TGTATGTATGTATGA).

In some embodiments, a probe is a conjugate probe. In some embodiments, a conjugate capture probe is about 10 to about 20 nucleotides. In some embodiments, a conjugate capture probe comprises a target nucleic acid sequence or complement thereof. In some embodiments, a conjugate capture probe comprises a repeat strip pull down sequence. In some embodiments, a strip pull down sequence is a nucleotide sequence of SEQ ID NO: 71.

### Strand Displacement Amplification (SDA) Primers

In some embodiments, compositions and methods provided herein comprise one or more strand displacement amplification (SDA) primers. In some embodiments, using short capped SDA primer(s) during SDA amplification prevents nonspecific dimerization at low temperatures where DNA hybridization is less specific.

In some embodiments, an oligonucleotide binder is a SDA primer. In some embodiments, a SDA primer comprises a nucleotide sequence that is complementary to a target nucleic acid sequence. In some embodiments, a SDA primer comprise a sequence complementary to a native restriction enzyme recognition sequence (e.g., a native nickase recognition sequence). In some embodiments, a SDA primer comprises a nucleotide sequence that is complementary to a SDA primer binding sequence. In some embodiments, a SDA primer comprises a complete restriction enzyme recognition sequence (e.g., nickase recognition sequence) or a complement thereof. In some embodiments, a SDA primer comprises a blocking molecule. In some embodiments, a SDA primer comprises a 3′ blocking molecule. In some embodiments, a blocking molecule blocks elongation of a SDA primer (e.g., stops elongation of a nucleotide sequence from proceeding) in the 3′ direction. In some embodiments, a SDA primer comprises a stabilization sequence.

In some embodiments, a SDA primer hybridizes to a target nucleic acid sequence or complement thereof. In some embodiments, a SDA primer binds to a SDA primer binding sequence introduced to the ssDNA cassette by an oligonucleotide binder (e.g., primer and/or probes) or complement thereof.

In some embodiments, following binding of a SDA primer to a complementary target nucleic acid sequence comprising a native restriction enzyme recognition sequence (e.g., native nickase recognition sequence) or to a SDA primer binding sequence in an ssDNA cassette and subsequent polymerase-based elongation, a double stranded restriction enzyme recognition sequence (e.g., nickase recognition sequence) is produced (e.g., the amplicon generated comprises a double stranded restriction enzyme recognition sequence (e.g., nickase recognition sequence). When nicked and subsequent polymerase-based elongated a reverse complement of the target nucleic acid sequence or ssDNA is produced. Nicking can occur by using a nickase or a restriction enzyme in combination with incorporation of one or more modified dNTP into one of the stranded of the double stranded restriction enzyme recognition sequence, ensuring that only one strand is cleaved (e.g., the polymerase elongation or within the SDA primer).

In some embodiments, compositions and methods of the present disclosure comprise a SDA primer useful in amplifying a target nucleic acid sequence (e.g., comprising a native restriction enzyme recognition sequence, such as a native nickase recognition sequence). In some embodiments, compositions and methods of the present disclosure comprise a SDA primer useful in amplifying an ssDNA cassette. In some embodiments, compositions and methods provided herein produce multiple copies of nucleic acids identical to the ssDNA cassette and/or target nucleic acid sequence having one or more native restriction enzyme recognition sequences (e.g., native nickase recognition sequences). In some embodiments, an ssDNA cassette is amplified using a SDA primer as provided herein generating a plurality of amplified ssDNA cassettes.

In some embodiments, a SDA primer comprises a 3′ blocking molecule. In some embodiments, a SDA primer having a 3′ blocking molecule prevents nonspecific dimerization at low temperatures (e.g., ambient temperatures) where DNA hybridization is less specific.

In some embodiments, a SDA primer is about 16 to about 33 nucleotides. In some embodiments, a SDA primer is at the most 33 nucleotides, such as at the most 32, such as at the most 31, such as at the most 30, such as at the most 29, such as at the most 28, such as at the most 27, such as at the most 26, such as at the most 25, such as at the most 24, such as at the most 23, such as at the most 22, such as at the most 21, such as at the most 20, such as at the most 19, such as at the most 18, such as at the most 17, such as at the most 16 nucleotides.

In some embodiments, a SDA primer comprises a RNA polymerase binding sequence.

In some embodiments, a SDA primer comprises a hairpin stabilizer (e.g., wherein the stabilization sequence form a double stranded hairpin) and a sequence that is complementary to a target nucleic acid sequence comprising a complete nickase recognition sequence.

### Enzymes

### Cleavage Enzymes

In some embodiments, compositions and methods provided herein utilize a cleavage enzyme aiding in amplification of a target nucleic acid sequence. In some embodiments, a cleavage enzyme may cleave one strand of a double stranded target nucleic acid, such as a double-stranded DNA, allowing a polymerase (e.g., a DNA polymerase having strand displacement activity) to extend the target nucleic acid sequence.

In some embodiments, a cleavage enzyme is a restriction enzyme. One of skill in the art is aware restriction enzymes useful for methods and compositions described herein. For example one of skill is aware of enzymes provided by commercial sources for example those listed at www.neb.com/products/restriction-endonucleases.

Restrictions enzymes are proteins isolated from bacteria that cleave DNA sequences at sequence-specific sites, producing DNA fragments with a known sequence at each end. Restriction enzymes are commonly classified into five types, which differ in their structure and whether they cut their DNA substrate at their recognition site, or if the recognition and cleavage sites are separate from one another. Some restriction enzymes cut DNA by making two incisions, once through each sugar-phosphate backbone (i.e. each strand) of the DNA double helix.

In some embodiments, a restriction enzyme is used in combination with one or more modified dNTP. In some embodiments, following hybridization of an oligonucleotide binder to a target nucleic acid sequence, a strand displacement DNA polymerase extends the 3′ end of the oligonucleotide binder using dNTPs and one or more modified dNTP. In some embodiments, a restriction enzyme recognition sequence for a restriction enzyme is formed with one or more modified dNTP base(s) incorporated into the reverse complementary strand acting to block the cleavage of said strand by cleavage of a restriction enzyme. In some embodiments, when a restriction enzyme recognizes its recognition sequence it cleaves only the primer strand that does not include a modified dNTP at the cleavage site thus keeping the other modified strand intact (i.e., a nick). In some embodiments, the nick can be extended by the strand displacement DNA polymerase using the dNTPs and the one or more modified dNTP and displacing the first primer strand.

A number of advantages of using a restriction enzyme exist compared to using e.g., nickases. One example thereof is that a much greater number of restriction enzymes that are not nickases are available than those that are nickases, which means that the restriction enzyme(s) for use in the method or composition of the disclosure can be selected from a large number of potential enzymes to identify those with superior properties for a given application, e.g. reaction temperature, buffer compatibility, stability and reaction rate (sensitivity).

In some embodiments, a cleavage enzyme is a nickase. Nickases (or nicking endonucleases) are a subgroup of restriction enzymes that only cleaves on strand of a dsDNA.

When restriction enzymes bind to their recognition sequences in a DNA sequence, they hydrolyze both strands of a double stranded target nucleic acid (i.e., a duplex) at the same time. Two independent hydrolytic reactions proceed in parallel, most often driven by the presence of two catalytic sites within the restriction enzymes, one for hydrolyzing each strand hereby cleaving the DNA strand. However, nickases are altered restriction enzymes that hydrolyzes only one strand of the duplex, to produce DNA molecules that are “nicked” (e.g., one strand cut) rather than cleaved. Three naturally occurring nickases Nt.BstNBI, Nb.BtsI, and Nb.BsrDI exist. They consist of the large subunits of heterodimeric restriction endonucleases. As such, the catalytic site present in the small subunit that catalyzes cleavage of the other strand is entirely missing. In some embodiments, nickases display no double-strand cleavage activity. In some embodiments, a nickase recognizes a specific nickase recognition sequence within a nucleic acid sequence (e.g., a target polynucleotide).

In some embodiments, compositions and methods of the present disclosure comprise a nickase. In some embodiments, compositions and methods provided herein use nickases to introduce a nick in a double stranded DNA complex comprising a primer (e.g., a capped SDA primer) to allow removal of the 3′ blocking group during elongation of the primer. In some embodiments, compositions and methods provided herein use nickases to introduce a nick in double stranded nucleic acid to allow for strand displacement during elongation. Nick, nicking, and nicked all refer to cleaving one strand of a dsDNA molecule (e.g., a target polynucleotide) by a nickase. The nickases used herein may nick specific nickase recognition sequences. The term, cognate nickase is used to describe the pairing nickase with its corresponding nickase recognition sequence. Cognate pairs are exemplified in Table 1.

In some embodiments, a nickase is cognate to a nickase recognition sequence on an oligonucleotide binder (e.g., a primer or a probe). In some embodiments, a nickase is cognate to a nickase recognition sequence within a target nucleic acid and hence within a target amplicon.

In some embodiments, a nickase binds to a newly formed ssDNA cassette or a target nucleic acid comprising one or more complete nickase recognition site and nicks one strand (e.g., SDA primer), which enable the strand displacing polymerase to remove the 3′ blocking molecule and elongate the SDA primer.

In some embodiments, a nickase binds to a newly formed or already existing double stranded target amplicon and nicks the strand that elongates from the SDA primer. In some embodiments, the nickase nicks the double stranded target amplicon, which contributes to ambient temperature displacement of the strand by the polymerase (e.g., DNA polymerase).

In some embodiments, compositions and methods of present disclosure comprise a nickase. In some embodiments, a nickase is selected from the group consisting of Nt.CviPII, Nb.BbvCI, Nb.Bpu10I, Nb.BsaI, Nb.BsmI, Nb.BsrDI, Nb.BstNBIP, Nb.BstSEIP, Nb.BtsI, Nb.SapI, Nt.AlwI, Nt.BbvCI, Nt.BhaIIIP, Nt.BpulOI, Nt.BpulOIB, Nt.Bsal, Nt.BsmAI, Nt.BsmBI, Nt.BspD6I, Nt.BspQI, Nt.Bst9I, Nt.BstSEI, Nt.CviARORFMP, Nt.CviFRORFAP, Nt.BstNBI, Nt.CviQII, Nt.CviQXI, Nt.EsaSS1198P, Nt.MlyI and Nt.SapI. Nickases are associated to one or more nickase recognition sequences, see Table 1 herein below. A complete nickase recognition sequence is a sequence that would be recognized and nicked by a nickase. A partial nickase recognition site or a part of a nickase recognition site is a sequence that has a portion of a complete recognition sequence.

TABLE 1

| Nickase | Nickase site |
|---|---|
| Nt.CviPII | 5′-CCT-3′, |
| | 5′-CCG-3′, or |
| | 5′-CCA-3′ |
| Nt.BstNBI | 5′-GAGTCNNNNN-3′ |
| Nb.BssSI | 5′-CACGAG-3′ |
| Nb.Bpu10I | 5′-CCTNAGC-3′ |
| Nb.Bsal | 5′-GGTCTC-3′ |
| Nb.BsmI | 5′-GAATGCN-3′ |
| Nb.BsrDI | 5′-GCAATGNN-3′ |
| Nb.BstNBIP | 5′-GAGTC-3′ |
| Nb.BstSEIP | |
| Nb.BtsI | 5′-GCAGTGNN-3′ |
| Nb.SapI | 5′-GCTCTTC-3′ |
| Nt.AlwI | 5′-GGATCNNNNN-3′ |
| Nt.BbvCI | 5′-CCTCAGC-3′ |
| Nt.BhaIIIP | 5′-GAGTC-3′ |
| Nt.BpulOI | 5′-CCTNAGC-3′ |
| Nt.BpulOIB | 5′-CCTNAGC-3′ |
| Nt.Bsal | 5′-GGTCTC-3′ |
| Nt.BsmAI | 5′ -GTCTCNN-3′ |
| Nt.BsmBI | 5′-CGTCTC-3′ |
| Nt.BspD6I | 5′-GAGTC-3′ |
| Nt.BspQI | 5′-GCTCTTC-3′ |
| Nt.Bst9I | 5′-GAGTC-3′ |
| Nt.BstSEI | 5′-GAGTC-3′ |
| Nt.CviARORFMP | 5′-CCD-3′ |
| Nt.CviFRORFAP | |
| Nt.BstNBI | 5′-GAGTC-3′ |
| Nt.CviQII | |
| Nt.CviQXI | 5′-RAG-3′ |
| Nt.EsaSS1198P | 5′-GASTC-3′ |
| Nt.MlyI | 5′-GAGTC-3′ |
| Nt.SapI | 5′-GCTCTTC-3′ |

Those of ordinary skill in the art are aware that various nickases other than those listed in the present disclosure that may be used in the present compositions and/or methods. In some embodiments, a nickase is an Nt.CviPII. In some embodiments, a nickase is an Nb.BbvCI.

In some embodiments, oligonucleotide binders and/or target nucleic acid sequences according to the present disclosure comprises a complete or partial nickase recognition site. In some embodiments, an oligonucleotide binder comprises a partial nickase recognition sequence. In some embodiments, a partial nickase recognition sequence from a SDA primer and a partial nickase recognition sequence from a primer binding sequence form a complete nickase recognition sequence.

Nickases as provided herein may be, for example, active at ambient temperature. In some embodiments, a nickase is stable and or active at temperatures ranging from about 14 to about 45° C., such as about 15 to about 35° C.

### Polymerases

Compositions and methods provided herein utilize, in some embodiments, polymerases having strand displacement activity to amplify a target nucleic acid sequence and/or a ssDNA cassette. In some embodiments, compositions and methods of present disclosure comprise a polymerase. In some embodiments, a polymerase is a polymerase comprising strand displacement activity. A polymerase comprising strand displacement activity is able to displace downstream DNA during elongation. In some embodiments, a polymerase is a DNA polymerase. In some embodiments, a strand displacing polymerase comprises elongation activity at ambient temperature. In some embodiments, a strand displacing polymerase comprises elongation activity at temperatures ranging from about 14 to about 45° C., such as about 15 to about 35° C. In some embodiments, a strand displacing polymerase has elongation activity at room temperature.

In some embodiments, a DNA polymerase having strand displacement activity is selected from the group consisting of Bsu DNA Polymerase I (Bsu DNAP), phi29, Bst 20 DNA Polymerase (Bst DNAP), Klenow Large Fragment (LF), Klenow Exo-, Bsu Large Fragment, Isopol, and Isopol SD+, or variants thereof. In some embodiments, a DNA polymerase having strand displacement activity is a Bsu or a variant thereof. In some embodiments, a DNA polymerase having strand displacement activity is selected from the group consisting of Bsu DNAP, Klenow LF, Klenow Exo-, and Isopol, and Bst DNAP. In some embodiments, a DNA polymerase having strand displacement activity is a Klenow or a variant thereof.

In some embodiments, a DNA polymerase having strand displacement activity is active a low temperature. In some embodiments, a DNA polymerase having strand displacement activity is active at 15° C., at 14° C., at 13° C., at 12° C. In some embodiments, a DNA polymerase having strand displacement activity is active at 14 to about 45° C., such as about 15 to about 35° C.

In some embodiments, a DNA polymerase having strand displacement activity extends a SDA primer from a nick after cutting and displaces the 3′ blocking molecule.

### Single Stranded Binding Proteins

Compositions and methods provided herein, in some embodiments, utilize single strand binding proteins (SSBP). SSBP may stabilize a displaced strand during strand displacing polymerase elongation. In some embodiments, a composition or method provided herein comprises a SSBP. In some embodiments, a SSBP binds to the DNA strand that is displaced by the strand displacing polymerase. In some embodiments, a SSBP binds an oligonucleotide binder (e.g., a primer and/or a probe). In some embodiments, binding of a SSBP to an oligonucleotide binder prevents or reduces non-specific binding. In some embodiments, a SSB protein facilitates polymerase (e.g., DNA polymerase) nick extension. In some embodiments, the SSBP is selected from the group consisting of RpA, T7 gp2.5, T4 Gene 32 Protein (T4gp32), EcoSSB, TaqSSB, and TthSSB. In some embodiments, a SSBP is a T4gp32.

In some embodiment, the concentration of a single stranded binding protein in a composition according to the present disclosure is at least 100 ng/μl, at least 200 ng/μl, at least 300 ng/μl, at least 400 ng/μl.

In some embodiments, T4gp32 is present in the composition within the rage of 100 ng/μl and 500 ng/μl, such as 200 ng/μl to 500 ng/μl, such as 300 ng/μl to 400 ng/μl.

### Reverse Transcriptase

In some embodiments, technologies provided herein comprises a reverse transcriptase. In some embodiments a reverse transcriptase has RNASEh activity. In some embodiments, a reverse transcriptase is selected from the group consisting of MMLV, AMV, Protoscript II, Superscript I and II and II and IV, RTx, GOScript, Sensiscript, Primescript, and Maxima.

### Ligases

In some embodiments, compositions and methods of present disclosure comprise a ligase. In some embodiments, ligase is selected from the group consisting of SplintR, T4 Ligase, T3 Ligase, and T7 Ligase. In some embodiments, a ligase is a SplintR ligase. In some embodiments, a ligase is a T4 DNA ligase. In some embodiments, the concentration of a ligase is within the range of 10 nM to 5 μM (e.g., 500 nM).

### Cas Enzymes

Cas enzymes were originally identified as part of the CRISPR (which stands for “clusters of regularly interspaced short palindromic repeats”)-Cas (which stands for “CRISPR-associated”) systems that provide microbes with adaptive immunity to infectious nucleic acids. Those skilled in the art are aware of an enormous number of Cas enzymes, and of sequence elements and functional characteristics that categorize them into different classes. Class 1 CRISPR-Cas systems have multisubunit effector complexes; Class 2 systems have single-subunit effectors.

At least six different “Types” of Cas proteins have been described; Types I, II, and IV are Class 1 enzymes whereas Types II (including Cas9), V (including Cas12 and Cas14), and VI (including Cas 13) are Class 2 enzymes. Technologies for identifying Cas enzymes, and classifying them (e.g., based on presence, organization, and/or sequence of a RuvC domain and/or one or more other sequence elements) are by now well known in the art. Moreover, many Cas variants have been prepared, and those skilled in the art have a good understanding of structural (e.g., sequence) elements that participate in (e.g., are necessary and/or sufficient for) activities of Cas enzymes.

Those skilled in the art, reading the present application, will appreciate that provided technologies can utilize, in various embodiments, any Cas enzyme (or variant, e.g., engineered variant, thereof) that has cleavage activity which is appropriate to the read-out to be utilized and which is activated by guide nucleic acid binding. Moreover, those skilled in the art, reading the present disclosure, will be well familiar with design choices etc appropriate to match, for example, a particular type of Cas with a particular Cas-activating nucleic acid and/or cleavage substrate (e.g., nucleic acid reporter probe).

Certain Cas enzymes, specifically including Certain Type V and Type VI Cas enzymes, such as Cas12, Cas13, and Cas14 (e.g., Cpf1/Cas12a, C2c2/Cas13a, Cas13b, Cas13c, Cas14a, etc) have been demonstrated to have non-specific nuclease activity that is activated when their guide nucleic acid binds to its target. This non-specific cleavage activity is often referred to as “collateral cleavage”.

Nucleic acid detection systems have recently been developed that utilize the collateral cleavage activity of a Cas protein to detect presence of a target nucleic acid (or, more accurately, a nucleic acid whose nucleotide sequence includes a target site) of interest. In many embodiments, present compositions and methods utilize a Cas enzyme with collateral activity, and detects activation of that activity/cleavage of a nucleic acid reporter probe that is susceptible to Cas enzyme collateral cleavage activity.

In some embodiments, a Cas enzyme is a Cas12 enzyme. In some embodiments, a Cas12 enzyme is an LbaCas12 enzyme. In some embodiments, a Cas enzyme is a Cas13 enzyme. In some embodiments, a Cas13 enzyme is a Cas13a enzyme.

In some embodiments, a Cas enzyme is a thermostable cas enzyme. In some embodiments, a Cas enzyme is thermostable within the range of about 4° C. to about 65° C.

When an appropriate Cas for the type of nucleic acid (i.e., RNA, ssDNA, or dsDNA) present in the Cas activating nucleic acid is contacted with the Cas target nucleic acid, its cleavage (e.g., collateral cleavage) activity is activated, and an appropriate nucleic acid reporter probe is cleaved, resulting in a detectable signal.

### Guide Polynucleotide

In some embodiments, a guide nucleic acid hybridizes to a target nucleic acid region within a target nucleic acid. In some embodiments, a guide nucleic acid is complementary to a target nucleic acid region within a target nucleic acid.

Cas enzymes are activated to cleave (whether specifically or non-specifically) nucleic acids when their guide nucleic acids hybridize with a complementary sequence (a target nucleic acid region or portion thereof). It is well established that guide nucleic acids can be engineered by researchers to hybridize with any target nucleic acid region. Additionally, it is well established that guide nucleic acids may include natural nucleotides, nucleotide analogs, and/or combinations thereof. All of that established knowledge is relevant to, and may be employed in the practice of, the present disclosure.

For example, those skilled in the art will appreciate that a guide nucleic acid may, in some embodiments, have a length (and/or a portion that hybridizes to a Cas recognition element) that is within a range of about 16-28 nucleotides (e.g., about 16, about 17, about 18, about 19, about 20, about 21, about 22, about 23, about 24, about 25, about 26, about 27, or about 28 nucleotides).

One skilled in the art will also appreciate that, in certain embodiments, a guide nucleic acid may have less than 100% perfect complementarity with a relevant Cas recognition element (e.g., may be about 90%, about 91%, about 92%, about 93%, about 94%, about 95%, about 96%, about 97%, about 98%, about 99%, or 100% complementary).

### Buffers

The methods and compositions provided herein comprise buffers that provide reaction conditions suitable for amplification and/or detection of a target nucleic acid. In some embodiments, a buffer comprises components that a skilled person would understand to be in a buffer for DNA amplification. In some embodiments, a composition further comprises a buffer in which ambient amplification of the target nucleic acid can occur. In some embodiments, a buffer comprises deoxynucleotides (dNTPs). In some embodiments, a buffer comprises ribonucleotides (rNTPs). In some embodiments, a buffer comprises Tris or acetate. In some embodiments, a buffer comprises potassium ions (K+). In some embodiments, a buffer comprises potassium acetate or potassium chloride. In some embodiments, a buffer comprises magnesium ions (Mg2+). In some embodiments, a buffer comprises magnesium chloride. In some embodiments, a buffer comprises a polymerase chain reaction enhancer (e.g., Dimethyl sulfoxide (DMSO), Glycerol, Formamide, Bovine Serum Albumin, Ammonium sulfate, polyethylene glycol, gelatin, tween 20, triton X-100, or N,N,N-trimethylglycine (betaine)). In some embodiments, T7 RNA polymerase is active in the buffer. In some embodiments, Cas13 polymerase is active in the buffer. In some embodiments, Cas12 is active in the buffer.

In some embodiments, a buffer is selected from the group consisting of Tris, Phosphate, HEPES, DIPSO, MOBS, HEPPSO, TAPSO, EPPS, Tricine, Gly-Gly, Bicine, HEPBS, TEA, TAPS, AMPD, TABS, AMPSO, CHES, and CAPSO.

In some embodiments, a buffer has a buffering capacity in the rage of about pH 7 to about pH 8.

In some embodiments, compositions and methods of the present disclosure comprise PEG. In some embodiments, a composition or a method comprises 1%-20% PEG, such as 5%-15% PEG. In some embodiments, a composition comprises 10% PEG. In some embodiments, a PEG is a PEG having a molecular weight ranging from 200 to PEG 3350; from 200 to 1000. In some embodiments, a PEG is a PEG 3350.

### Uses and Methods

Those skilled in the art, reading the present disclosure, will appreciate that provided compositions and methods may be utilized in numerous and varying contexts including, but not limited to target nucleic acid synthesis (e.g., producing a ssDNA cassette), amplification, detection, or a combination thereof. In some embodiments, provided compositions and methods may be used to determine or confirm presence or absence of a target nucleic acid. In some embodiments, provided compositions and methods may be used to amplify a target nucleic acid. In some embodiments, provided compositions and methods may be used to detect or quantify amount of target nucleic acid present in a particular sample.

In some embodiments, SDA reactions of the present disclosure proceeds through the amplification at ambient temperatures (e.g., 16-25 C) of an ssDNA input (e.g., ssDNA cassette or an ssDNA target nucleic acid) to a large number of dsDNA amplicons.

In some embodiments, an ssDNA input (e.g., ssDNA cassette or an ssDNA target nucleic acid) is produced using ligation, reverse transcriptase or lysis (e.g., buffers and/or heating) etc.

In some embodiments, the present disclosure provides compositions and methods useful in generating an ssDNA cassette.

In some embodiments, the present disclosure provides compositions and methods useful in amplifying a target nucleic acid.

In some embodiments, the present disclosure provides compositions and methods useful in detecting a target nucleic acid.

### DNA Cassettes

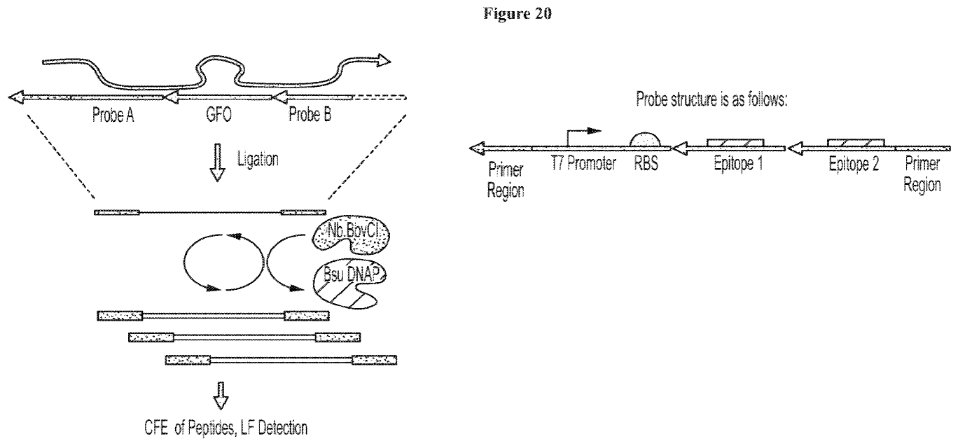

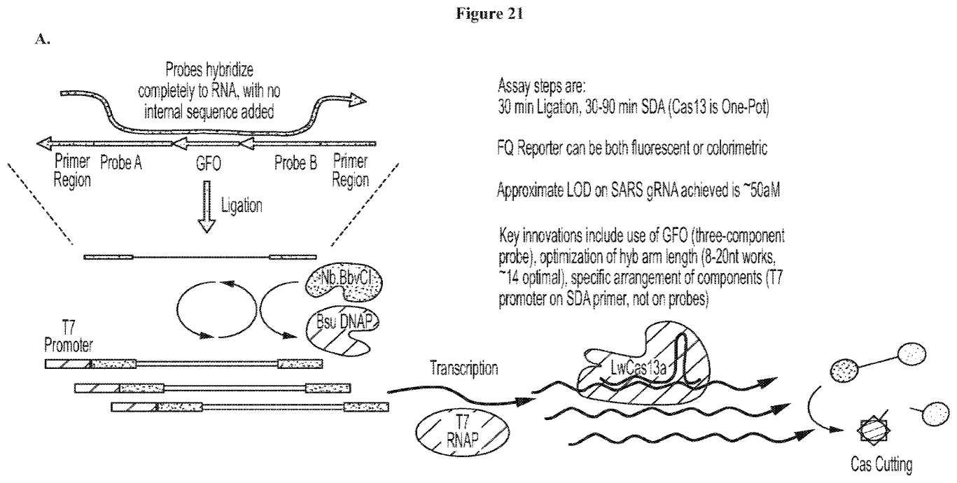

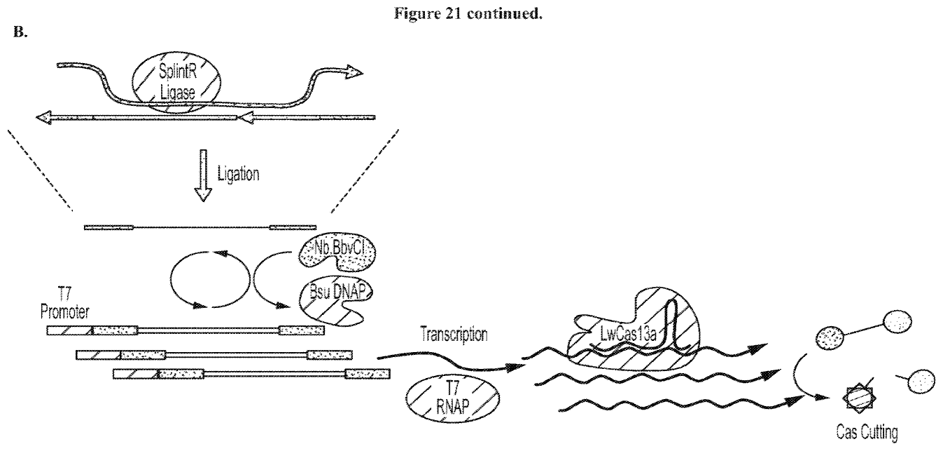

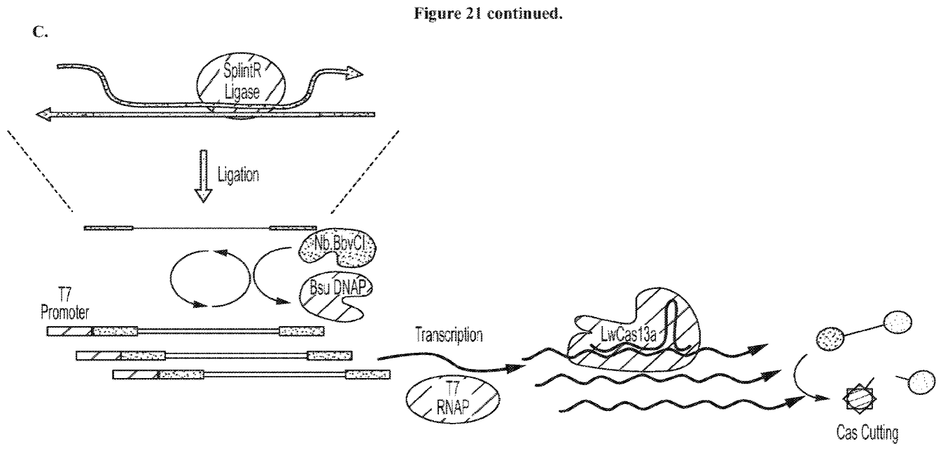

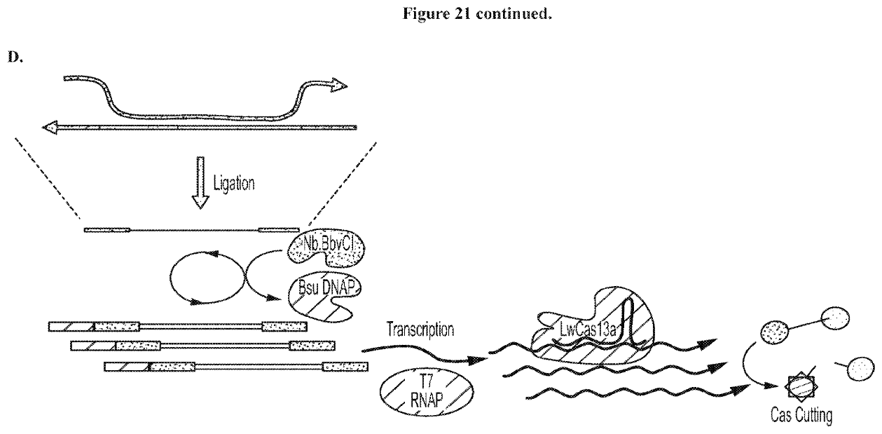

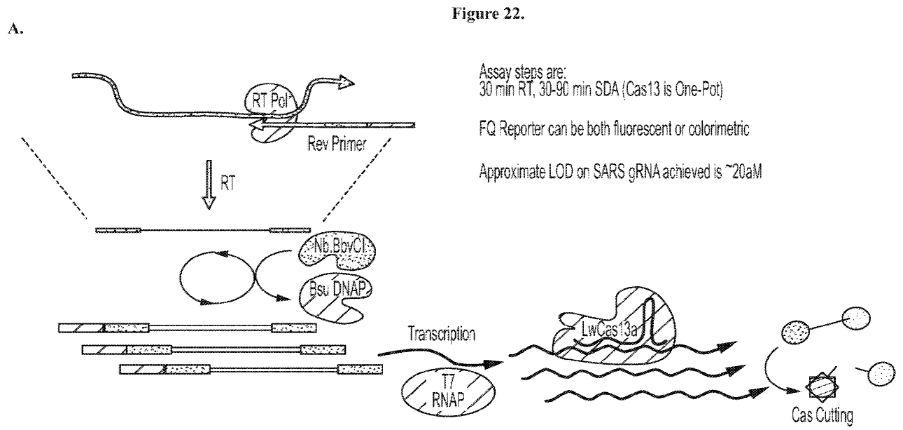

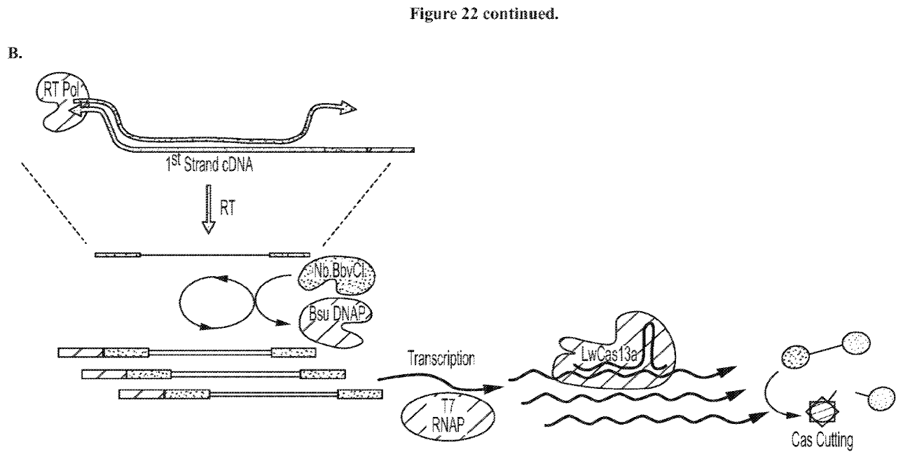

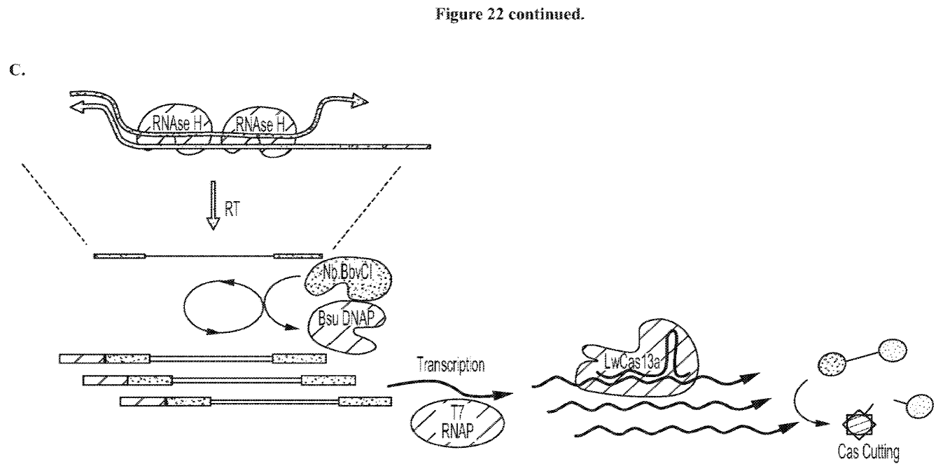

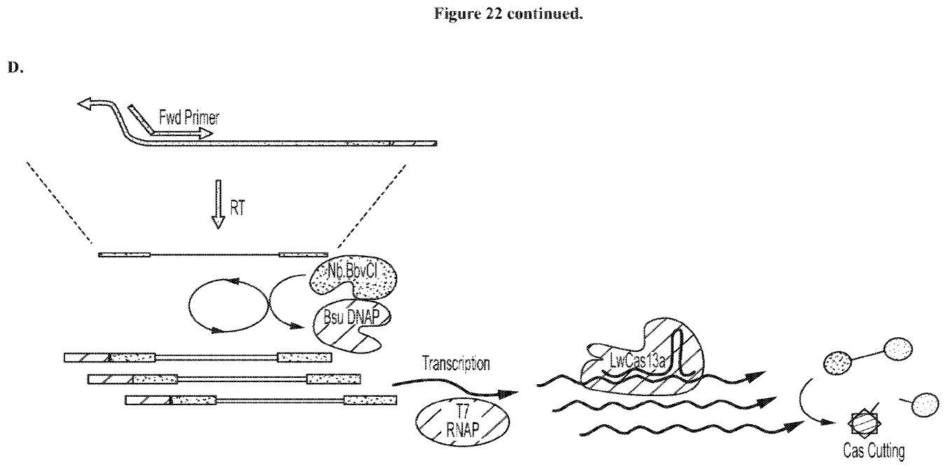

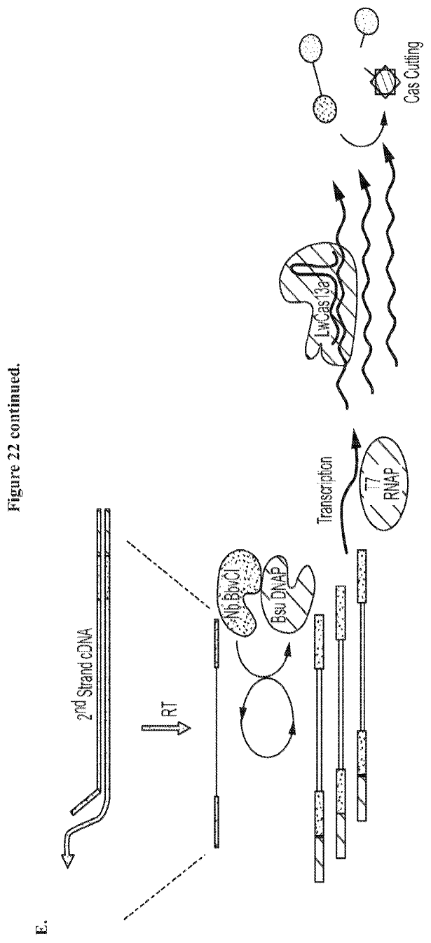

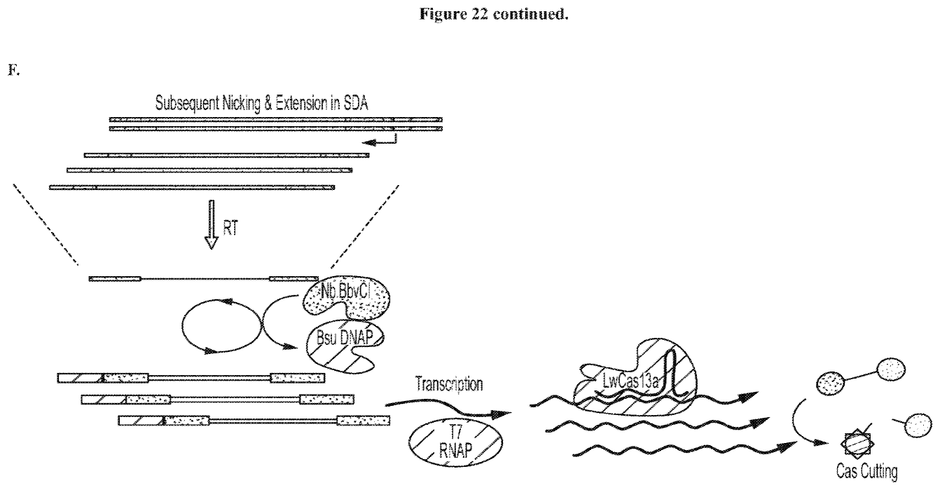

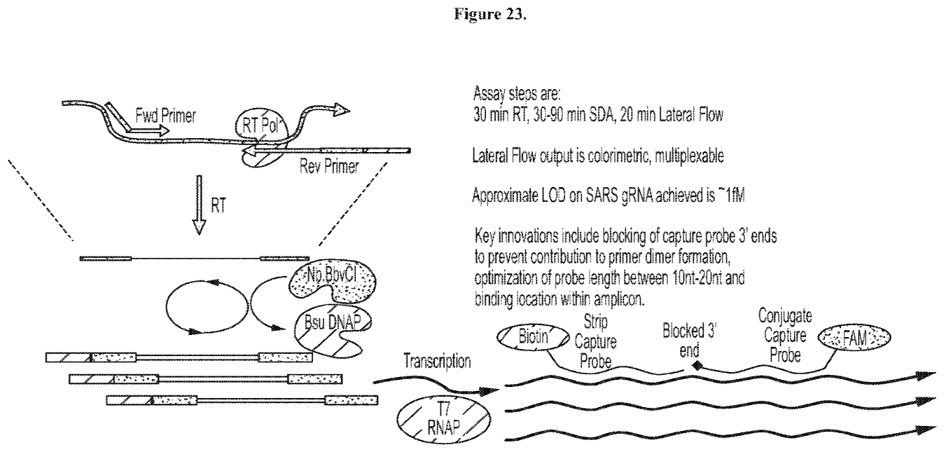

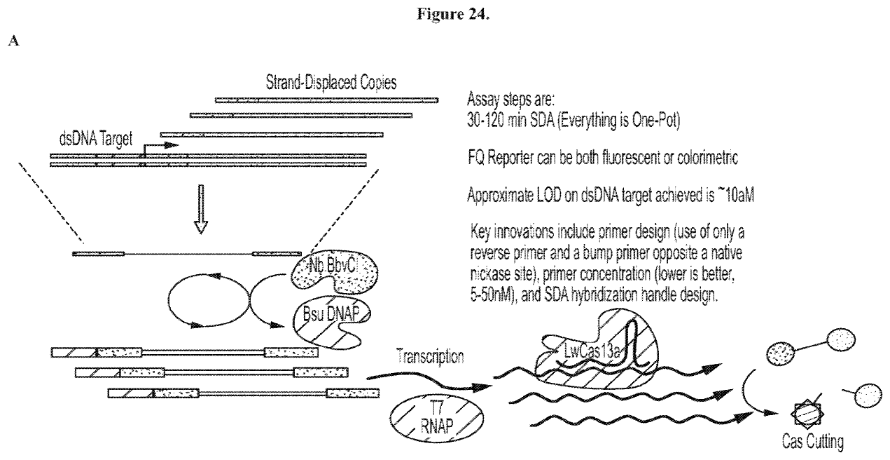

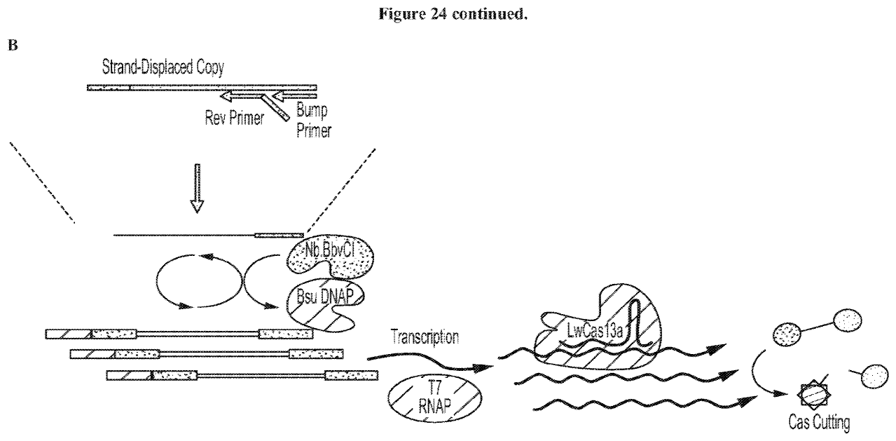

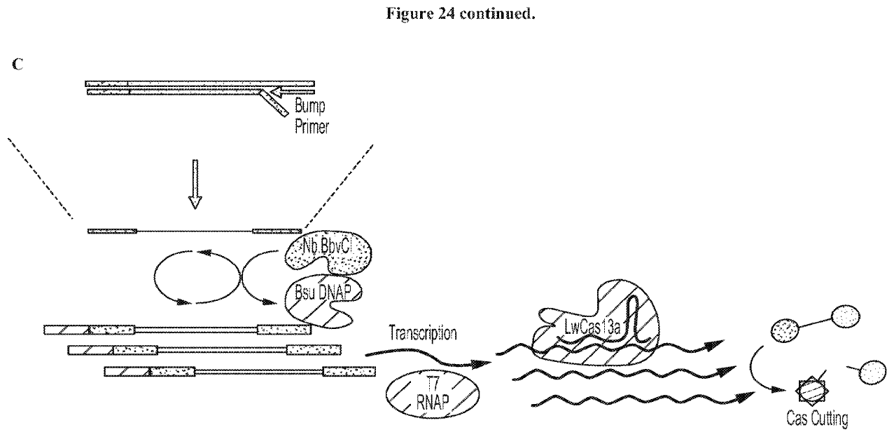

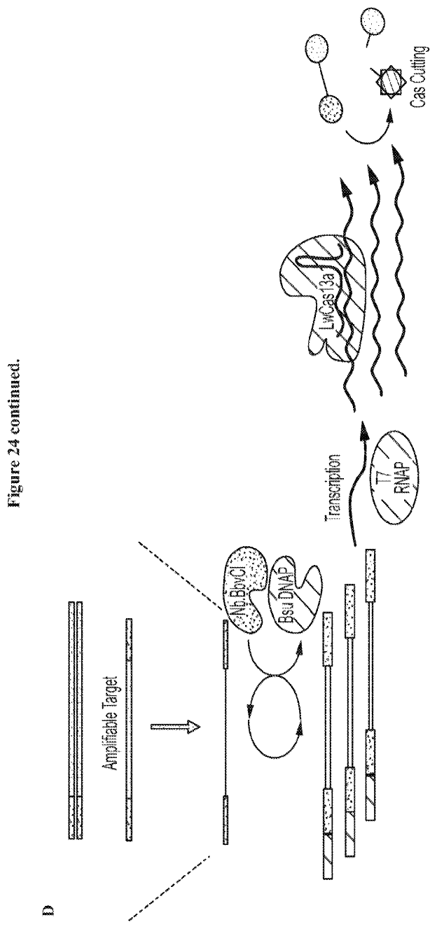

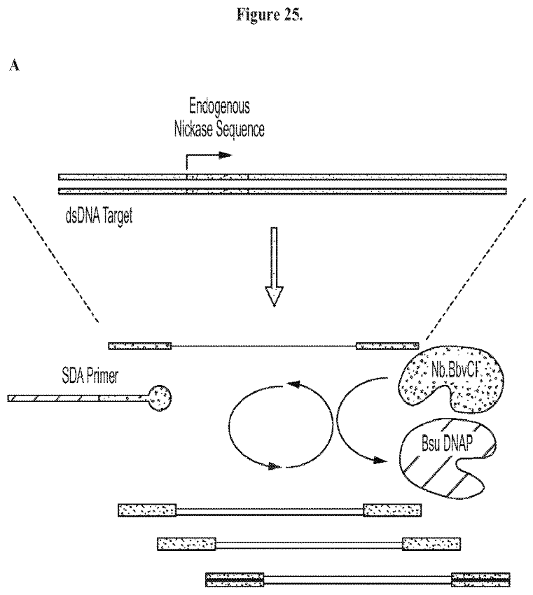

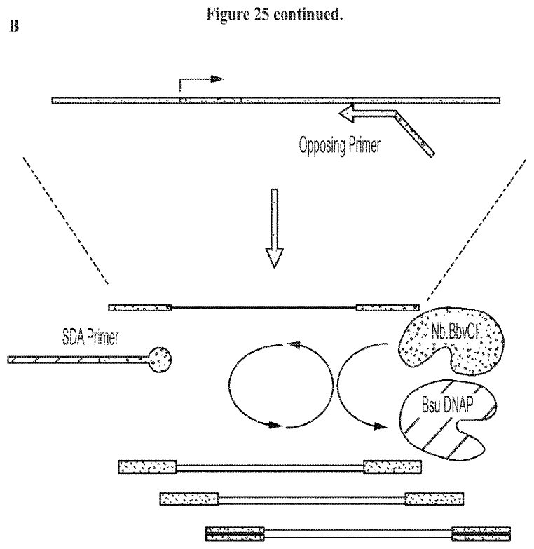

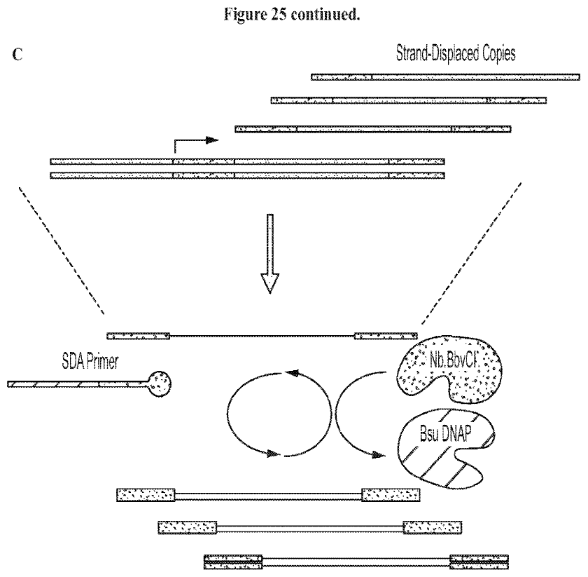

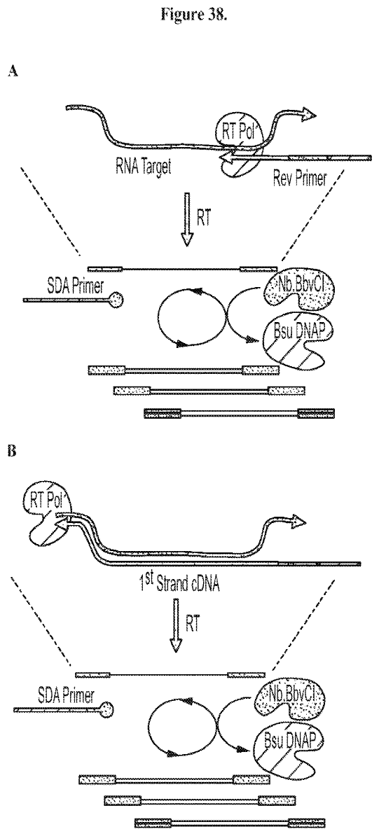

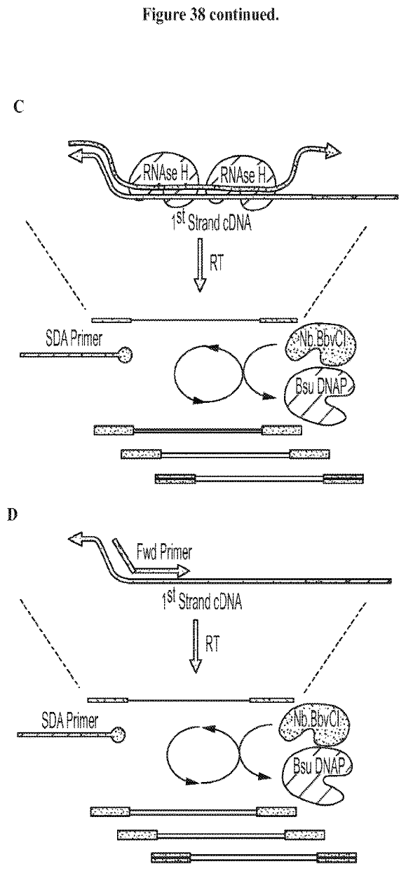

The present disclosure provides various methods to generate an ssDNA cassette, e.g., using a native nickase recognition sequence as shown in FIG. 25, reverse transcription as shown in FIG. 22 and/or FIG. 38, and/or ligation as shown in FIG. 20 and/or FIG. 21.

In some embodiments, an ssDNA cassette is useful in amplification methods as described herein.

In some embodiments, an ssDNA cassette is generated from a target nucleic acid sequence (e.g., a RNA or double stranded DNA) using one or more oligonucleotide binders provided herein. In some embodiments, an ssDNA cassette is generated having SDA primer binding sequences on both ends. In some embodiments, an ssDNA cassette is about 30 to about 300 nucleotides. In some embodiments, a SDA primer hybridizing to an ssDNA cassette is extended by a DNA polymerase (e.g., Bsu DNA polymerase). In some embodiments, a nickase may cut the SDA primer (e.g., Nb.BbvCl) and a DNA polymerase can then extend the nick, thereby discarding the 3′ blocking molecule of the SDA primer.

In some embodiments, an ssDNA cassette is produced by utilizing two native nickase sequences in a target nucleic acid. In some embodiments, a SDA primer as provided herein hybridizes to a target nucleic acid sequence comprising a native nickase recognition sequence followed by nicking of the SDA primer and DNA polymerase extending the nick.

In some embodiments, an ssDNA cassette is produced by utilizing a reverse transcriptase. In some embodiments, a unique ssDNA cassette is generated using oligonucleotide binders as provided herein (e.g., a forward and a reverse primer). In some embodiments, the ssDNA cassette can then be amplified using one or more SDA primers.

In some embodiments, an ssDNA cassette comprises a SDA primer binding sequence as provided herein. In some embodiments, an ssDNA cassette comprises one or more SDA primer binding sequences. In some embodiments, an ssDNA cassette comprises two or more SDA primer binding sequences.

In some embodiments, an ssDNA cassette comprises a target nucleic acid sequence or complement thereof and one or more SDA primer binding sequences. In some embodiments, a SDA primer binding sequence is located at the 3′ end and 5′ end of a target amplicon.

In some embodiments, an ssDNA cassette is produced by utilizing ligation technologies (e.g., utilizing one or more probes as provided herein that hybridizes to a target nucleic acid followed by ligation utilizing gap filling oligos (GFOs)). In some embodiments, the present disclosure utilizes ligation technologies for hybridization with a target nucleic acid. The ligation step is therefore sequence-specific to the target nucleic acid. In some embodiments, a set of ligation oligonucleotides is designed that together hybridize across a target nucleic acid region, adjacent to one another so that activity of a ligase links hybridized oligonucleotides together to form an ssDNA cassette. This ssDNA cassette includes the complement of the entire target site selected and two SDA primer binding sequences. In some embodiments, an ssDNA cassette also includes a Cas recognition element, and typically a templating element, that, prior to ligation, were not part of the same oligonucleotide.

Those skilled in the art will be aware of a variety of systems that utilize sets of ligation oligonucleotides to bring together functional elements located on separate oligonucleotides of the set only when the set becomes linked by ligation, as occurs in the presence of a target nucleic acid of interest, to which each of the oligonucleotides in the set hybridizes at adjacent portions of a target site, is present (see, for example, promoter-ligation-activated-transcription amplification of nucleic acid sequences, as described in U.S. Pat. No. 5,194,370; multiplex ligatable probe amplification [MLPA], as described in U.S. Pat. No. 6,955,901, and Nucleic Acids Research 30:e27, 2002; RNA-splinted nucleic acid ligase activity as described, for example, in Nucleic Acids Research 42:1831, 2013, US patent application US2014/0179539 and Nucleic Acids Research 33:e116, 2016). Those of ordinary skill are therefore well aware of design parameters relevant to construction of oligonucleotides within oligonucleotide sets as provided herein (see also US patent application US2008/0090238).

### Amplification

In some embodiments, the present disclosure provides methods of amplifying a target nucleic acid, such as a double-stranded DNA target nucleic acid comprising a first native restriction enzyme recognition sequence and a second native restriction enzyme recognition sequence on opposite strands separated by a target nucleic acid region, a double-stranded DNA target nucleic acid comprising at least one native restriction enzyme sequence, or a RNA target nucleic acid in a sample.

Amplification may be performed over a wide range of temperatures. The optimal temperature for amplification may be determined by the temperature optimum of the relevant polymerase and restriction enzymes and the melting temperature of the hybridizing regions of the oligonucleotide primers.

In some embodiments, methods provided herein do not use temperature cycling. Furthermore, the amplification step does not require any controlled oscillation of temperature, nor any hot or warm start, pre-heating or a controlled temperature decrease. In some embodiments, methods according to the present disclosure allow for amplification over a wide temperature range e.g., 15° C. to 60°, such as 20° C. to 60° C., such as 15° C. to 45° C. or 15° C. to 35° C.

In some embodiments, amplification is performed at ambient temperature. In some embodiments, amplification is performed without temperature cycling. In some embodiments, amplification is performed under isothermal conditions. In some embodiments, amplification is performed at most 50° C., at most 45° C., at most 40° C., at most 35° C., at most 30° C., at most 25° C., at most 20° C., at most 15° C.

In some embodiments, the present disclosure provides methods of amplifying a double-stranded DNA target nucleic acid comprising a first native restriction enzyme recognition sequence and a second native restriction enzyme recognition sequence on opposite strands separated by a target nucleic acid region in a sample, wherein the method comprises:
(A) contacting the sample with a composition comprising:
   (a) a first SDA primer comprising:
      (i) a sequence complementary to the first native restriction enzyme recognition sequence;
      (ii) a 3′ blocking molecule; and
      (iii) a 5′ stabilization sequence comprising of about 8 to about 20 nucleotides;
   (b) a second SDA primer comprising:
      (i) a sequence complementary to the second native restriction enzyme recognition sequence;
      (ii) a 3′ blocking molecule; and
      (iii) a 5′ stabilization sequence comprising about 8 to about 20 nucleotides;
   (c) a cleavage enzyme;
   (d) a DNA polymerase having strand displacement activity;
   (e) a single stranded binding protein;
   (f) dNTPs; and
   thereby generating a reaction mixture;
(B) incubating the reaction mixture under conditions favorable for
   (i) the first and second SDA primers to hybridize to the double-stranded DNA target nucleic acid in the sample; and
   (ii) amplification of the target nucleic acid,
   thereby generating multiple copies of an amplified target nucleic acid.

In some embodiments, the present disclosure provides methods of amplifying a double-stranded DNA target nucleic acid sequence comprising at least one native restriction enzyme sequence in a sample, wherein the method comprises
(A) contacting the sample, with a composition comprising:
   (a) a forward primer comprising a 3′ nucleic acid sequence complementary to a target nucleic acid sequence downstream of or 5′ to the native nickase recognition sequence and a 5′ SDA primer binding sequence comprising a partial nickase recognition;
   (b) a cleavage enzyme;
   (c) a DNA polymerase having strand displacement activity;
   (d) a single stranded binding protein;
   (e) dNTPs;
   thereby generating a single stranded DNA (ssDNA) cassette;
(B) contacting the ssDNA cassette of step (A) with a composition comprising:
   (f) a first SDA primer comprising:
      (i) a sequence complementary to the at least one native restriction enzyme sequence in the target nucleic acid sequence;
      (ii) a 3′ blocking molecule; and
      (iii) a stabilization sequence comprising about 8 to about 20 nucleotides, and
   (g) a second SDA primer comprising:
      (i) a sequence complementary to the forward primer 5′ SDA primer binding sequence comprising a partial restriction enzyme recognition sequence;
      (ii) a 3′ blocking molecule; and
      (iii) a 5′ stabilization sequence comprising about 8 to about 20 nucleotides, comprising a partial nickase recognition sequence that together with the partial nickase recognition sequence in the 5′ SDA primer binding sequence form a complete restriction enzyme recognition sequence,
   thereby producing a reaction mixture;
(C) incubating the reaction mixture under conditions favorable for generation of multiple copies of nucleic acid identical or complementary to the ssDNA cassette.

In some embodiments, the present disclosure provides methods amplifying a RNA target nucleic acid sequence in a sample comprising:
(A) contacting the sample, with a composition comprising
   (a) a reverse primer comprising:
      (i) a 3′ sequence complementary the RNA target nucleic acid; and
      (ii) a 5′ SDA primer binding sequence, wherein the primer binding sequence comprises a restriction enzyme recognition sequence, a partial restriction enzyme recognition sequence, or complements thereof;
   (b) a reverse transcriptase; and
   (c) a forward primer comprising:
      (i) a 3′ sequence complementary the RNA target nucleic acid; and
      (ii) a 5′ SDA primer binding sequence, wherein the primer binding sequence comprises a restriction enzyme recognition sequence, a partial restriction enzyme recognition sequence, or complements thereof, thereby producing a first reaction mixture;
(B) incubating the first reaction mixture under conditions favorable for generation of a single stranded DNA (ssDNA) cassette;
(C) contacting the ssDNA cassette of (B) with a composition comprising:
   (d) a first SDA primer comprising:
      (i) a sequence complementary to the 5′ SDA primer binding sequence of the reverse primer;
      (ii) a 3′ blocking molecule; and
      (iii) a 5′ stabilization sequence comprising about 8 to about 20 nucleotides, and when the reverse primer comprises only a partial restriction enzyme recognition sequence the first SDA primer comprises a partial restriction enzyme recognition sequence that together with the partial restriction enzyme recognition sequence in the 5′ SDA primer binding sequence of the reverse primer form a complete restriction enzyme recognition sequence;
   (e) a second SDA primer comprising:
      (i) a sequence complementary to the 5′ SDA primer binding sequence of the forward primer;
      (ii) a 3′ blocking molecule; and
      (iii) a 5′ stabilization sequence comprising of about 8 to about 20 nucleotides, when the forward primer comprises only a partial restriction enzyme recognition sequence the first SDA primer comprises a partial restriction enzyme recognition sequence that together with the partial restriction enzyme recognition sequence in the 5′ SDA primer binding sequence of the forward primer form a complete restriction enzyme recognition sequence; and
   (f) at least one cleavage enzyme, polymerase having strand displacement activity, single stranded binding protein, dNTPs; and thereby producing a second reaction mixture;
(D) incubating the second reaction mixture under conditions favorable for generation of multiple copies of a nucleic acid identical or complementary to the ssDNA cassette.

### Detection

Target nucleic acids and/or ssDNA cassettes can be detected by a number of methods. One of skill in the art is aware of various technologies useful in detecting nucleic acids. In some embodiments, the present disclosure provides technologies for detecting target nucleic acids, ssDNA cassettes, or both.

In some embodiments, the present disclosure provides methods of detecting a target nucleic acid sequence, such as a double-stranded DNA target nucleic acid sequence comprising a first native restriction enzyme recognition sequence and a second native restriction enzyme recognition sequence on opposite strands separated by a target nucleic acid region, a double-stranded DNA target nucleic acid sequence comprising at least one native restriction enzyme sequence, or a RNA target nucleic acid sequence in a sample.

In some embodiments, detection technologies comprise, for example, absorbance, CRISPR/Cas detection (e.g., CRISPR-SHERLOCK), FRET, gel electrophoresis, lateral flow, mass spectrometry, PCR, real-time PCR, and/or spectrometry. In some embodiments, detection technologies comprise, for example, chemiluminescence, electrochemical technologies, fluorescence, intercalating dye detection, migration, and/or radiation.

In some embodiments, a step of detecting is performed by detecting a change in florescence as an indication of amplification of the target nucleotide sequence.

In some embodiments, a wherein the change in the fluorescence is an increase in the intensity of fluorescence emission of the detectably labeled nucleic acid probe.

In some embodiments, detection technologies comprise, for example, colorimetric, turbidity, other types of catalysts, molecular beacons and other oligonucleotide-based probes, aptamers, or lateral flow.

In some embodiments, methods according to the present disclosure include non-specific target nucleotide sequence detection. Non-specific nucleotide detection detects nucleotide acid regardless of the particular sequence using a non-specific nucleotide reporter, such as a non-specific fluorescent DNA reporter. Exemplary non-specific nucleic acid reporters include ethidum bromide, propidium iodide, crystal violet, dUTP-conjugated probes, DAIP (4′-,6-diamidino-2-phenylindole), 7-AAD (7-aminoactinomycin D), Hoechst 33258, Hoechst 33342, Hoechst 34580, PICOGREEN, SYBR dyes, such as SYBR Green I, SYBR Green II, SYBR Gold. In some embodiments, method of detecting a target nucleotide sequence utilize a SYBR dye. In some embodiments, methods of detecting a target nucleotide sequence utilize SYBR Green.

In some embodiments, a double stranded DNA binding dye is a minor-groove binding dye. In some embodiments, a mino-groove binding dye is SYBR Green I and II, DAPI, PicoGreen, or a combination. In some embodiments, a double stranded DNA binding dye is an intercalating dye. In some embodiments, an intercalating dye is an Ethidium Bromide, Propidium Iodide, EvaGreen, or a combination.

In some embodiments, provided technologies may be multiplexed. As described herein, in some embodiments, provided technologies may be particularly useful for multiplexed (e.g., simultaneous) analyses of a plurality of products amenable to lateral flow assessment.

In some embodiments, provided technologies may be multiplexed, for example, utilizing different Cas enzymes (and/or readouts) for different target nucleic acid sequences and/or different ligation oligonucleotides.

In some embodiments, detection methods contemplated by the present disclosure include CRISPR based detection methods. Certain CRISPR/Cas enzymes have been identified that exhibit collateral cleavage activity when activated by binding to a target site recognized by the guide polynucleotide with which they are complexed. Cas12, Cas13, and Cas14 are non-limiting examples of CRISPR/Cas enzymes that have been shown to have such collateral cleavage activity. Some CRISPR/Cas enzyme having collateral cleavage activity digests or cleaves single strand nucleic acids (e.g., detectably labeled nucleic acid probes). Collateral activity has been harnessed to develop CRISPR/Cas detection (e.g., diagnostic) technologies that achieve detection of nucleic acids containing a relevant target site (e.g., Cas target nucleic acid), or its complement, in biological and/or environmental sample(s).

In some embodiments, a Cas enzyme has collateral activity.

CRISPR-SHERLOCK is a detection technology comprising steps of: contacting a CRISPR-Cas complex comprising a Cas enzyme with collateral cleavage activity, a guide polynucleotide selected or engineered to be complementary to a target nucleotide sequence (e.g., a Cas target nucleic acid sequence), and a sample potentially comprising a target nucleotide sequence comprising Cas target nucleic acid. In some embodiments, CRISPR/Cas-based detection may be a CRISPR-Cas13-based detection system. In some embodiments, a CRISPR/Cas-based detection system is a CRISPR/Cas12-based detection system. In some embodiments, a CRISPR/Cas13- or CRISPR/Cas12-based detection system is a CRISPR-SHERLOCK detection system. In some embodiments, methods according to the present disclosure utilize a CRISPR-SHERLOCK detection system. In some embodiments, an amplified nucleotide comprising a target nucleotide sequence is incubated with a guide polynucleotide capable of binding the target nucleotide sequence, a detectably labeled nucleic acid probe, and a Cas enzyme.

In some embodiments, a Cas enzyme is a thermostable Cas enzyme. A thermostable Cas enzyme may be a thermostable Cas Enzyme as described in US Publication, US 2023/0002811, entitled “APPLICATION OF CAS PROTEIN, METHOD FOR DETECTING TARGET NUCLEIC ACID MOLECULE AND KIT” and published Jan. 5, 2023; PCT Publication WO 2020/142754, entitled “PROGRAMMABLE NUCLEASE IMPROVEMENTS AND COMPOSITIONS AND METHODS FOR NUCLEIC ACID AMPLIFICATION AND DETECTION” and published Jul. 9, 2021; PCT Publication WO 2021/154866, entitled “IMPROVED DETECTION ASSAYS” and published Aug. 5, 2021; and PCT Publication WO 2023/009526, entitled “IMPROVED CRISPR-CAS TECHNOLOGIES” published Feb. 2, 2023, the content of each which is incorporated herein by reference in its entirety.

In some embodiments, a detectably labeled nucleic acid probe comprises a fluorescent group end and a quenching group. In some embodiments, a detectably labeled nucleic acid probe comprises a fluorescent group at the 5′ end and a quenching group at the 3′ end.

In some embodiments, technologies according to the present disclosure utilizes a guide polynucleotide. A guide polynucleotide is, when incubated with a target polynucleotide, capable of binding to a target nucleic acid region, as an amplified nucleotide comprising a target nucleic acid region.

In some embodiments, a guide polynucleotide is complementary to a target nucleic acid region having one or more SNP mutations.

In some embodiments, generation of increased transcripts can, among other things, decrease the duration of time required for detecting a nucleic acid. In some embodiments, a detection method comprises a CRISPR-Cas based detection method (e.g., CRISPR-SHERLOCK). In some embodiments, a disclosed system for nucleic acid synthesis and/or nucleic acid amplification and/or detection of a nucleic acid occurs in a single reaction vessel (“one-pot” embodiment).

In some embodiments, the present disclosure provides methods of detecting a double-stranded DNA target nucleic acid sequence comprising a first native restriction enzyme recognition sequence and a second native restriction enzyme recognition sequence on opposite strands separated by a target nucleic acid region in a sample, wherein the method comprises:
(A) contacting at least one copy of an amplified target nucleic acid sequence produced by an amplification method as described herein, with a composition comprising:
   (i) a guide nucleic acid comprising a nucleic acid sequence complementary to the DNA cassette; and
   (ii) a Cas enzyme with collateral cleavage activity;
   (iii) a nucleic acid reporter probe susceptible to the collateral cleavage activity, so that the nucleic acid reporter probe has a detectably different first uncleaved state and a second cleaved state; and
(B) detecting cleavage of the nucleic acid reporter probe by detecting a difference between the first state and the second state to determine the presence of the target nucleic acid sequence in the sample.

In some embodiments, the present disclosure provides methods of detecting a double-stranded DNA target nucleic acid sequence comprising a first native restriction enzyme recognition sequence and a second native restriction enzyme recognition sequence on opposite strands separated by a target nucleic acid region in a sample, wherein the method comprises:
(A) contacting at least one copy of an amplified target nucleic acid sequence produced by an amplification method as described herein, with a composition comprising
   (i) a capture probe; and
   (ii) a conjugate capture probe comprising a detectable label and a 3′ blocking entity;
(B) detecting presence of the detectable label bound to a solid substrate or a moiety that permits attachments to a solid material to determine the presence of the target nucleic acid sequence in the sample.

In some embodiments, a capture probe is a biotinylated capture probe. In some embodiments, a capture probe has a 5′ biotin modification. In some embodiments, a capture probe is about 10 to about 20 nucleotides. In some embodiments, a capture probe comprises a 3′ blocking molecule. In some embodiments, a capture probe comprises a stabilization sequence (e.g., a linear single stranded sequence, a hairpin stabilizer or a combination thereof). In some embodiments, a capture probe comprises a restriction enzyme recognition sequence, such as a nickase recognition sequence. In some embodiments, a restriction enzyme recognition sequence and/or nickase recognition sequence comprises one or more modifications (e.g., PTO bonds). In some embodiments, a capture probe comprises a target nucleic acid sequence or complement thereof. In some embodiments, a capture probe comprises a repeat strip pull down sequence. In some embodiments, a strip pull down sequence is a nucleotide sequence of SEQ ID NO: 71 (TGTATGTATGTATGA).

In some embodiments, a conjugate capture probe is about 10 to about 20 nucleotides. In some embodiments, a conjugate capture probe comprises a target nucleic acid sequence or complement thereof. In some embodiments, a conjugate capture probe comprises a repeat strip pull down sequence. In some embodiments, a strip pull down sequence is a nucleotide sequence of SEQ ID NO: 71.

In some embodiments, methods and compositions provided herein can distinguish between target nucleotides that have sequences comprising only a single nucleotide polymorphism(s) (SNPs) to differentiate between said target nucleotides. In some embodiments, provided technologies can be utilized to detect a SNP-containing nucleic acid. In some embodiments, provided technologies can be utilized to detect SNP-containing nucleic acids in a patient-derived sample or samples. In some embodiments, identification of nucleic acids that have sequences comprising a disease-relevant SNP or disease-relevant SNPs can be utilized for diagnosis and/or informing treatment regimens. In some embodiments, use of multiple guide RNAs in accordance with disclosed technologies may further expand or improve on the number of target nucleic acids that can be distinguished from other target nucleic acids.

In some embodiments, disclosed technologies can achieve detection of one or more microbial or other infectious agents in a sample. In some embodiments, such a sample may be or comprise a biological sample, for example which may have been obtained from a subject, and/or an environmental sample, for example which may be or comprise soil, water, etc. In some embodiments, for example, a microbe may be a bacterium, a fungus, a yeast, a protozoa, a parasite, or a virus.

In some embodiments, disclosed technologies can be used in other methods (or in combination) with other technologies that require identification of a particular microbe species or other infectious agent in a sample or, monitoring the presence of microbe or other infectious agent over time (e.g., by identifying the presence of a particular microbial or infectious proteins (antigens), antibodies, antibody genes, detection of certain phenotypes (e.g., bacterial resistance)), monitoring of disease progression and/or outbreak, and antibiotic screening.

In some embodiments, provided technologies achieve certain benefits and/or advantages, e.g., relative to alternative technologies, for example, such as technologies that may utilize conventional amplification methods. For example, in some embodiments, provided technologies provides reduced detection time compared to conventional detection methods.

Alternatively or additionally, in some embodiments, provided technologies may be particularly amenable to use in point-of-care devices. Thus, in some embodiments, provided technologies can guide therapeutic regimens (e.g., selection of treatment type and/or dose and/or duration of treatment).

In some embodiments, water samples such as freshwater samples, wastewater samples, or saline water samples can be evaluated for cleanliness and/or safety, and/or potability, to detect the presence of for example, microbial contamination.

In some embodiments, provided technologies are useful for assessment of environmental samples. For example, household/commercial/industrial surfaces made of any materials including, but not limited to, metal, wood, plastic, rubber, or the like, may be swabbed and tested for contaminants. To give a few examples, soil samples may be tested for the presence of viral particles or fragments thereof, pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, for example to detect the presence of, for example, viral particles, and/or Cryptosporidium parvum, Giardia lamblia, and/or other microbial contamination.

Identification of microbes may be useful and/or needed for any number of applications, and thus any type of sample from any source deemed appropriate by one of skill in the art may be used in accordance with the invention.

In some embodiments, the technologies of the present inventions are useful in genotyping.

The methods provided herein may be performed over a wide range of temperatures. The optimal temperature for each step is determined by the temperature optimum of the relevant polymerase and restriction enzymes and the melting temperature of the hybridizing regions of the oligonucleotide primers.

In some embodiments, methods provided herein do not use temperature cycling. Furthermore, the amplification step does not require any controlled oscillation of temperature, nor any hot or warm start, pre-heating or a controlled temperature decrease. In some embodiments, methods according to the present disclosure allow for amplification over a wide temperature range e.g., 15° C. to 60°, such as 20° C. to 60° C., such as 15° C. to 45° C., or 15° C. to 35° C.

Present methods are effective over a wide range of target nucleic acid concentration, including detection down to very low target nucleic acid copy numbers. In some embodiments, compositions and methods disclosed herein result in robust amplification of the target nucleic acid. Robust amplification of a target nucleic acid refers to compositions and methods that consistently amplify the target nucleic acid to a detectable level. A person of skill will understand that robustness is dependent on the starting concentration of target nucleic acid in the composition or method. For example, compositions and methods provided herein are generally expected to robustly amplify and detect attomolar amounts of a target nucleic acid e.g., at least 2 aM, at least 3 aM, at least 4 aM, at least 5 aM, at least 10 aM, at least 20 aM, at least 50 aM, or at least 100 aM of the target nucleic acid.

In some embodiments, a target nucleic acid is present in a sample and detected at a concentration of at least 2 attomolar (aM). For example, a target nucleic acid may be present in a sample at a concentration of at least 2 aM, at least 3 aM, at least 4 aM, at least 5 aM, at least 10 aM, at least 20 aM, at least 50 aM, or at least 100 aM. In some embodiments, a target nucleic acid is present in a sample at a concentration of 2-5 aM, 2-10 aM, 2-20 aM, 2-40 aM, 2-100 aM, 5-10 aM, 5-20 aM, 5-40 aM, 5-100 aM, 10-20 aM, 10-50 aM, 20-50 aM, 10-100 aM, 50-100 aM, 1-1000 aM, 5-1000 aM, or 50-1000 aM. In some embodiments, a sample comprises other molecules in addition to the target nucleic acid, for example, other non-target nucleic acids.

In some embodiments, methods and compositions of the present disclosure can detect a target nucleic acid present in a sample at a concentration of 1, 2, 3, 4, 5, 6, 7, 8, 9, 10, 11, 12, 13, 14, 15, 16, 17, 18, 19, or 20 copies per microliter. In some embodiments, methods and compositions of the present disclosure can detect a target nucleic acid present a concentration of 1, 2, 3, 4, 5, 6, 7, 8, 9, 10, 11, 12, 13, 14, 15, 16, 17, 18, 19, or 20 copies per reaction.

In some embodiments, a PolyT oligo is added to the detection step. Without wishing to be bound to a particular theory, a PolyT Oligo may soak up excess SSB, such as T4gp32, to avoid that the SSB protein binds to the detection probe (i.e., decrease or remove background noise).

In some embodiments, the present invention provides methods that are suitable for multiplexing. In some embodiments, a number of different capture probes are utilized to capture specific ssDNA cassettes.

### Kit

Provided herein is a kit for performing the present methods. The kit of parts may comprise a composition or a component thereof as described herein.

In some embodiments, a kit is provided for performing methods of amplification, detection, or a combination of a target nucleic acid sequence from a sample. In some embodiments, a kit of parts comprises a composition according to the present disclosure, and/or one or more components thereof. In some embodiments, a kit of parts may comprise a target nucleic acid, an oligonucleotide binder, amplification reagents and/or instructions for use.

In some embodiments, a kit is provided for performing methods of detecting a target nucleic acid sequence in a sample. In some embodiments, a kit of parts comprises a composition according to the present invention. In some such embodiments, a kit of parts may also comprise amplification reagents, device, and/or instructions for use. For example, in some particular embodiments, a kit of parts may comprise a detector system useful for detecting a target nucleotide sequence.

In some embodiments, a kit of parts also comprises a control nucleic acid, such as may be spiked into a sample as described herein.

### Exemplification

### Example 1: Ambient Temperature Viral Particle Lysis

The present Example illustrates effective viral particle lysis achieved at ambient temperature through use of lysis technologies provided herein.

A sample comprising pooled saliva, BEI γ-irradiated SARS-CoV-2 virus (inactivated and intact virus), RNAsin, and EDTA. The starting pH of the pooled saliva was 8.4. The BEI γ-irradiated virus concentration in the sample was 10,000 cp/ul. The RNAsin concentration in the sample was 1 U/μl (N2511, Promega).

Two different stock (20×) lysis buffers were prepared, each of which included a zwitterionic detergent. Specifically, an LAPAO/HCl stock solution, and an LDAO/NaCl10/HCl stock solution were prepared, as set forth in the following Table:

TABLE 2

| Reagents | LAPOA/HCl Stock | LDAO/NaC10/HCl Stock |
|---|---|---|
| LAPAO | 2% (66.5336 mM) | |
| LDAO | | 0.5% (21.796 mM) |
| HCl | 400 mM | 400 mM |
| NaC10 | | 1.2% (65 mM) Stock |

The lysis buffer was added to the mock sample and the combination was incubated at ambient temperature, which in this case was 22° C., for a short period of time (specifically, 30 seconds in the reactions depicted in FIG. 26). The lysis reaction was stopped by adding a neutralization buffer (20× stock solution is 100 mM Tris, pH 9.0). The lysis reaction was performed under different pH conditions, including pH 2.5, pH 4.5, pH 6, pH 6.5, pH 7.5, pH 8 and, pH 10. To achieve the desired pH, the reaction was adjusted with HCl.

Viral particle lysis/nucleic acid preparations were assessed by measuring Ct value of the samples. Poor to no viral particle lysis was indicated by high Ct values, while optimum viral particle lysis was indicated by lower Ct values.

### Positive Control

As a positive control for lysis, an otherwise comparable mock sample comprising pooled saliva, BEI γ-irradiated SARS-CoV-2 virus, RNAsin, and EDTA was exposed to high temperature (specifically, was incubated at 95° C. for 5 minutes), without addition of the lysis buffer (for example LAPAO/HCl stock solution, or an LDAO/NaCl10/HCl solution stock) or other detergents.

### Negative Controls

Three different negative controls were prepared:
(i) “No lysis” comprises the mock sample (pooled saliva, BEI γ-irradiated SARS-CoV-2 virus, RNAsin, and EDTA) without addition of the lysis buffer, including any detergents and HCl. The samples were maintained at ambient temperature. Thus, “No lysis” control samples were neither exposed to detergent(s), HCl nor heat;
(ii) “HCl” comprises the mock sample (pooled saliva, BEI γ-irradiated SARS-CoV-2 virus, RNAsin, and EDTA) without addition of any detergents but it was exposed to HCl. The samples were maintained at ambient temperature. Thus, the “HCl” control samples were neither exposed to detergent(s) nor heat.
(iii) “Neg” or No template control (NTC) comprises only water or a buffer.

As can be seen, for example, with reference to FIG. 27, use of a lysis buffer as provided herein achieved effective lysis comparable to that achieved with the positive control under heating conditions, at least at pH conditions below about 6.0, and particularly below about 4.5. FIG. 27 shows results observed when lysis reactions as were performed under different pH conditions. In particular, FIG. 27 shows that lower Ct values were achieved with lower pH values. Thus, the present Example documents particular effectiveness (i.e., better viral particle lysis) of provided lysis reactions at low pH. Greatest levels of lysis were observed at lowest pH (2.5) tested. However, the limit may not have been reached in these studies.

Among other things, the present Example surprisingly demonstrates that use of a lysis reagent composition such as a zwitterionic detergent (e.g., LAPAO or LDAO) can achieve effective viral particle lysis (e.g., of envelope viral particles) in a biological sample (e.g., a crude sample, and in particular a saliva sample) at low pH (e.g., below 6.5, and preferably below 4.5 including specifically below about pH 3.0), even at ambient (e.g., about 22° C.) temperatures. Moreover, the achieved lysis can be comparable to heat lysis (e.g., that observed with incubated at 95° C. for 5 min).

Moreover, the present Example surprisingly demonstrates that provided effective lysis conditions can be rapidly and gently neutralized by simple addition of Tris, to generate a lysed composition amenable to further nucleic acid processing or manipulation. Among other things, the present disclosure provides an insight that use of certain zwitterionic detergents (e.g., LAPAO and LDAO) as provided herein can achieve a “Goldilocks” effect of sufficient lysis without undesirable inhibition of downstream processing or reaction steps.

Thus, in many embodiments, subsequent manipulation and/or analysis of nucleic acids prepared by lysis as provided herein does not require purification or extraction steps typically required or utilized to remove detergents. Rather, enzymes or other agents (e.g., ligase, alternatively or additionally, one or more cleavage systems such as CRISPR/Cas, TALEN, Zinc Finger, Restriction Enzyme, etc., and/or one or more hybridization reagents such as oligonucleotides—e.g., probes or primers, etc.) can be added directly to the lysis reaction (e.g., simultaneously or sequentially with any neutralization buffer).

Thus, the present disclosure provides lysis technologies that are remarkably compatible with nucleic acid processing and/among other things, in some embodiments may permit so-called “one pot” assessments.

### Example 2: Amplification of Dual-Epitope Cassette (Mock Trigger) with Different SDA Primers

This example demonstrates amplification of a DNA cassette with different SDA primers.

Amplification of Dual-Epitope cassette (Mock Trigger, TGAGGAGGAGTAATACGACTCACTATAGGGCATAAGACAAATACGACTCTGTCAG AGAGAATTAAGTAAGGAGGTTTTTTATGGATTACAAAGACCACGATGGTGACTAC AAAGACCATGATATAGATTACAAGGATGATGATGATAAATTTTTAAGTGTGTCACT TAACATTTGTACATGGTCACATCCTCAATTCGAGAAGTAATAATCTCCTCCTCAGC GATCTTCGACCTTC (SEQ ID NO: 17) with different SDA Primers. Amplification reactions contained 1× Custmart, 100 ng/uL T4gp32 (single stranded binding protein), 0.5 mM dNTPs, and either 0.25 U/uL Bsu DNAP and 0.5 U/uL Nb.BbvCI Nickase (Low), or 0.5 U/uL Bsu DNAP and 1 U/uL Nb.BbvCI Nickase (High).

Different SDA Primers were Added at 1 uM:

| | Standard unblocked: |
|---|---|
| (SEQ ID NO: 21) | |
| | GAAGGTCGAAGATCGCTGAGGAGGAG, |
| | Shorter unblocked: |
| (SEQ ID NO: 22) | |
| | GGGATCGCTGAGGAGGAG, |
| | and |
| | Standard Blocked: |
| (SEQ ID NO: 23) | |
| | GAAGGTCGAAGATCGCTGAGGAGGAG |
| | blocked with 3C6. |

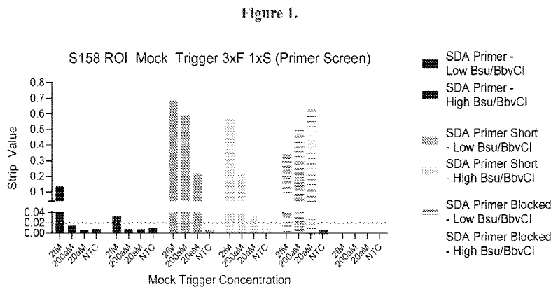

Expression cassette was amplified for 2 hours, followed by 2 hours of expression using NEBExpress kit, and detection of 3×FLAG-1×StrepII dual epitope expression cassette on lateral flow. The results show that unblocked short primers result in amplification, whereas primers having a blocking group provided better performance even with a longer primer (FIG. 1).

### Example 3: Amplification of Dual-Epitope Cassettes of Different Lengths

This example demonstrates amplification of DNA cassettes of different lengths. Reactions contained 1× Custmart, 100 ng/uL T4gp32, 0.5 mM dNTPs, 0.25 U/uL Bsu DNAP, 0.5 U/uL Nb.BbvCI Nickase, and 1 uM of short SDA Primer (SEQ ID NO: 22).

Different Expression were Added at Indicated Concentrations:

(386 bp)
(SEQ ID NO: 17)
TGAGGAGGAGTAATACGACTCACTATAGGGCATAAGACAAATACGACTCT
GTCAGAGAGAATTAAGTAAGGAGGTTTTTTATGGATTACAAAGACCACGA
TGGTGACTACAAAGACCATGATATAGATTACAAGGATGATGATGATAAAT
TTTTAAGTGTGTCACTTAACATTTGTACATGGTCACATCCTCAATTCGAG
AAGTAATAATCTCCTCCTCAGCGATCTTCGACCTTC,
(220 bp)
(SEQ ID NO: 18)
TGAGGAGGAGTAATACGACTCACTATAGGGCATAAGACAAATACGACTCT
GTCAGAGAGAATTAAGTAAGGAGGTTTTTTATGGATTACAAAGACCACGA
TGGTGACTACAAAGACCATGATATAGATTACAAGGATGATGATGATAAAT
TTTTAAGTGTGTCACTTAACATTTGTACAAGTGCGTGGTCACATCCTCAA
TTCGAGAAGGGCGGCGGGAGTGGAGGTGGCTCCGGTGGATCTGCTTGGTC
ACACCCCCAATTCGAAAAATAATAATCTCCTCCTCAGCGATCTTCGACCT
TC,
(244 bp)
(SEQ ID NO: 19)
TGAGGAGGAGTAATACGACTCACTATAGGGCATAAGACAAATACGACTCT
GTCAGAGAGAATTAAGTAAGGAGGTTTTTTATGGATTACAAGGATGATGA
TGATAAATTTTTAAGTGTGTCACTTAACATTTGTACATGGTCACATCCTC
AATTCGAGAAGTAATAATCTCCTCCTCAGCGATCTTCGACCTTC,
and
(178 bp)
(SEQ ID NO: 20)
TGAGGAGGAGTAATACGACTCACTATAGGGCATAAGACAAATACGACTCT
GTCAGAGAGAATTAAGTAAGGAGGTTTTTTATGGATTACAAGGATGATGA
TGATAAATTTTTAAGTGTGTCACTTAACATTTGTACAAGTGCGTGGTCAC
ATCCTCAATTCGAGAAGGGCGGCGGGAGTGGAGGTGGCTCCGGTGGATCT
GCTTGGTCACACCCCCAATTCGAAAAATAATAATCTCCTCCTCAGCGATC
TTCGACCTTC.

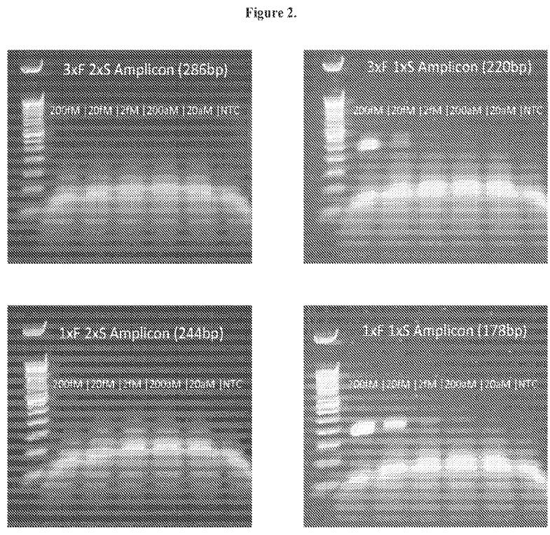

Cassette starting material was amplified for 2 hours, followed by gel electrophoresis of products on a 2% B-Gel. 3xF 1xS (SEQ ID NO 18) and 1xF 1xS (SEQ ID NO: 20) having a nucleotide length of 220 bp and 178 bp respectively, were amplified and detected, using various SDA primer concentrations (FIG. 2).

### Example 4: Amplification of Dual-Epitope Cassettes of Different Lengths

This example demonstrates Amplification of Dual-Epitope cassettes of different lengths.

Reactions contained 1× Custmart, 100 ng/uL T4gp32, 0.5 mM dNTPs, 0.25 U/uL Bsu DNAP, 0.5 U/uL Nb.BbvCI Nickase, and 1 uM of short SDA Primer (SEQ ID NO: 22). Different expression were added at indicated concentrations
SEQ ID NO: 17 (386 bp),
SEQ ID NO: 18 (220 bp),
SEQ ID NO: 19 (244 bp), and
SEQ ID NO: 20 (178 bp).

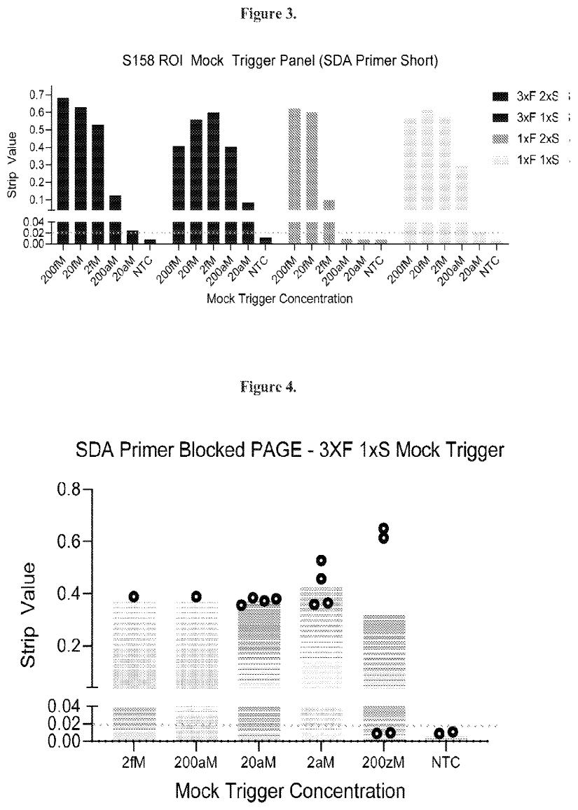

Cassette was amplified for 2 hours, followed by 2 hours of expression using NEBExpress kit, and detection of dual epitope peptides on lateral flow. Shorter cassette length and higher SDA primer concentration resulted in improved amplification compared to longer cassettes and lower SDA primer concentration (FIG. 3).

### Example 5: Lateral Flow LOD of a Cassette Using Cap-SDA

This example demonstrates lateral Flow LOD of a cassette using Cap-SDA.

Reactions contained 1× Custmart, 100 ng/uL T4gp32, 0.5 mM dNTPs, 0.25 U/uL Bsu DNAP, 0.5 U/uL Nb.BbvCI Nickase, and 1 uM of blocked SDA Primer (SEQ ID NO: 23 blocked with 3C6). A 3×FLAG-1×StrepII cassette (SEQ ID NO: 17) was added at indicated concentrations (2 fM, 200 aM, 20 aM, 2 aM, and 200 zM). Cassette was amplified for 2 hours, followed by 2 hours of expression using NEBExpress kit, and detection of dual epitope peptides on lateral flow.

Cassettes were amplified, expressed and detected at all cassette concentrations (ranging from 2 fM to 200 zM). The present example demonstrated ability to detect a cassette when present in the sample at a very low concentration (FIG. 4).

### Example 5: Ligation Reaction Followed by Amplification Using Single Stranded Binding Protein

This example demonstrates reduction of NTC background signal from a ligation reaction amplified by Cap-SDA using an extremely thermostable (ET)-SSB.

Ligation reactions contained 1×T4 ligase buffer, 2 nM probe mix (TGAGGAGGAGTAATACGACTCACTATAGGGGCACAATCACCAA (SEQ ID NO: 24), AAGATCTGAATCGTGGTCACATCCTCAATTCGAGAAGTAATAATCTCCTCCTCA (SEQ ID NO: 25) comprising a 5PHOS, TCAAAGTTGAATCTGCATAAGGAGGTTTTTTATGGATTACAAAGACCACGATGGT GACTACAAAGACCATGATATAGATTACAAGGATGATGATGATAAATCAGAGACAA AGTCATT (SEQ ID NO: 26) comprising a 5PHOS), 500 nM SplintR Ligase, and if indicated 10% PEG-3350 and/or 25 ng/uL ET-SSB. After 30 minutes these reactions were then amplified by Cap-SDA. Cap-SDA reactions contained 1× Custmart, 100 ng/uL T4gp32, 0.5 mM dNTPs, 0.25 U/uL Bsu DNAP, 0.5 U/uL Nb.BbvCI Nickase, and 1 uM of blocked SDA Primer (SEQ ID NO: 23 blocked with 3C6). Ligation reactions were amplified for 2 hours, followed by 2 hours of expression using NEBExpress kit, and detection of dual epitope peptides on lateral flow.

Using a single stranded binding protein (ET-SSB) during amplification reduce NTC background signal (FIG. 5).

### Example 7: Ligation Reaction Followed by Amplification

This example demonstrates reduction of NTC background signal from a ligation reaction amplified by Cap-SDA using an A probe comprising a Ribosome Binding Site (RBS) (New Design).

Ligation reactions contained 1×T4 ligase buffer, 2 nM probe mix (SEQ ID NO: 24, SEQ ID NO: 25 comprising a 5PHOS, SEQ ID NO: 26 comprising a 5PHOS 10 for Older Design and TGAGGAGGAGTAATACGACTCACTATAGGGGATTAAGTAAGGAGGTTTTTTATGG CACAATCACCAA (SEQ ID NO: 27), AAGATCTGAATCGTGGTCACATCCTCAATTCGAGAAGTAATAATCTCCTCCTCA (SEQ ID NO: 28) comprising a 5PHOS, TCAAAGTTGAATCTGCAGATTACAAAGACCACGATGGTGACTACAAAGACCATG ATATAGATTACAAGGATGATGATGATAAATCAGAGACAAAGTCATT (SEQ ID NO: 29) comprising a 5PHOS for New Design), 500 nM SplintR Ligase, and if indicated 10% PEG-3350 and/or 25 ng/uL ET-SSB. After 30 minutes these reactions were then amplified by Cap-SDA. Cap-SDA reactions contained 1× Custmart, 100 ng/uL T4gp32, 0.5 mM dNTPs, 0.25 U/uL Bsu DNAP, 0.5 U/uL Nb.BbvCI Nickase, and 1 uM of blocked SDA Primer (SEQ ID NO: 23 blocked with 3C6). Ligation reactions were amplified for 2 hours, followed by 2 hours of expression using NEBExpress kit, and detection of dual epitope peptides on lateral flow.

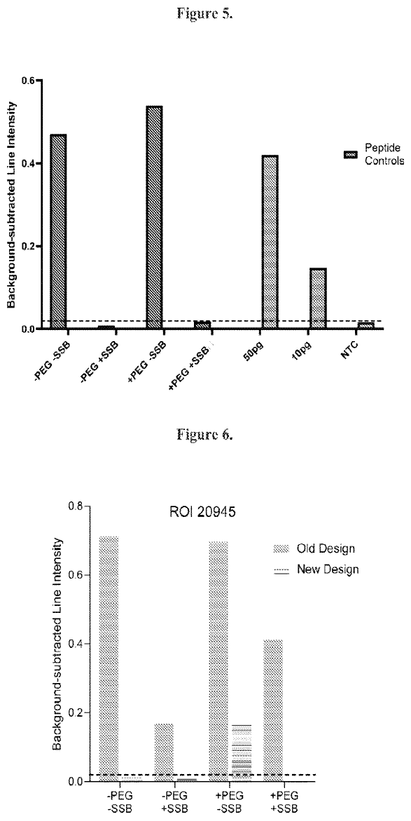

The Example shows that a probe deign (Old Design) incorporating RBS into the GFO resulted in significant background signal in all combinations of PEG and/or SSB tested in the ligation step, whereas a probe design (New Design) incorporating the RBS into the A probe resulted in low background, particularly in the presence of SSB (FIG. 6).

### Example 8: Ligation Followed by Amplification

This example demonstrates detection of SARS gRNA target in a ligation reaction amplified by Cap-SDA with different probe design.

Ligation reactions contained 1×T4 ligase buffer, 2 nM probe mixes (30 bp Standard—SEQ ID NO: 27, SEQ ID NO: 28 comprising a 5PHOS, SEQ ID NO: 29 comprising a 5PHOS; 40 bp Gap—TGAGGAGGAGTAATACGACTCACTATAGGGGATTAAGTAAGGAGGTTTTTTATGT TATTAGCTGTATGT (SEQ ID NO: 30), CAGCGTACCCGTAGGCTGGTCACATCCTCAATTCGAGAAGTAATAATCTCCTCCTC A (SEQ ID NO: 31) comprising a 5PHOS, ACAGTTGCACAATCAGATTACAAAGACCACGATGGTGACTACAAAGACCATGATA TAGATTACAAGGATGATGATGATAAATCTGAATCGACAAG (SEQ ID NO: 32) comprising a 5PHOS; 51 bp HybSeqs Equal Length—TGAGGAGGAGTAATACGACTCACTATAGGGGATTAAGTAAGGAGGTTTTTTATGT TATTAGCTGTATGTACAGTTGCACA (SEQ ID NO: 33), ATCGACAAGCAGCGTACCCGTAGGCTGGTCACATCCTCAATTCGAGAAGTAATAA TCTCCTCCTCA (SEQ ID NO: 34) comprising a 5PHOS, ATCACCAATCAAAGTTGAATCTGCAGATTACAAAGACCACGATGGTGACTACAAA GACCATGATATAGATTACAAGGATGATGATGATAAATCAGAGACAAAGTCATTAAG ATCTGA (SEQ ID NO: 35) comprising a 5PHOS; 51 bp HybSeqs—TGAGGAGGAGTAATACGACTCACTATAGGGGATTAAGTAAGGAGGTTTTTTATGT TATTAGCTGTATGTACAGTTGCACAATCACCAA (SEQ ID NO: 36), AAGATCTGAATCGACAAGCAGCGTACCCGTAGGCTGGTCACATCCTCAATTCGAG AAGTAATAATCTCCTCCTCA (SEQ ID NO: 37) comprising a 5PHOS, TCAAAGTTGAATCTGCAGATTACAAAGACCACGATGGTGACTACAAAGACCATG ATATAGATTACAAGGATGATGATGATAAATCAGAGACAAAGTCATT (SEQ ID NO: 38) comprising a 5PHOS), 500 nM SplintR Ligase, 10% PEG-3350, and 25 ng/uL ET-SSB. After 30 minutes these reactions were then amplified by Cap-SDA. Cap-SDA reactions contained 1× Custmart, 100 ng/uL T4gp32, 0.5 mM dNTPs, 0.25 U/uL Bsu DNAP, 0.5 U/uL Nb.BbvCI Nickase, and 1 uM of blocked SDA Primer (SEQ ID NO: 23 blocked with 3C6). Ligation reactions were amplified for 2 hours, followed by 2 hours of expression using NEBExpress kit, and detection of dual epitope peptides on lateral flow.

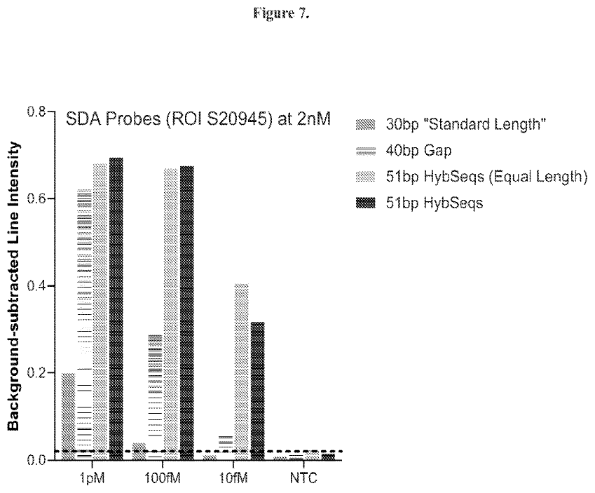

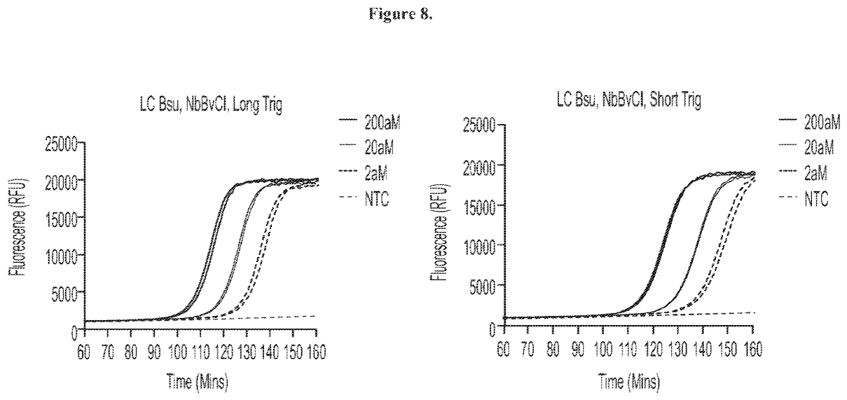

FIG. 7 shows that introducing a gap between the two hybridization regions using longer hybridization lengths improve signal intensity (FIG. 7).

### Example 9: Detection of Synthetic ssDNA Targets

This example demonstrates detection of synthetic ssDNA targets (DNA cassette) containing ORF1 SARS sequence (Long Trig—TGAGGAGGAGATGTTCAACAATGGGGTTTTACAGGTAACCTACAAAGCAACCAT GATCTGTATTGTCAAGTCCACTCCTCCTCA (SEQ ID NO: 40), Short Trig—TGAGGAGGAGATACAGATCATGGTTGCTTTGTAGGTTACCTGTAAAACCTCCTCC TCA (SEQ ID NO: 39)).

Cap-SDA reactions contained 1× Custmart, 100 ng/uL T4gp32, 0.5 mM dNTPs, 1 mM rNTPs, 2.5 mM DTT, 5 U/uL T7 RNAP, 200 nM RNAse Alert, 1 uM of blocked SDA Primer (SEQ ID NO: 23 blocked with 3C6), 100 nM of blocked T7-SDA primer (TAATACGACTCACTATAGGGCTGAGGAGGAG (SEQ ID NO: 41) blocked with 3C6), and 10 nM Cas13/crRNA complex (GAUUUAGACUACCCCAAAAACGAAGGGGACUAAAACGAUCAUGGUUGCUUU GUAGGUUACCUGU (SEQ ID NO: 69)). They also contained either 0.5 U/uL Bsu DNAP and 1 U/uL Nb.BbvCI Nickase from low concentration (LC) stocks (5 U/uL Bsu, 10 U/uL Nb.BbvCI), or the same amount of polymerase and nickase from high concentration (HC) stocks (50 U/uL Bsu, 400 U/uL Nb.BbvCI), or with a high concentration IsoPol DNAP stock instead of Bsu (35 U/uL Isopol). Detection was carried out in real time by measuring fluorescence on a plate reader.

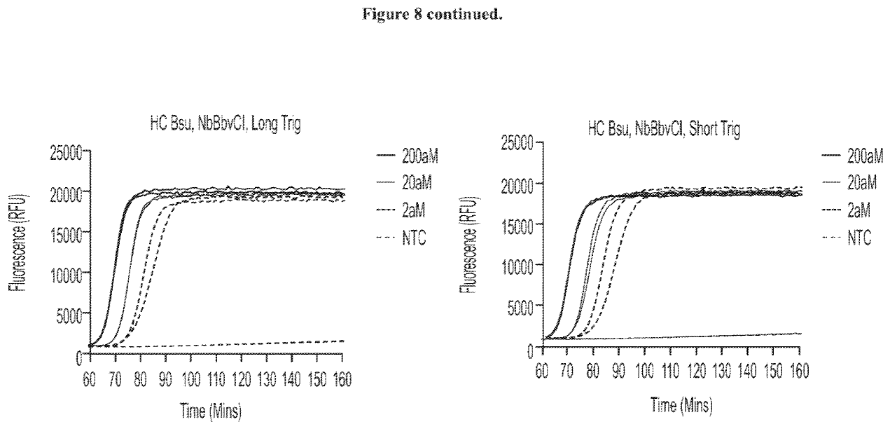

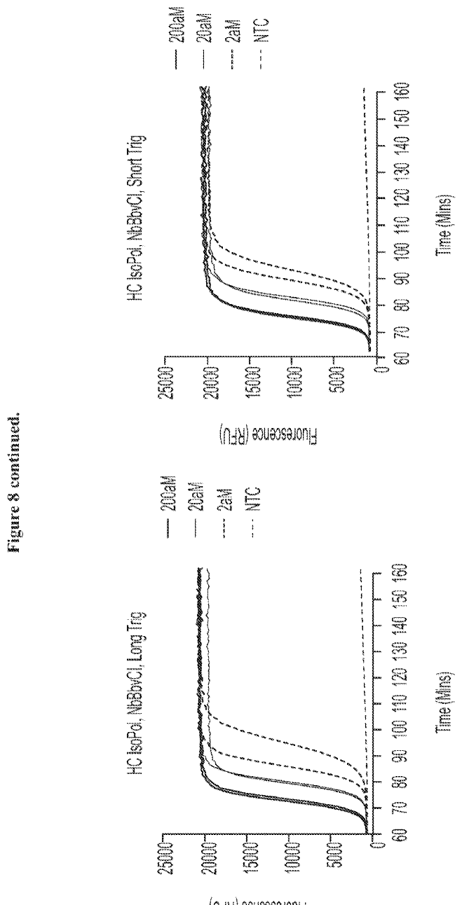

The Example shows that using LC enzyme stocks (5 U/uL Bsu, 10 U/uL Nb.BbvCI) in combination with high levels of glycerol did not lead to a reduction in performance compared to HC enzyme stocks (50 U/uL Bsu, 400 U/uL Nb.BbvCI) (FIG. 8).

### Example 10: Detection of Synthetic ssDNA Targets

This example demonstrates detection of synthetic ssDNA targets (DNA cassette) containing ORF1 SARS sequence (Short Trig—SEQ ID NO: 39) with and without treatment with Klenow Fragment (KF).

Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI). Additionally either 1× EvaGreen dye was added, or 200 nM RNAse Alert with 10 nM Cas13/crRNA complex (Seq 53) was added. Detection was carried out in real time by measuring fluorescence on a plate reader. Blocked SDA Primers had been beforehand either untreated (suspended in water) or KF-treated (incubated with 0.5 U/uL Klenow Fragment in Tris buffer with 10 mM MgCl2 and no dNTPS for 1 hour at 37 C, followed by 20 min heat kill at 85 C). The 3′ exo activity of KF enriches for only blocked primers and cleave any unblocked primers.

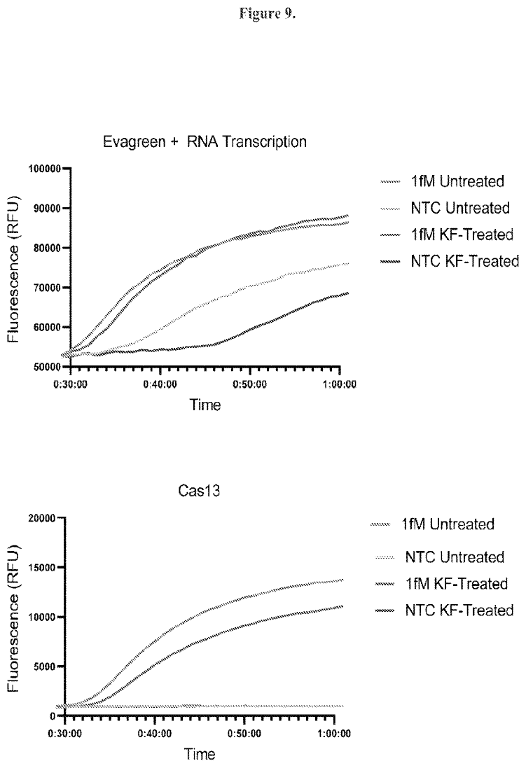

The Example shows that a KF-treatment of the SDA primer resulted in a delay of the onset of the NTC curve, without delaying the onset of the Target curve (either measured by Evagreen dsDNA dye, or by Cas13 output) (FIG. 9).

### Example 11: Reverse Transcriptase and Ligase Reaction Followed by Amplification

This example demonstrates detection of SARS irradiated viral particles using either a Reverse Transcriptase (A) or a Ligase reaction (B), amplified subsequently by Cap-SDA.

Prior to detection encapsulated SARS virus was lysed using SARSBEGONE reagent (0.1% LAPAO and 10 mM HCL, neutralized with 100 mM Tris pH 9). Reverse Transcriptase reactions contained 1× Protoscript II buffer, 10 mM DTT, 4 U/uL Protoscript II RT Pol, 25 ng/uL ET-SSB, 0.1 U/uL RNAse H, 0.5 mM dNTPs, and 20 nM of F and R primers (TGAGGAGGAGATGTTCAACAATGGG (SEQ ID NO: 42) and CCGATCGCTGAGGAGGAGTGGACTTGACAA (SEQ ID NO: 46)). Ligation reactions contained 1×T4 ligase buffer, 2 nM probe mix (TGAGGAGGAGGACAATACAGATCA (SEQ ID NO: 56), TGGTTGCTTTGT (SEQ ID NO: 57) comprising a 5PHOS, AGGTTACCTGTAAACTCCTCCTCA (SEQ ID NO: 58) comprising a 5PHOS), 500 nM SplintR Ligase, 10% PEG-3350, and 25 ng/uL ET-SSB. Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM RNAse Alert, and 10 nM Cas13/crRNA complex (SEQ ID NO: 69). Detection was carried out in real time by measuring fluorescence on a plate reader.

The data show that both the RT polymerase and SplintR Ligation based workflows are compatible with the SARSBEGONE lysis reagent (FIG. 10).

### Example 12: Reverse Transcriptase Reaction Followed by Amplification

This example demonstrates detection of SARS gRNA using a Reverse Transcriptase, amplified subsequently by Cap-SDA.

Reverse Transcriptase reactions contained 1× Protoscript II buffer, 10 mM DTT, 4 U/uL Protoscript II RT Pol, 25 ng/uL ET-SSB, 0.1 U/uL RNAse H, 0.5 mM dNTPs, and indicated RT primers (paired F and Rv2 designs, SEQ ID NO: 42, TGAGGAGGAGATGTTCAACAAT (SEQ ID NO: 43), TGAGGAGGAGATGTTCAACA (SEQ ID NO: 44), CCGATCGCTGAGGAGGAGTGGACTTGACAATAC (SEQ ID NO: 45), SEQ ID NO: 46, and CCGATCGCTGAGGAGGAGTGGACTTGAC (SEQ ID NO: 47)).

Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM RNAse Alert, and 10 nM Cas13/crRNA complex (SEQ ID NO: 69). Detection was carried out in real time by measuring fluorescence on a plate reader, and the endpoint signal at 120 or 150 minutes is displayed.

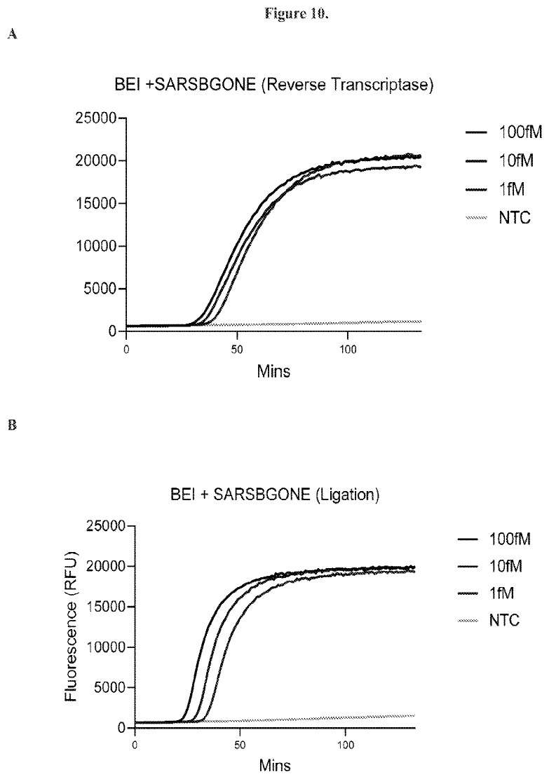

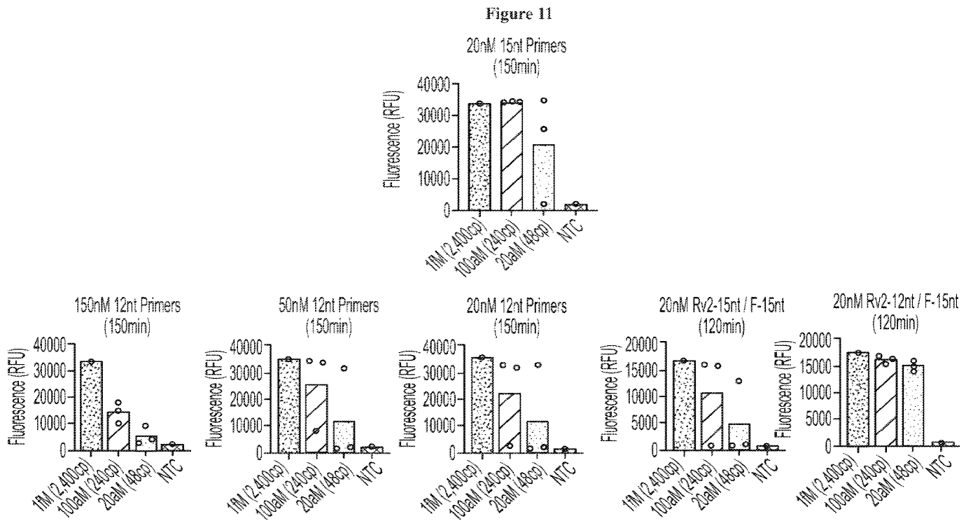

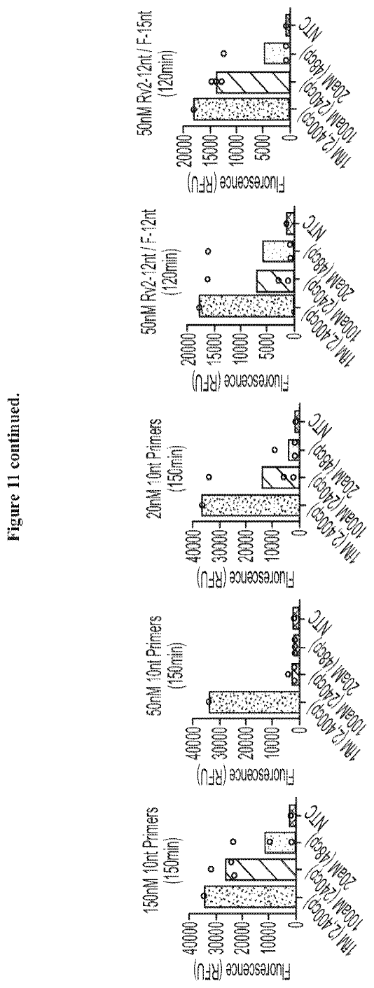

Results demonstrate that a range of hybridization lengths (10 nt-15 nt primers) and primer concentrations (20 aM to 1M) for reverse transcriptase primers can be conducive to signal detection (FIG. 11).

### Example 13: Reverse Transcriptase Reaction Including Bump Primer Followed by Amplification

This example demonstrates detection of SARS gRNA using a Reverse Transcriptase, amplified subsequently by Cap-SDA.

Reverse Transcriptase reactions contained 1× Protoscript II buffer, 10 mM DTT, 4 U/uL Protoscript II RT Pol, 25 ng/uL ET-SSB, 0.1 U/uL RNAse H, 0.5 mM dNTPs, and indicated RT primers including a bump primer (SEQ ID NO: 42, TGAGGAGGAGTGGACTTGACAATAC (SEQ ID NO: 48), and ATTACGTCTATAATC (SEQ ID NO: 49)). Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM RNAse Alert, and 10 nM Cas13/crRNA complex (SEQ ID NO: 69). Detection was carried out in real time by measuring fluorescence on a plate reader.

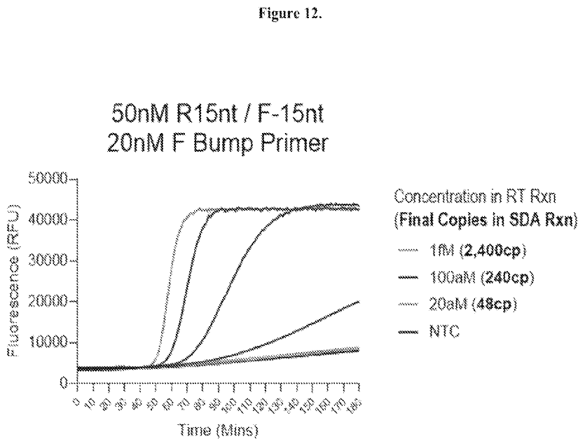

Results demonstrate that the RT-SDA workflow can also be used with the addition of a bump primer (FIG. 12).

### Example 14: Reverse Transcriptase Reaction Followed by Amplification

This example demonstrates detection of SARS gRNA using a Reverse Transcriptase, amplified subsequently by Cap-SDA.

Reverse Transcriptase reactions contained 1× Protoscript II buffer, 10 mM DTT, 4 U/uL Protoscript II RT Pol, 25 ng/uL ET-SSB, 0.1 U/uL RNAse H, 0.5 mM dNTPs, and indicated RT primers (SEQ ID NO: 42 and SEQ ID NO: 46). Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM RNAse Alert, and 10 nM Cas13/crRNA complex (SEQ ID NO: 69). Additionally, the Cap-SDA reactions contained either no Klenow LF, or 0.0125 U/uL Klenow LE Detection was carried out in real time by measuring fluorescence on a plate reader.

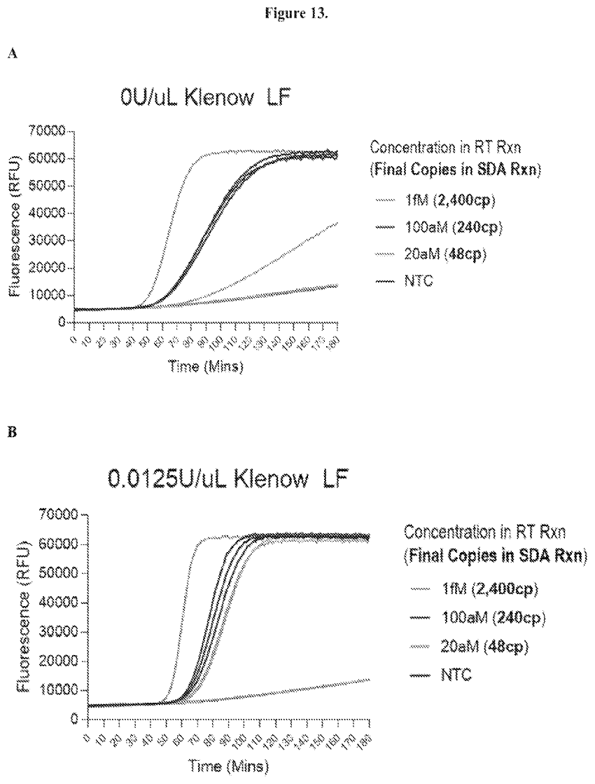

FIG. 13 demonstrates that sensitivity of the RT-SDA workflow is enhanced when adding a low concentration of Klenow Large Fragment DNA polymerase to the SDA reaction (FIG. 13).

### Example 15: Ligase Reaction Followed by Amplification

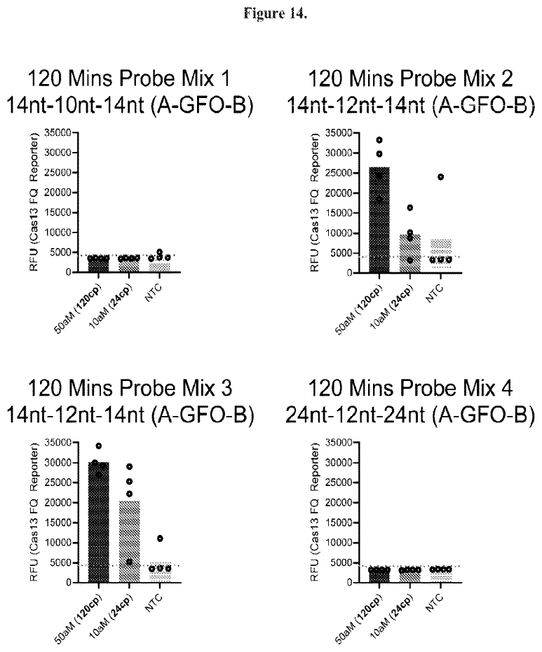

This example demonstrates detection of SARS gRNA using a Ligase reaction (using probes having different hybridization lengths as indicated in FIG. 14), amplified subsequently by Cap-SDA.

Ligation reactions contained 1×T4 ligase buffer, 2 nM probe mixes (Probe Mix 1—TGAGGAGGAGATACAGATCATGGT (SEQ ID NO: 50), TGCTTTGTAG (SEQ ID NO: 51) comprising a 5PHOS, GTTACCTGTAAAACCTCCTCCTCA (SEQ ID NO: 52) comprising a 5PHOS 34, 35, 36; Probe Mix 2—TGAGGAGGAGATACAGATCATGGT (SEQ ID NO: 53), TGCTTTGTAGGT (SEQ ID NO: 54) comprising a 5PHOS, TACCTGTAAAACCCCTCCTCCTCA (SEQ ID NO: 55) comprising a 5PHOS37, 38, 39; Probe Mix 3—SEQ ID NO: 56, SEQ ID NO: 57 comprising a 5PHOS, SEQ ID NO: 58 comprising a 5PHOS; Probe Mix 4—TGAGGAGGAGCCATGGACTTGACAATACAGATCA (SEQ ID NO: 59), TGGTTGCTTTGT (SEQ ID NO: 60) comprising a 5PHOS, AGGTTACCTGTAAAACCCCATTGTCTCCTCCTCA (SEQ ID NO: 61) comprising a 5PHOS), 500 nM SplintR Ligase, 10% PEG-3350, and 25 ng/uL ET-SSB. Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM RNAse Alert, and 10 nM Cas13/crRNA complex (SEQ ID NO: 69). Detection was carried out in real time by measuring fluorescence on a plate reader, and the signal at 120 minute timepoint is displayed.

The results show that Probe Mix 1 using short probes (14 nt) and short GFO and Probe Mix 4 using long probes (24 nt) result in lost sensitivity, i.e., the ability to detect a target nucleic acid sequence (FIG. 14).

### Example 16: Detection of ssDNA Synthetic Target

This example demonstrates detection of 2 fM of ssDNA synthetic target containing the SARS ORF1 sequence by Cap-SDA

(SEQ ID NO: 68)
(TGAGGAGGAGGACAATACAGATCATGGTTGCTTTGTAGGTTACCTGTAA
ACTCCTCCTCA).

Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), and 10 nM Cas13/crRNA complex (SEQ ID NO: 69). Additionally, either 200 nM or 8 uM of a fluorophore-quencher reporter was included (RURURURURURURURURURURURU (SEQ ID NO: 62) comprising a 56FAM and a 3BQH1, RURURURURU (SEQ ID NO: 63) comprising a 56FAM and a 3BQH1, ATRURUGC (SEQ ID NO: 64) comprising a 5HEX and a 3IWBK). Detection was carried out in real time by measuring fluorescence on a plate reader.

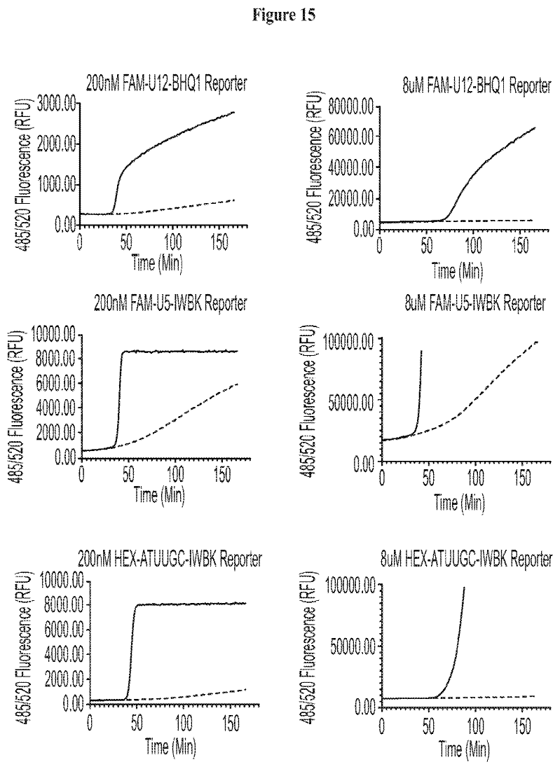

Results show that both FAM and HEX can be used in a Cas13 cleavage reporter, and that shorter sequences in the cleavage reporter result in faster time to result. (FIG. 15).

### Example 17: Ligation Reaction Followed by Amplification

This example demonstrates colorimetric detection of SARS gRNA at indicated concentrations using a Ligase reaction amplified subsequently by Cap-SDA.

Ligation reactions contained 1×T4 ligase buffer, 2 nM probe mixes, 500 nM SplintR Ligase, 10% PEG-3350, and 25 ng/uL ET-SSB. Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 20 uM FQ reporter (SEQ ID NO: 63 comprising a 56FAM and a 3BQH1), and 10 nM Cas13/crRNA complex (SEQ ID NO: 69). Detection was carried out in real time by observing color change, and a photo was taken at 90 minutes.

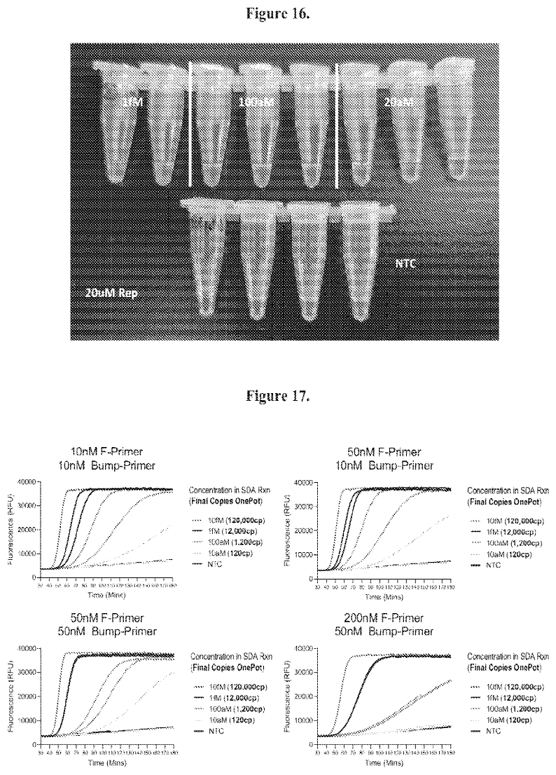

Results show a visible color change occur upon reporter activation using fluorescence-quencher cleavage reporter concentrations of 20 aM-1 fM which was not observed for the control samples (FIG. 16).

### Example 18: Amplification of dsDNA Containing Native Nickase Site

This example demonstrates detection of a dsDNA target containing a native nickase site (CCGATCGCTGAGGAGGAGTGGACTTGACAATACAGATCATGGTTGCTTTGTAGG TTACCTGTAAAACCCCATTGTTGAACATCAATCATAAACGGATTATAGACGTAATC AAATCCAATAGA (SEQ ID NO: 65)) in a one-pot SDA reaction.

Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM FQ reporter (SEQ ID NO: 63 comprising a 5′6FAM and a 3′BQH1), and 10 nM Cas13/crRNA complex (SEQ ID NO: 69). Additionally, a F primer and F bump primer were added (SEQ ID NO: 42 and SEQ ID NO: 49). Detection was carried out in real time by measuring fluorescence on a plate reader.

Results demonstrate one-pot detection of a dsDNA target (FIG. 17). Bump primers were used to make the DNA strand generated by the opposing primer single-stranded after priming and extension.

### Example 19: Lateral Flow Detection of ssDNA

This example demonstrates detection of a ssDNA synthetic target (SEQ ID NO: 68, − indicates 0 fM, + indicates 20 fM) with lateral flow output form a Cap-SDA reaction using capture oligos.

Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI). Additionally, the indicated concentration of a Biotin-probe and FAM-probe were added (TACCTGTAAACTCCTCCTCA (SEQ ID NO: 66) comprising a 3′BIOTEG and ATCATGGTTGCTTTGTAGGT (SEQ ID NO: 67) comprising a 5′6-FAM and a 3C6). Detection was carried out after two hours with a biotin-FAM lateral flow strip.

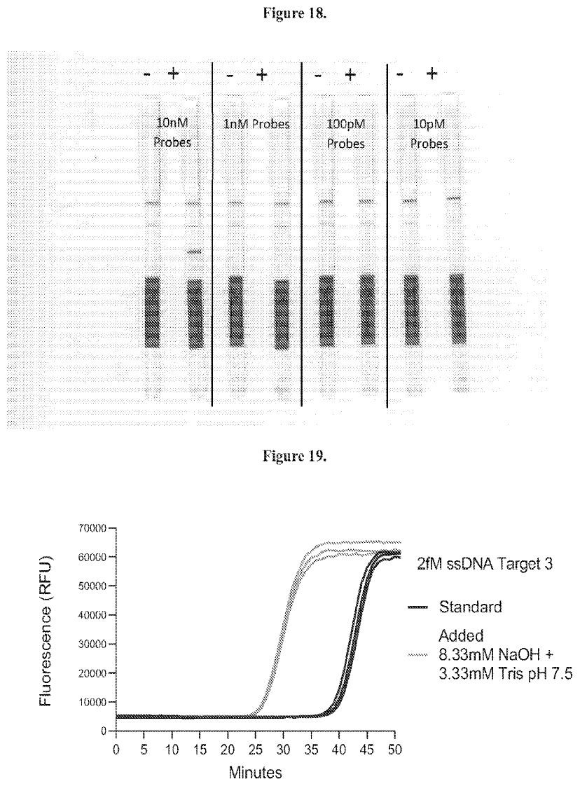

Results demonstrate detection of SDA-amplified cassette using two hybridization probes and a lateral flow strip (FIG. 18).

### Example 20: Detection of ssDNA

This example demonstrates detection of a ssDNA synthetic target (2 fM, SEQ ID NO: 68) with a Cap-SDA reaction under standard or pH-adjusted conditions.

Cap-SDA reactions contained 1× Custmart, 100 ng/uL T4gp32, 0.5 mM dNTPs, 1 mM rNTPs, 2.5 mM DTT, 5 U/uL T7 RNAP, 1 uM of blocked SDA Primer (SEQ ID NO: 23 blocked with 3C6), 100 nM of blocked T7-SDA primer (SEQ ID NO: 41 blocked with 3C6), 0.5 U/uL Bsu DNAP, 1 U/uL Nb.BbvCI Nickase from high concentration stocks (50 U/uL Bsu, 400 U/uL Nb.BbvCI), 200 nM of FQ reporter (SEQ ID NO: 63 comprising a 5′6FAM and a 3′BQH1), and 10 nM of Cas13/crRNA complex (SEQ ID NO: 69). Additionally 8.3 mM NaOH and 3.33 mM Tris pH 7.5, or nothing was added. Detection was carried out in real time by measuring fluorescence on a plate reader.

Results show that the Cap-SDA reaction using higher pH than standard resulted in faster detection of the ssDNA target (FIG. 19).

### Example 21: Amplification Using Nb.BbvCI Endogenous Nick

This example demonstrates detection of a dsDNA genomic target (Ct extracted genome) using an Nb.BbvCI endogenous nick site with a Cap-SDA reaction without the use of a bump primer (only using an opposing primer).

Cap-SDA reactions contained 100 mM Tris pH 7, 10 mM KOAc, 100 ng/uL T4gp32, 0.5 mM dNTPs, 1 mM rNTPs, 2.5 mM DTT, 5 U/uL T7 RNAP, 1 uM of blocked SDA Primer (having a length of 25 nucleotides and blocked with 3C6), 100 nM of blocked T7-SDA primer (having a length of 30 nucleotides and blocked with 3C6), 100 nM of an 22 nucleotide opposing oligo, 0.5 U/uL Bsu DNAP, 1 U/uL Nb.BbvCI Nickase from high concentration stocks (50 U/uL Bsu, 400 U/uL Nb.BbvCI), 200 nM of FQ reporter (rUrUrUrUrU (SEQ ID NO: 2) comprising a 5′6FAM fluorophore and 3′BQH1 quencher), and 10 nM of Cas13/crRNA complex (GAUUUAGACUACCCCAAAAACGAAGGGGACUAAAACUUAGUUCCUAGGUAC UAUACGUUAUGUC (SEQ ID NO: 1)). The target dsDNA was denatured in a mix of 80 mM KOH and 30 mM MgOAc, then added to the reaction to initiate it (final KOH was 40 mM and final MgOAc was 15 mM). Reaction was run at room temperature.

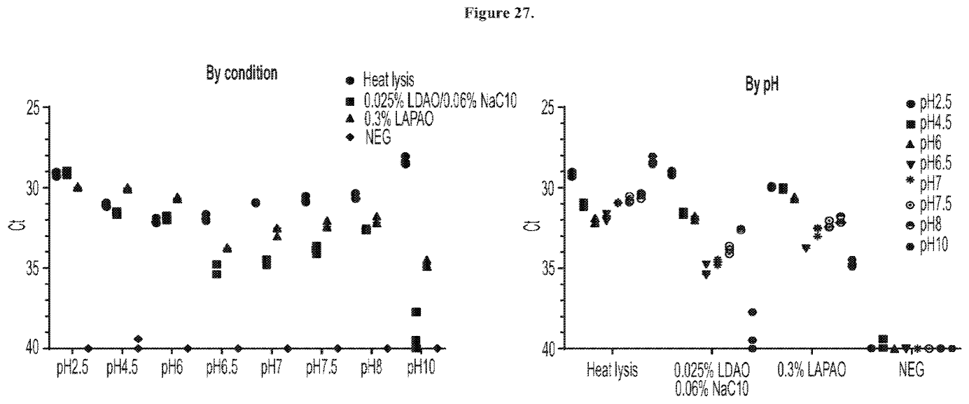

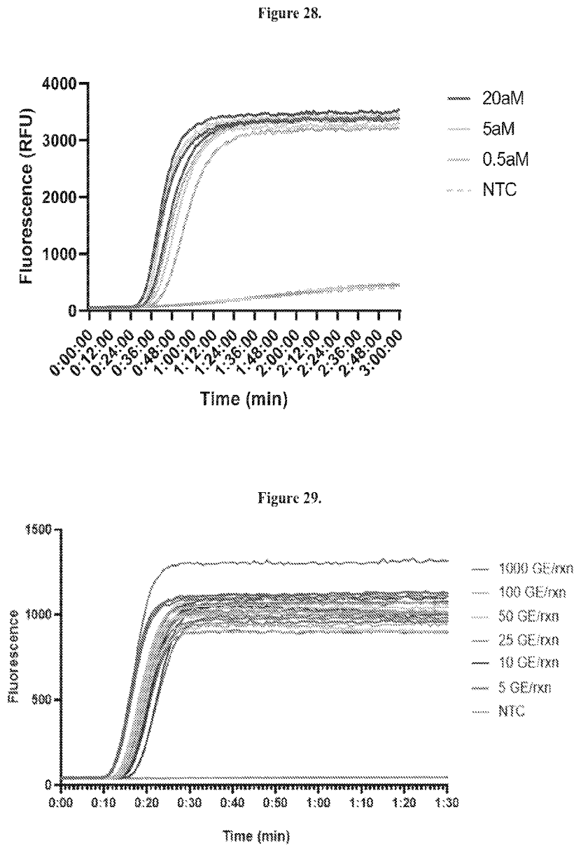

The example shows dsDNA amplification with the Nb.BbvCI nickase using a single endogenous nick site without the use of a bump primer (FIG. 28). This Example demonstrates a detection method that eliminates the need of designing an additional primer (FIG. 28), and was also shown to improve speed and sensitivity of the assay compared with the workflow that does use a bump primer (FIG. 17).

### Example 22: Amplification Using a Nickase Nt.CviPII and LwCas13a

This example demonstrates detection of a dsDNA genomic target (Ng extracted genome) with a Cap-SDA reaction with the nickase Nt.CviPII and LwCas13a, using short endogenous CCD nickase recognition sites.

Cap-SDA reactions contained 50 mM Tris pH 8.75, 50 mM KOAc, 200 ng/uL T4gp32, 0.5 mM dNTPs, 0.5 mM rNTPs, 2.5 mM DTT, 2 U/uL T7 RNAP, 250 nM each of blocked SDA primers (both having a length of 29 nucleotides and blocked with 3C6), 200 nM of blocked T7-SDA primer (having a length of 28 nucleotides and blocked with 3C6), 0.5 U/uL Bsu DNAP, 0.05 U/uL Nt.CviPII Nickase from high concentration stocks (165 U/uL Bsu, 20 U/uL Nt.CviPII), 200 nM of FQ reporter (rUrUrUrUrU (SEQ ID NO: 2) comprising a 5′6FAM fluorophore and 3′BQH1 quencher), and 10 nM of Cas13/crRNA complex. The target dsDNA was diluted in 30 mM MgOAc, then added to the reaction to initiate it (final MgOAc was 15 mM). Reaction was run at room temperature.

This data demonstrates detection using an Nt.CviPII-based workflow targeting two opposing endogenous nick sites, and a Cas13 signal output. FIG. 29 shows high target sensitivity and detection within 10-15 minutes. Sensitivity and time to result are improved compared to Nb.BbvCI-based workflows (Time to result has gone from 25-90 min to 10-15 mins compared to FIG. 28).

### Example 23: Amplification Using a Nickase Nt.CviPII and LbCas12a

This example demonstrates detection of a dsDNA genomic target (Ng extracted genome) with a Cap-SDA reaction with the nickase Nt.CviPII and LbCas12a, using short endogenous CCD nickase recognition sites.

Cap-SDA reactions contained 50 mM Tris pH 8.75, 50 mM KOAc, 150 ng/uL T4gp32, 0.5 mM dNTPs, 2.5 mM DTT, 1 uM each of blocked SDA primers (TGCTGCAGCTAGAAGTCCGAGAAGT (SEQ ID NO: 3) blocked with 3C6 and TGCTGCAGCTAGAAGTCCGTAGACA (SEQ ID NO: 4) blocked with 3C6), 0.5 U/uL Bsu DNAP, 0.1 U/uL Nt.CviPII Nickase from high concentration stocks (165 U/uL Bsu, 20 U/uL Nt.CviPII), 200 nM of FQ reporter (TTATTTTATT (SEQ ID NO: 5) comprising a 56FAM fluorophore and a 3BQH1 quencher), and 10 nM of Cas12/gRNA complex (UAAUUUCUACUAAGUGUAGAUGAAGUGAUGACGAGUGUCUA (SEQ ID NO: 6)).

The target dsDNA was diluted in 30 mM MgOAc, then added to the reaction to initiate it (final MgOAc was 15 mM). Reaction was run at room temperature.

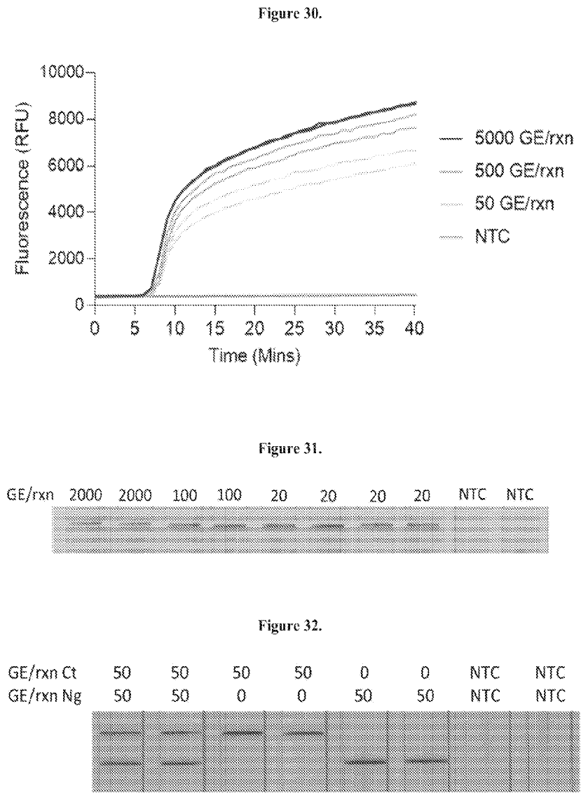

This data shows detection with an Nt.CviPII-based workflow targeting two opposing endogenous nick sites, and a Cas12 signal output. FIG. 30 shows that the target nucleic acid is detected within 8 minutes using this Nt.CviPII-based workflow.

### Example 24: Amplification Using a Nickase Nt.CviPII

This example demonstrates detection of a dsDNA genomic target (Ng extracted genome) with a Cap-SDA reaction with the nickase Nt.CviPII using short endogenous CCD nickase recognition sites with a direct oligo pulldown assay on a lateral flow strip.

Amplified material is detected by direct capture on a lateral flow strip (capture sequence 4×TGTA). Cap-SDA reactions contained 50 mM Tris pH 8.75, 50 mM KOAc, 200 ng/uL T4gp32, 0.5 mM dNTPs, 2.5 mM DTT, 250 nM each of blocked SDA primers (both having a length of 29 nucleotides and blocked with 3C6), 0.5 U/uL Bsu DNAP, 0.05 U/uL Nt.CviPII Nickase from high concentration stocks (165 U/uL Bsu, 20 U/uL Nt.CviPII), 250 nM of amplicon capture probes. The target dsDNA was diluted in 30 mM MgOAc, then added to the reaction to initiate it (final MgOAc was 15 mM). Reaction was run at room temperature for 30 minutes before running on the lateral flow strip. Running buffer for the lateral flow contained 50 mM Tris pH 8.75, 50 mM KOAc, 2% Ecosurf, and 2 uM of a Poly-T oligo to reduce background.

This data displays detection using cSDA amplification reactions with a direct oligo pulldown assay on a lateral flow strip. All reaction bands (200, 200, 100, 100, 20, 20, 20, 20) were visible on the lateral flow strip for (FIG. 31).

### Example 25: Amplification Using a Nickase Nt.CviPII Followed by Multiplex Detection

This example demonstrates multiplexed detection of two dsDNA genomic targets (either Ct or Ng extracted genome) with a Cap-SDA reaction with the nickase Nt.CviPII using short endogenous CCD nickase recognition sites.

Amplified material is detected by direct capture on a lateral flow strip (capture sequence 4×TGTA for Ng and 4×TTGA for Ct). Cap-SDA reactions contained 50 mM Tris pH 8.75, 50 mM KOAc, 200 ng/uL T4gp32, 0.5 mM dNTPs, 2.5 mM DTT, 250 nM each of blocked SDA primers ((having a length of 29 nucleotides or 32 nucleotides and blocked with 3C6)), 0.5 U/uL Bsu DNAP, 0.05 U/uL Nt.CviPII Nickase from high concentration stocks (165 U/uL Bsu, 20 U/uL Nt.CviPII), 250 nM of amplicon capture probes. The target dsDNA was diluted in 30 mM MgOAc, then added to the reaction to initiate it (final MgOAc was 15 mM). Reaction was run at room temperature for 30 minutes before running on the lateral flow strip. Running buffer for the lateral flow contained 50 mM Tris pH 8.75, 50 mM KOAc, 2% Ecosurf, and 2 uM of a Poly-T oligo to reduce background.

Nt.CviPII workflow using a lateral flow readout was multiplex capability (FIG. 32). Specifically, two primer pairs were independently amplify in each other's presence, and their products were independently detected on a lateral flow strip using different pulldown capture sequences (FIG. 32).

### Example 26: Amplification Using a Nickase Nt.CviPII

This example demonstrates detection of a dsDNA genomic target (Ng extracted genome) with a Cap-SDA reaction with the nickase Nt.CviPII using short endogenous CCD nickase recognition sites.

Amplified material is detected by direct capture on a lateral flow strip (capture sequence 4×TGTA). The “Biophos” capture probe used is either single-stranded or contains a hairpin. Cap-SDA reactions contained 50 mM Tris pH 8.75, 50 mM KOAc, 500 ng/uL T4gp32, 0.5 mM dNTPs, 2.5 mM DTT, 250 nM each of blocked SDA primers (having both having a length of 29 nucleotides and blocked with 3C6), 0.5 U/uL Bsu DNAP, 0.05 U/uL Nt.CviPII Nickase from high concentration stocks (165 U/uL Bsu, 20 U/uL Nt.CviPII), 250 nM of amplicon capture probes. The target dsDNA was diluted in 30 mM MgOAc and in the presence of 10 ng/uL background hgDNA, then added to the reaction to initiate it (final MgOAc was 15 mM). Reaction was run at room temperature for 30 minutes before running on the lateral flow strip. Running buffer for the lateral flow contained 50 mM Tris pH 8.75, 50 mM KOAc, 2% Ecosurf, and 2 uM of a Poly-T oligo to reduce background.

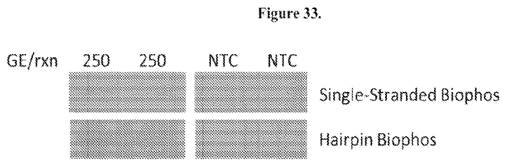

Background DNA is known to be the main inhibitory compound found in clinical sample matrices. The present Example shows enhanced performance of cSDA assays in the presence of human genomic DNA using a hairpin stabilizer-Biophos SDA primer (FIG. 33).

### Example 27: Amplification Using Single Stranded or a Double Stranded Hairpin SDA Primer

This example demonstrates detection of a synthetic ssDNA target (TGAGGAGGAGGAAGTGATGACGAGTGTCTACTCCTCCTCA (SEQ ID NO: 7)) with a Cap-SDA reaction with primer stabilizers that are either single stranded or a double stranded hairpin.

Cap-SDA reactions contained 50 mM Tris pH 8.75, 50 mM KOAc, 100 ng/uL or 200 ng/uL of T4gp32 as indicated, 0.5 mM dNTPs, 0.2× Evagreen DNA dye, 2.5 mM DTT, 1 uM of a blocked SDA primer (GAAGGTCGAAGATCGCTGAGGAGGAG (SEQ ID NO: 8) blocked with 3SpC3 and a SDA primer having a length of 38 nucleotides and blocked with 3SpC3), 0.5 U/uL Bsu DNAP, 1 U/uL Nb.BbvCI Nickase from high concentration stocks (165 U/uL Bsu, 400 U/uL Nb.BbvCI. The target ssDNA was diluted in 75 mM MgOAc, then added to the reaction to initiate it (final MgOAc was 15 mM). Reaction was run at room temperature. Average of two replicates shown.

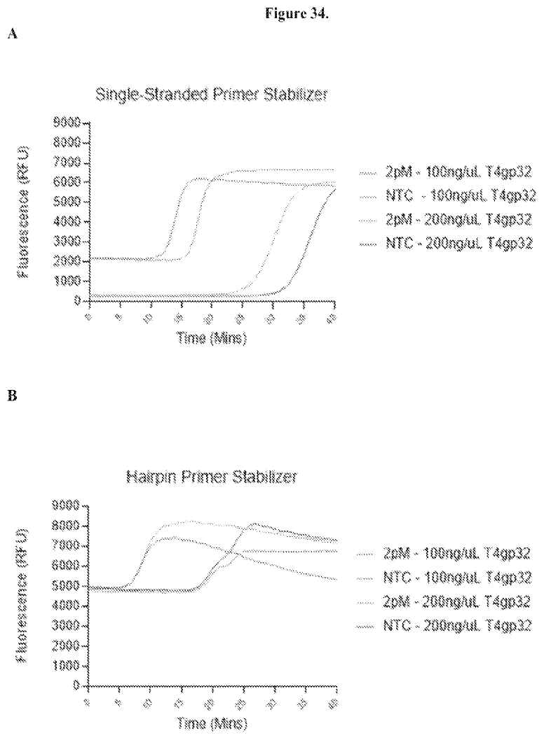

The results show that amplification using a SDA primer having a hairpin stabilizer results in faster amplification compared to a SDA primer having a single-stranded stabilizer, i.e., the detection time is reduced when a SDA primer having a hairpin stabilizer is used (FIG. 34). Furthermore, the performance of hairpin stabilizers are independent of T4gp32 concentration, whereas single-stranded stabilizers suffer dramatically at high T4gp32 concentrations (FIG. 34). Using a hairpin stabilizer allows for a higher T4gp32 concentration, which is highly beneficial in the presence of clinical sample matrices with significant amounts of background DNA.

### Example 28: Amplification Using Single Stranded or a Double Stranded Hairpin SDA Primer

This example demonstrates detection of a dsDNA genomic target (Ng extracted genome) with a Cap-SDA reaction with primer stabilizers that are either single stranded or a double stranded hairpin. The reaction was performed in the presence or absence of 20 ng of human genomic DNA background.

Cap-SDA reactions contained 50 mM Tris pH 8.75, 50 mM KOAc, 200 ng/uL T4gp32, 0.5 mM dNTPs, 1 mM rNTPs, 2.5 mM DTT, 5 U/uL T7 RNAP, 250 nM each of blocked SDA primers (SEQ ID NO: 3 blocked with 3C6, SEQ ID NO: 4 blocked with 3C6, or SDA primers having a length of 36 nucleotide and blocked with 3C6), 200 nM of blocked T7-SDA primer (TAATACGACTCACTATAGGGCCGAGAAGTG (SEQ ID NO: 9) blocked with 3SpC3), 0.5 U/uL Bsu DNAP, 0.05 U/uL Nt.CviPII Nickase from high concentration stocks (165 U/uL Bsu, 20 U/uL Nt.CviPII), 200 nM of FQ reporter (SEQ ID NO: 2 comprising a 56FAM fluorophore and a 3BQH1 quencher), and 10 nM of Cas13/crRNA complex (GAUUUAGACUACCCCAAAAACGAAGGGGACUAAAACAGACACUCGUCAUCAC UUCU (SEQ ID NO: 10)). The target ssDNA was diluted in 75 mM MgOAc, then added to the reaction to initiate it (final MgOAc was 15 mM). Reaction was run at room temperature. Average of two replicates shown.

FIG. 35 shows that amplification using a Cap-SDA Primer having a single-stranded stabilizer was not capable of stable detection of a target nucleic acid with human genomic DNA present, whereas a Cap-SDA primer having a hairpin stabilizer showed stable detection of a target nucleic acid in the presence of human genomic DNA compared to amplification and detection without the presence of human genomic DNA (FIG. 35). Hairpin stabilizer can help recover loss of signal when amplifying in the presence of human background DNA. Using Cap-SDA primers having a hairpin stabilizer instead of Cap-SDA primers having a single-stranded stabilizer tolerates high T4gp32 concentrations.

### Example 29: SDA Using an Nt.CviPII Nickase

This example demonstrates detection of a synthetic ssDNA target (CCATGCACGTGCGAAGAAGCTATAAGACATGTACGTGCATGGACTTCTAGCTGC AGCA (SEQ ID NO: 11)) with a Cap-SDA reaction with an Nt.CviPII nickase, using short CCD nickase recognition sites.

Cap-SDA reactions contained 50 mM Tris pH 8.75, 50 mM KOAc, 400 ng/uL T4gp32, 0.5 mM dNTPs, 2.5 mM DTT, 500 nM of blocked SDA primer (TGCTGCAGCTAGAAGTCCATGCACGT (SEQ ID NO: 12) blocked with 3C6), 0.2× Evagreen DNA dye, 0.5 U/uL Bsu DNAP, 0.05 U/uL Nt.CviPII Nickase from high concentration stocks (165 U/uL Bsu, 20 U/uL Nt.CviPII). The target ssDNA was diluted in 30 mM MgOAc, then added to the reaction to initiate it (final MgOAc was 15 mM). Reaction was run at room temperature. The SDA Primer was either pre-treated or not with Klenow Large Fragment (KF) by incubating in 50 mM Tris pH 8.75, 50 mM KOAc, 10 mM MgOAc, and 1 U/uL or none of Klenow Large Fragment, for 1 hour at 37 C followed by inactivation at 85 C for 20 min.

Klenow Fragment DNAP having specific 3′ exo activity was used to improve the purity of the Cap-SDA primer pool and enrich for SDA primers that have blocked 3′ ends. Cap-SDA primers having a free 3′ end can be extended by the DNA polymerase.

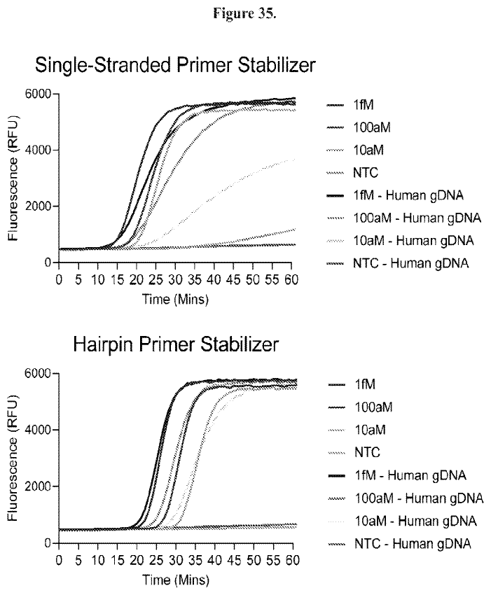

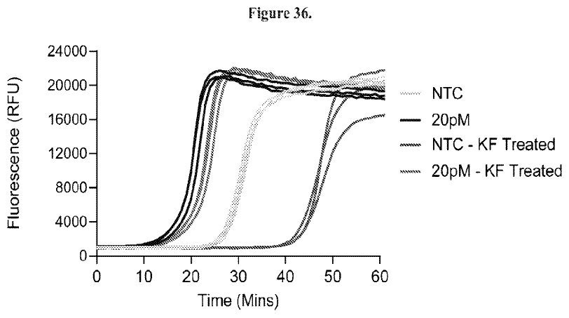

This data shows that removing uncapped primers that have free 3′ ends reduce the rate of primer dimer formation, thereby improving assay performance (FIG. 36).

### Example 30: Amplification Using a Nickase Nt.CviPII

This example demonstrates detection of synthetic ssDNA targets (SEQ ID NOs: 31, 32, 33, 34 shown in Table 3) with a Cap-SDA reaction with the nickase Nt.CviPII, using short CCD nickase recognition sites.

TABLE 3

| | Synthetic ssDNA targets |
|---|---|
| SEQ ID | TAGAAGTCCGAAACAAAATAATTTAAGTTTAA |
| NO: 13 | TGTTTTCAAAAAATTATAAAAGTCTTGG |
| SEQ ID | TAGAAGTCCAGAAAATCACAAGTTCATTATTA |
| NO: 14 | CTCAAAAAGAAGCACTTGCTAACTATATGCGG |
| SEQ ID | TAGAAGTCCGAAACAAAATAATTTAAGTTTAA |
| NO: 15 | TGTTTCCAAAAAATTATAAAAGTCTTGG |
| SEQ ID | TAGAAGTCCAGAAAATCACAAGTTCATTATTA |
| NO: 16 | CTCCAAAAGAAGCACTTGCTAACTATATGCGG |

Cap-SDA reactions contained 50 mM Tris pH 8.75, 50 mM KOAc, 400 ng/uL T4gp32, 0.5 mM dNTPs, 2.5 mM DTT, 500 nM of blocked SDA primer (blocked with 3C6), 0.2× Evagreen DNA dye, 0.5 U/uL Bsu DNAP, 0.05 U/uL Nt.CviPII Nickase from high concentration stocks (165 U/uL Bsu, 20 U/uL Nt.CviPII). The target ssDNA was diluted in 30 mM MgOAc, then added to the reaction to initiate it (final MgOAc was 15 mM). Reaction was run at room temperature. The ssDNA targets either contained no internal nicking sequences in the amplicon region, or one internal nicking sequence in the amplicon region.

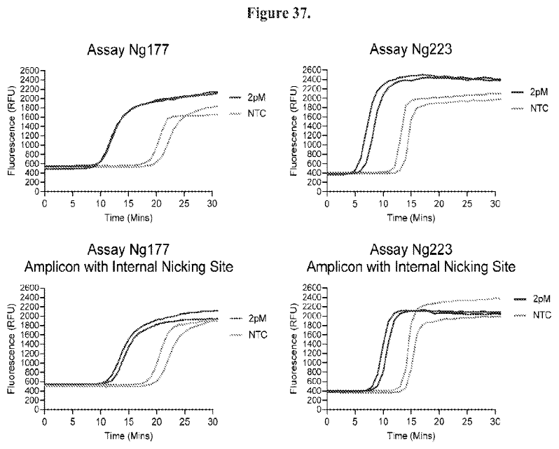

This data demonstrates that when using the Nt.CviPII workflow targeting two opposing native nick sites, regions of interest can be amplified even if there are additional endogenous nick sites present internal within the amplicon (ie, between the two opposing nick sites that the primers are targeting) (FIG. 37). This allows for a much broader range of sequences to be targeted, giving more flexibility in designing around conserved sequences and unwanted similarity to crossreactive species.

### Example 31: Sponge Oligo Nucleotides Neutralize T4gp32 Related Background

This example demonstrates that addition of Sponge Oligo nucleotides to the running buffer can remove background testline signal.

Capped SDA reaction contained 50 mM Tris pH 8.75, 50 mM KOAc, 200 ng/uL T4gp32, 0.5 mM dNTPs, 2.5 mM DTT, and 250 nM of amplicon capture probes were diluted 1:1 with running buffer containing either 50 mM Tris pH 8.75 and 50 mM KOAc, 10% Skim milk in 50 mM Tris pH 8.75 and 50 mM KOAc, or 2 uM of a “Sponge Oligo” in 50 mM Tris pH 8.75 and 50 mM KOAc. The mixes were then run on lateral flow strips designed to pull down a positive control oligo either by hapten pulldown or oligo pulldown.

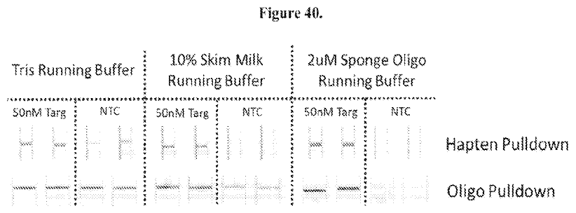

When reactions contain both T4gp32 and capture probes are run on lateral flow strips, such as Hapten Pulldown or Oligo Pulldown, using only Tris Running Buffer significant background testline signal is observed (See FIG. 40). Background noise is induced by the presence of T4gp32 combined with detection probes. Addition of milk protein to the running buffer, as a blocking method, reduced some background testline signal (FIG. 40). Addition of a Sponge Oligo in the running buffer was efficient method to neutralize the background (FIG. 40). Sponge Oligos presumably bind to the excess T4gp32 and neutralizes its background inducing behavior.

### Example 32: Ambient Temperature Viral Particle Lysis

This example demonstrates effective viral particle lysis and effective inhibition of RNase at ambient temperature, through use of lysis technologies provided herein.

### Viral Lysis

Frozen titered viral stocks of FluA and SARS-CoV-2 were added to a 10 mM Tris-HCl, 1 mM EDTA (pH 8.0) buffer (TE buffer) or one of 3 nasal swab matrices (created by eluting one anterior nasal swab in 1 mL of TE buffer). These contrived specimens were treated with 20 mM Sodium hydroxide (NaOH) or a 95° C./3 minute heat lysis step with 5 mM TCEP present (a reducing agent) was used as a positive control. A portion of the lysed material was added to a room temperature (22° C.) reverse transcriptase (RT) reaction, to convert released viral RNA to cDNA in a 1:1 ratio. The RT reaction was stopped by heat inactivating the RT reaction. A portion of the RT reaction was added to a standard qPCR reaction with primers and tagman probes specific for the appropriate virus.

Viral particle lysis preparations were assessed by measuring Cq value of the samples. Poor to no viral particle lysis was indicated by high Cq values, while optimum viral particle lysis was indicated by lower Cp values.

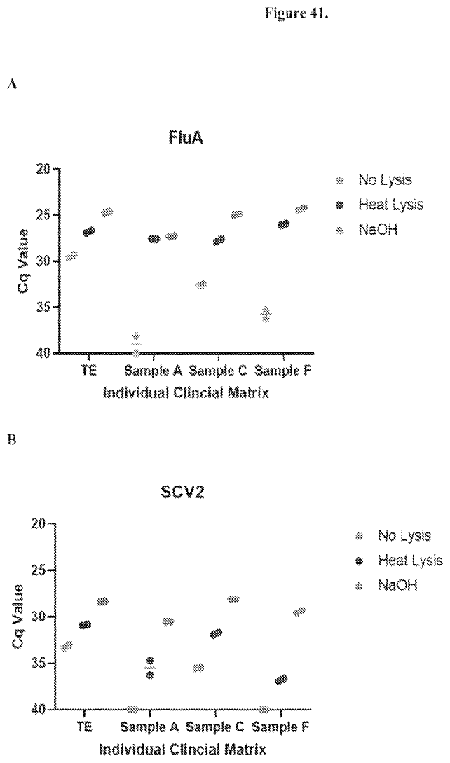

The results show that FluA (FIG. 41A) and SARS-CoV-2 (SCV2) (FIG. 41B) virus can be effectively lysed in and detected from nasal swab matrix with 20 mM NaOH at room temperature comparable to or better than heat lysis control condition.

### RNase Inhibition

A nasal swab matrix was created by eluting one anterior nasal swab in 1 mL of TE buffer. The nasal swan matrix was subsequent treated with a commercially available RNase inhibitor or 15 mM NaOH. Remaining RNase activity was analyzed using a standard RNase Alert protocol.

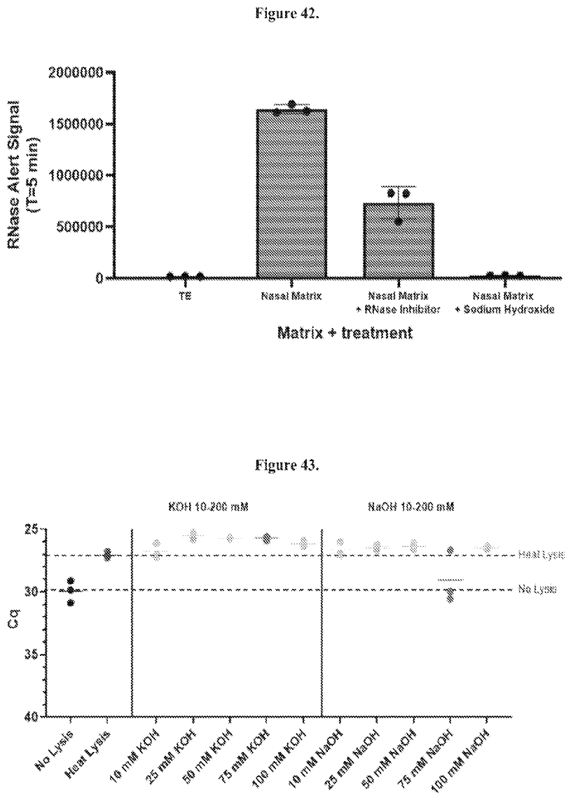

The results show that NaOH treatment effectively reduced the amount of RNase activity present in the sample (FIG. 42). NaOH has the additional benefit of inhibiting RNases present in the clinical matrix, protecting the released viral genomes, both from pre-lysed virions as well as virions lysed by the treatments.

### pH Solutions

Samples were generated by diluting a SARS-CoV-2 (SCV2) viral stock with TE buffer. The samples were treated with KOH concentrations of 10 mM, 25 mM, 50 mM, 75 mM or 100 mM, or NaOH concentrations of 10 mM, 25 mM, 50 mM, 75 mM or 100 mM. 95° C./3 minute heat lysis step was used as a positive lysis control. A portion of the lysed material was added to a room temperature (in this case 22° C.) reverse transcriptase (RT) reaction, to convert released viral RNA to cDNA in a 1:1 ratio. The RT reaction was stopped by heat inactivating the RT. A portion of the RT reaction was then added to a standard qPCR reaction with primers and tagman probes specific for the appropriate virus.

Viral particle lysis preparations were assessed by measuring Cq value of the samples. Poor to no viral particle lysis was indicated by high Cq values, while optimum viral particle lysis was indicated by lower Cp values.

The results show that both NaOH and KOH are effective at releasing viral nucleic acids over a range of concentrations (FIG. 43).

### Hydroxide-Based Chemical Lysis of Non-Enveloped Virus

Samples were prepared by diluting a human adenoviral stock in TE buffer. Samples were left at room temp (22° C.), heated t 95° C. (heat lysis), or treated with increasing concentrations (20 mM, 40 mM, 60 mM, 80 mM, 100 mM, or 200 mM) of NaOH for 5 minutes.

After treatment, the solutions were analyzed by qPCR to determine viral nucleic acid release using human adenoviral specific PCR primers and a taqman probe

The results show that NaOH lysis at room temperature of non-enveloped human adenoviral releases more DNA than heat lysis (FIG. 44).

### Example 33: Ambient Temperature Bacterial Lysis

This example demonstrates effective bacterial lysis at ambient temperature through use of lysis technologies provided herein.

### Bacteria Lysis

Samples were generated by diluting freshly grown N. gonorrhoeae or C. Trachomatis (from frozen stock) with TE buffer. Samples were hereafter treated with KOH concentrations of 10 mM, 25 mM, 50 mM, 75 mM or 100 mM. Bead beating (bead lysis) was used as a positive control. A portion of the lysate was then added to a standard qPCR reaction with primers and tagman probes specific for the appropriate bacterium.

Bacterial lysis preparations were assessed by measuring Cq value of the samples. Poor to no bacterial lysis was indicated by high Cq values, while optimum viral particle lysis was indicated by lower Cq values.

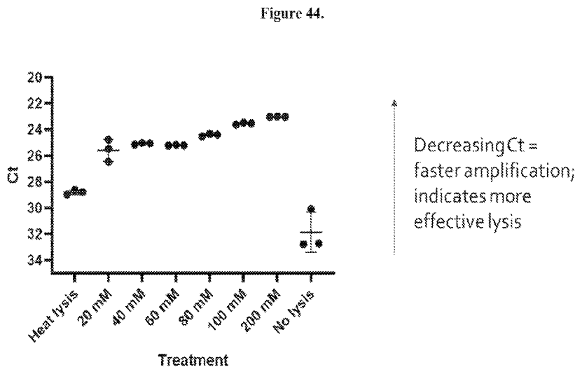

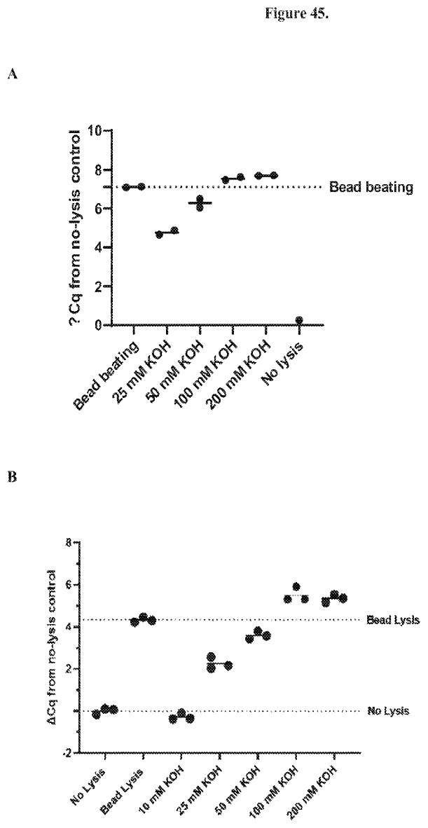

The results show that KOH lysis with high KOH concentrations, specifically above 25 mM, are efficient at releasing bacterial nucleic acids over a range of concentrations (FIG. 45A-B). In particular, concentrations at 100 mM and 200 mM showed similar or better lysis than bead lysis (FIG. 45A-B).

### Addition of Detergents

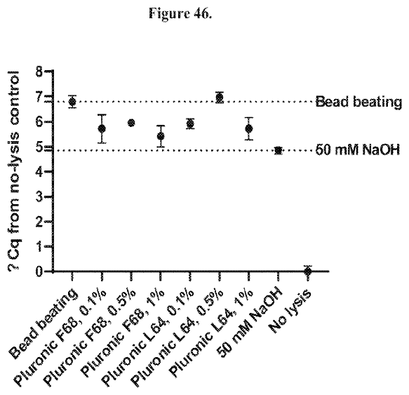

Samples were generated by diluting N. gonorrhoeae with TE buffer. Samples were hereafter treated with 50 mM KOH and an additional detergent as shown in FIG. 46. Bead beating (bead lysis) was used as a positive control.

Addition of a detergent allows for higher lysis efficiency at lower concentrations of KOH.

### Example 34: Lysis at Varying Incubation Temperatures

This example demonstrates effective bacterial lysis at varying temperatures through use of lysis technologies provided herein.

Samples were prepared by diluting N. gonorrhoeae in TE buffer. Samples were treated with KOH or additives as indicated below and incubates for 3-5 minutes at indicated temperatures. 95° C. heat lysis was used as a positive control. A portion of the lysates were added to a PCR reaction and the nucleic acid concentration, reflecting the nucleic acid release, was measured for each sample. Results are normalized to heat lysis (95° C.) at 100%.

### PI64 9

Samples were treated with KOH or HCl or PI64 9 0.5% and incubated at room temperature (22° C.), 50° C. or 65° C., see the table below.

TABLE 4

| Chemical | Temp & incubation time | Detergent & conc |
|---|---|---|
| KOH 50 mM | Rm Temp (22° C.) | |
| 13.5 mM HCL | 50° C., 3 min | Pl64 9 (0.5%) |
| TE (pH 8) | 75° C., 3 min | |
| | 95° C., 5 min | |

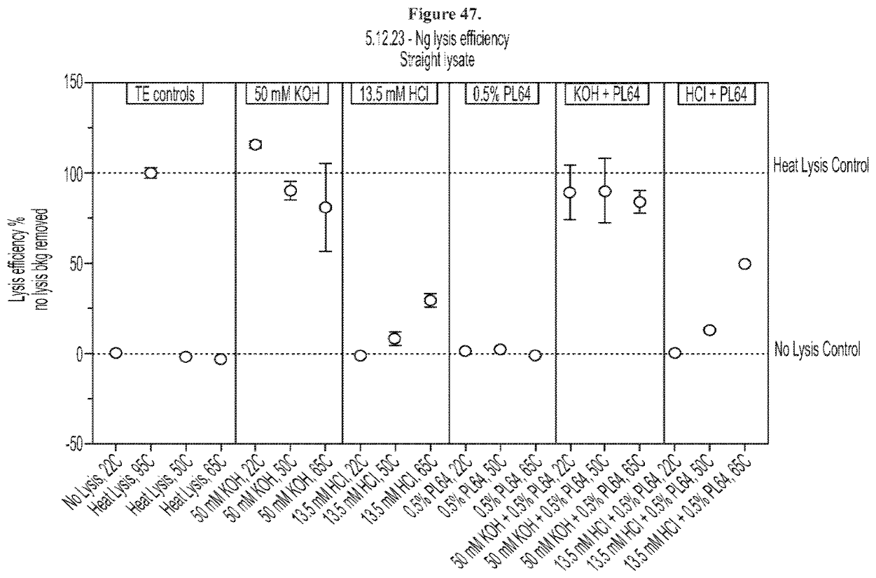

The results show that P164 alone is ineffective and that 50 mM KOH achieve effective bacteria lysis, regardless of the temperature or addition of an additional detergent (FIG. 47). Treatment with 13.5 mM HCl demonstrated better lysis at higher temperatures and with the addition of an additional detergent, but not as well as KOH (FIG. 47).

### ESH9

Samples were treated with KOH or HCl and 3% ESH9 and incubated for 3-5 minutes at room temperature (22° C.), 50° C. or 70° C.

TABLE 5

| Chemical | Temp & incubation time | Detergent & conc |
|---|---|---|
| KOH 50 mM | Rm Temp (22° C.) | |
| 13.5 mM HCL | 50° C., 3 min | ESH9 (3%) |
| TE (pH 8) | 70° C., 3 min | |
| | 95° C., 5 min | |

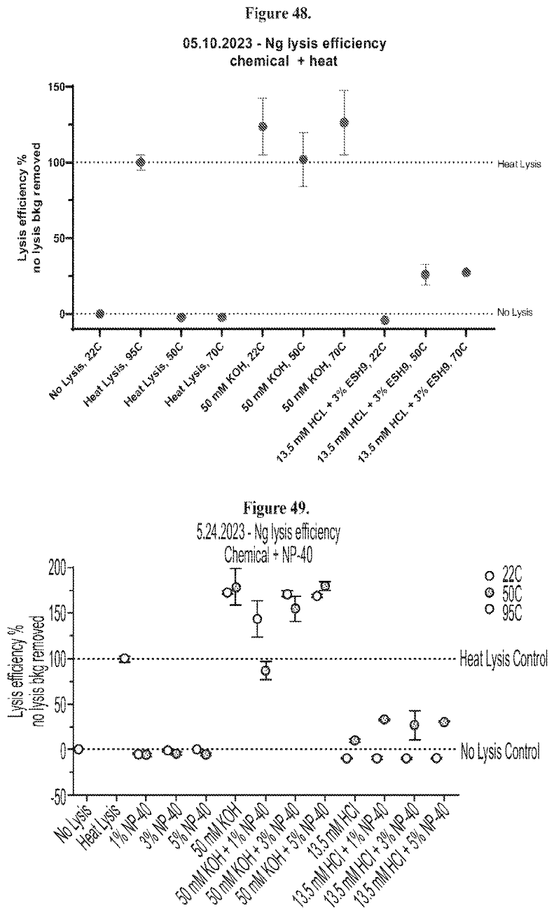

The results show effective N. gonorrhoeae lysis using 50 mM KOH regardless of lysis temperature. Treatment with HCl+Ecosurf demonstrated better lysis a higher temperatures, specifically at 50° C. and 65° C. lysis, but less effective than KOH (FIG. 48). Additionally, no lysis was observed at 50° C. or 70° C. incubation alone, without any detergents or additives (FIG. 48).

### NP40—Constant KOH Concentration

Samples were treated with 50 mM KOH or HCl and 1%-3% NP40 and incubated for 3-5 minutes at room temperature (22° C.) or 50° C.

TABLE 6

| Chemical | Temp & incubation time | Detergent & conc |
|---|---|---|
| KOH 50 mM | Rm Temp (22° C.) | |
| 13.5 mM HCL | 50° C., 3 min | NP40 (1%-3%) |
| TE (pH 8) | | |
| | 95° C., 5 min | |

The results show that NP40 alone is ineffective for gDNA release and that 50 mM KOH achieve effective bacteria lysis, regardless of the temperature or addition of an additional detergent (FIG. 49). Treatment with 13.5 mM HCl demonstrated better lysis at higher temperatures and with the addition of an additional detergent, but not as well as KOH (FIG. 49).

### NP40—Various KOH Concentrations

Samples were treated with 0 mM to 50 mM KOH or HCl and 3% NP40 and incubated for 3-5 minutes at room temperature (22° C.) or 50° C.

TABLE 7

| Chemical | Temp & incubation time | Detergent & conc |
|---|---|---|
| KOH 0 mM-50 mM | Rm Temp (22° C.) | |
| | 50° C., 3 min | NP40 (3%) |
| TE (pH 8) | | |
| | 95° C., 5 min | |

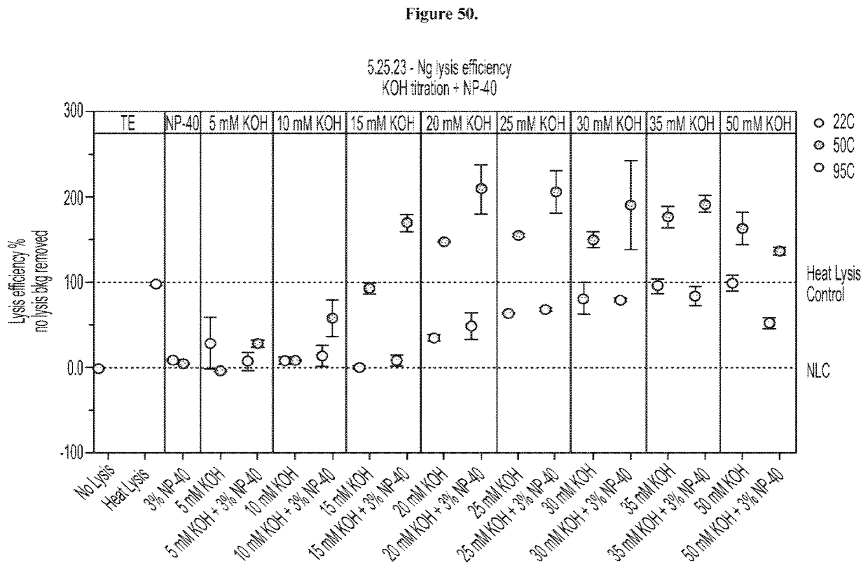

The results show that in the presence of 3% NP40 lower concentrations of KOH can be used to achieve the same lysis as 50 mM KOH alone (FIG. 50).

### Improved LAMP-Cas Detection

Samples were treated with 50 mM KOH and incubated for 3-10 minutes at room temperature (22° C.). Lysed samples comprising released nucleic acids were amplified by LAMP and detected by a Cas detection system.

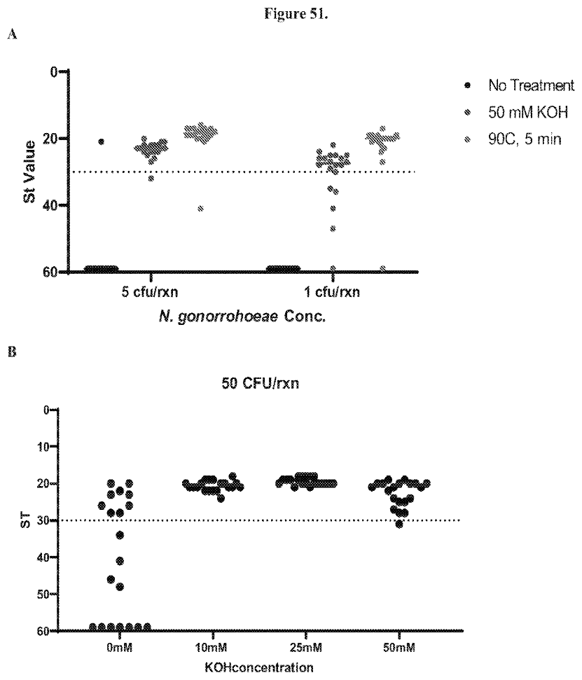

The results show an improvement in LoD when samples were lysed with either KOH or heat lysis compared to no lysis control (FIG. 51).

### Example 35: KOH Lysis of N. gonorrhoeae in Vaginal Matrix in LAMP-Cas

This example demonstrates effective bacterial lysis in a vaginal matrix through use of lysis technologies provided herein.

Samples were prepared by diluting N. gonorrhoeae in vaginal swab matrix (1 swab eluted in 3 mL buffer). Samples were treated with 0 mM, 15 mM, 25 mM, 50 mM, or 85 mM KOH and DNA detected by LAMP-Cas reactions (run at 60 C in ABIQS5).

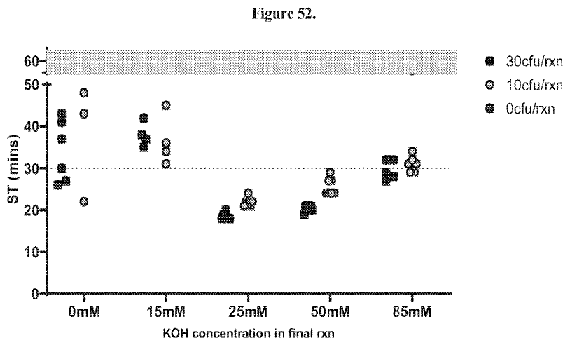

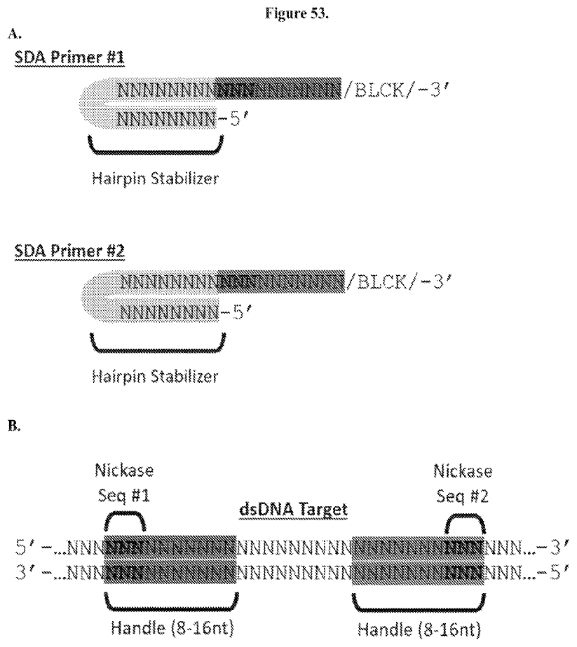

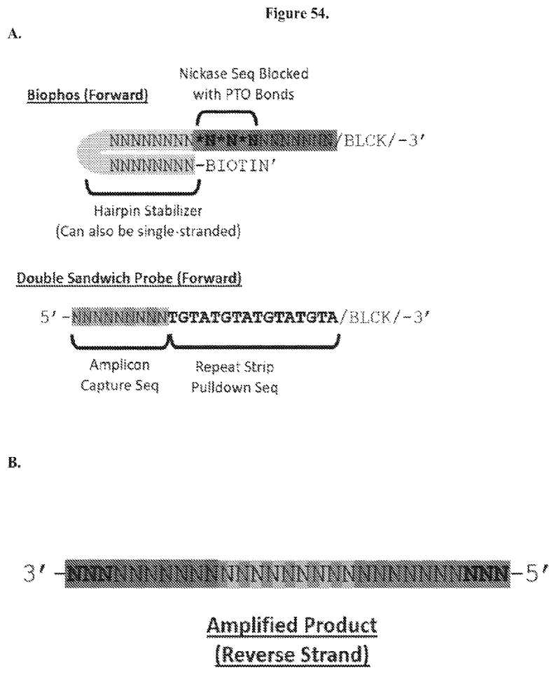

The results show that a low KOH concentration, specifically 15 mM KOH, do not improve the sensitivity for the LAMP Cas reaction over no KOH treatment. When the bacterial cells in vaginal matrix are treated with higher concentrations of KOH, specifically 25 mM or higher, the sensitivity of the LAMP Cas reactions are improved (FIG. 52). Treatment of the bacterial cells with >25 mM KOH results in more released genomic DNA for downstream amplifications.

### EQUIVALENTS

Those skilled in the art will recognize, or be able to ascertain using no more than routine experimentation, many equivalents to the specific embodiments of the invention provided herein. The scope of the present invention is not intended to be limited to the above Description, but rather is as set forth in the following claims:

1. A composition comprising:
(a) a double-stranded DNA target nucleic acid sequence comprising a first native restriction enzyme recognition sequence and a second native restriction enzyme recognition sequence on opposite strands separated by a target nucleic acid region;
(b) a first strand displacement amplification (SDA) primer comprising:
(i) a sequence complementary to the first native restriction enzyme recognition sequence;
(ii) a 3′ blocking molecule; and
(iii) a 5′ stabilization sequence comprising at least 8 nucleotides;
(c) a second SDA primer comprising:
(i) a sequence complementary to the second restriction enzyme nickase recognition sequence;
(ii) a 3′ blocking molecule; and
(iii) a 5′ stabilization sequence comprising at least 8 nucleotides;
(d) a cleavage enzyme;
(e) a DNA polymerase having strand displacement activity;
(f) a single stranded binding protein; and
(g) deoxynucleotide triphosphates (dNTPs).

2. The composition of claim 1, wherein the 3′ blocking molecule of the first and/or second SDA primer is selected from the group consisting of 3′ ddNTP, 3′ Inverted dT, a 3′ carbon chain spacer, a 3′ hexanediol, a 3′ amino spacer, and a 3′ phosphorylation, wherein the 3′ carbon chain spacer is a carbon chain bound to the 3′ OH group blocking elongation.

3. The composition according to claim 1, wherein at least one of the stabilization sequences is a hairpin stabilization sequence.

4. (canceled)

5. The composition according to claim 1, wherein at least one of the 5′ stabilization sequences comprises a T7 promoter sequence.

6. The composition according to claim 1, wherein:
(i) the DNA polymerase is selected from the group consisting of Bsu DNAP, Klenow LF, Klenow Exo-, and Isopol, and Bst DNAP;
(ii) the single stranded binding protein is a T4gp32, or
(iii) the concentration of the single stranded binding protein is at least 200 ng/μl.

7-8. (canceled)

9. The composition according to claim 1, wherein the composition further comprises KOAc or PEG.

10. (canceled)

11. The composition according to claim 1, wherein the first and second native restriction enzyme recognition sequences are;
(i) about 3 to about 7 nucleotides;
(ii) native nickase recognition sequences; or
(iii) CCD nickase recognition sequences, wherein the nickase is an Nt.CviPII nickase.

12-15. (canceled)

16. The composition according to claim 1, wherein at least one of the dNTPs is a modified dNTP.

17. The composition according to claim 1, wherein the first SDA primer sequence complementary to the first native restriction enzyme recognition sequence and the second SDA primer sequence complementary to the second native restriction enzyme recognition sequence comprise:
(i) one or more modified nucleotides; and
(ii) one or more phosphorothioate (PTO) linkages.

18-22. (canceled)

23. The composition according to claim 1, wherein the composition further comprises:
(h) a detection system comprising:
(i) a guide nucleic acid comprising a nucleic acid sequence complementary to the target nucleic acid region or portion thereof;
(ii) a Cas enzyme with collateral cleavage activity, wherein the Cas enzyme is a Cas13 enzyme or Cas12 enzyme; and
(iii) a nucleic acid reporter probe susceptible to the collateral cleavage activity, so that the nucleic acid reporter probe has a detectably different first uncleaved state and a second cleaved state.

24-25. (canceled)

26. The composition according to claim 23, wherein the nucleic acid reporter probe comprises a fluorophore and a quencher.

27. The composition according to claim 1, wherein the composition further comprises:
(h) a detection system comprising:
(i) a capture probe; and
(ii) a conjugate capture probe comprising a detectable label and a 3′ blocking entity.

28. The composition according to claim 27, wherein:
(i) the capture probe is a biotinylated capture probe; and
(ii) wherein the conjugate capture probe is about 10 to about 20 nucleotides.

29. The composition according to claim 27, wherein the capture probe comprises a stabilization sequence.

30. (canceled)

31. A method comprising:
(a) amplifying a double-stranded DNA target nucleic acid sequence comprising a first native restriction enzyme recognition sequence and a second native restriction enzyme recognition sequence on opposite strands separated by a target nucleic acid region in a sample, wherein amplifying comprises:
(A) contacting the sample with a composition comprising:
(a) a cleavage enzyme;
(b) a DNA polymerase having strand displacement activity;
(c) a single stranded binding protein; and
(d) dNTPs;
thereby generating a single stranded DNA (ssDNA) cassette;
(B) contacting the ssDNA cassette generated in step (A) with a composition comprising:
(e) a first SDA primer comprising:
(i) a sequence complementary to the first native restriction enzyme recognition sequence;
(ii) a 3′ blocking molecule; and
(iii) a 5′ stabilization sequence comprising at least nucleotides;
(f) a second SDA primer comprising:
(i) a sequence complementary to the second native restriction enzyme recognition sequence;
(ii) a 3′ blocking molecule; and
(iii) a 5′ stabilization sequence comprising of at least 8;
(g) the cleavage enzyme;
(h) the DNA polymerase having strand displacement activity;
(i) the single stranded binding protein;
(j) the dNTPs; and
thereby generating a reaction mixture; and
(C) incubating the reaction mixture under conditions favorable for generation of multiple copies of nucleic acid identical or complementary to the ssDNA cassette,
(b) detecting the double-stranded DNA target nucleic acid sequence, wherein the detecting comprises:
(1)
(A) contacting at least one of the multiple copies of an amplified target nucleic acid, with a composition comprising:
(i) a guide nucleic acid comprising a nucleic acid sequence complementary to the DNA cassette; and
(ii) a Cas enzyme with collateral cleavage activity:
(iii) a nucleic acid reporter probe susceptible to the collateral cleavage activity, so that the nucleic acid reporter probe has a detectably different first uncleaved state and a second cleaved state; and
(B) detecting cleavage of the nucleic acid reporter probe by detecting a difference between the first state and the second state to determine the presence of the target nucleic acid sequence in the sample: or
(2)
(A) contacting at least one of the multiple copies of the amplified target nucleic acid, with a composition comprising
(i) a capture probe; and
(ii) a conjugate capture probe comprising a detectable label and a 3′ blocking entity; and
(B) detecting presence of the detectable label bound to a solid substrate to determine the presence of the target nucleic acid sequence in the sample,
wherein the amplifying and the detecting are performed (i) at ambient temperature; (ii) without temperature cycling; and (iii) at most at 50° C.

32-80. (canceled)

81. The composition according to claim 1, wherein the at least one nickase recognition sequences is a GCTGAGG nickase recognition sequences; and wherein the nickase is an Nb.BbvCl nickase.

82-138. (canceled)

139. A composition comprising:
(a) a RNA target nucleic acid;
(b) a reverse primer comprising:
(i) a 3′ sequence complementary to the RNA target nucleic acid; and
(ii) a 5′ SDA primer binding sequence, wherein the primer binding sequence comprises a restriction enzyme recognition sequence, a partial restriction enzyme recognition sequence, or complements thereof,
(c) a reverse transcriptase, wherein the reverse transcriptase has RNASEh activity, and is selected from the group consisting of MMLV, AMV, Protoscript II, Superscript I and II and II and IV, RTx, GOScript, Sensiscript, Primescript, and Maxima; and
(d) a forward primer comprising:
(i) a 3′ sequence complementary to the RNA target polynucleotide; and
(ii) a 5′ SDA primer binding, wherein the primer binding sequence comprises a restriction enzyme recognition sequence or a partial restriction enzyme recognition sequence, or complements thereof,
(e) a first SDA primer comprising:
(i) a sequence complementary to the 5′ SDA primer binding sequence of the reverse primer;
(ii) a 3′ blocking molecule; and
(iii) a 5′ stabilization sequence comprising at least 8 nucleotides, and when the reverse primer comprises only a partial restriction enzyme recognition sequence the first SDA primer comprises a partial restriction enzyme recognition sequence that together with the partial restriction enzyme recognition sequence in the 5′ SDA primer binding sequence of the reverse primer form a complete restriction enzyme recognition sequence;
(f) a second SDA primer comprising:
(i) a sequence complementary to the 5′ SDA primer binding sequence of the forward primer;
(ii) a 3′ blocking molecule; and
(iii) a 5′ stabilization sequence comprising at least 8, wherein the forward primer comprises only a partial restriction enzyme recognition sequence the first SDA primer comprises a partial restriction enzyme recognition sequence that together with the partial restriction enzyme recognition sequence in the 5′ SDA primer binding sequence of the forward primer form a complete restriction enzyme recognition sequence;
(g) a cleavage enzyme;
(h) a polymerase having strand displacement activity;
(i) a single stranded binding protein; and
(j) dNTPs;
(k) KOAc or PEG, and
(l) a detection system comprising:
(A)
(i) a guide nucleic acid comprising a nucleic acid sequence complementary to the target nucleic acid region or portion thereof; and
(ii) a Cas enzyme with collateral cleavage activity;
(iii) a nucleic acid reporter probe susceptible to the collateral cleavage activity, so that the nucleic acid reporter probe has a detectably different first uncleaved state and a second cleaved state: or
(B)
(i) a capture probe; and
(ii) a conjugate capture probe comprising a detectable label and a 3′ blocking entity
wherein the restriction enzyme recognition sequence in (b), (d), (e), or (f) is a nickase recognition sequence, wherein the nickase is an Nt.CviPII nickase or an Nb.BbvCI nickase.

140-141. (canceled)

142. The composition according to claim 139, wherein the composition further comprises a bump primer.

143-167. (canceled)

168. A method comprising:
(1) amplifying a RNA target nucleic acid sequence in a sample comprising:
(A) contacting the sample, with a composition comprising
(a) a reverse primer comprising:
(i) a 3′ sequence complementary the RNA target nucleic acid; and
(ii) a 5′ SDA primer binding sequence, wherein the primer binding sequence comprises a restriction enzyme recognition sequence, a partial restriction enzyme recognition sequence, or complements thereof,
(b) a reverse transcriptase; and
(c) a forward primer comprising:
(i) a 3′ sequence complementary the RNA target nucleic acid; and
(ii) a 5′ SDA primer binding sequence, wherein the primer binding sequence comprises a restriction enzyme recognition sequence, a partial restriction enzyme recognition sequence, or complements thereof,
thereby producing a first reaction mixture;
(B) incubating the first reaction mixture under conditions favorable for generation of a single stranded DNA (ssDNA) cassette;
(C) contacting the ssDNA cassette of (B) with a composition comprising:
(d) a first SDA primer comprising:
(i) a sequence complementary to the 5′ SDA primer binding sequence of the reverse primer;
(ii) a 3′ blocking molecule; and
(iii) a 5′ stabilization sequence comprising at least 8 nucleotides, and when the reverse primer comprises only a partial restriction enzyme recognition sequence the first SDA primer comprises a partial restriction enzyme recognition sequence that together with the partial restriction enzyme recognition sequence in the 5′ SDA primer binding sequence of the reverse primer form a complete restriction enzyme recognition sequence;
(e) a second SDA primer comprising:
(i) a sequence complementary to the 5′ SDA primer binding sequence of the forward primer;
(ii) a 3′ blocking molecule; and
(iii) a 5′ stabilization sequence comprising at least 8 nucleotides, when the forward primer comprises only a partial restriction enzyme recognition sequence the first SDA primer comprises a partial restriction enzyme recognition sequence that together with the partial restriction enzyme recognition sequence in the 5′ SDA primer binding sequence of the forward primer form a complete restriction enzyme recognition sequence; and
(f) at least one cleavage enzyme, polymerase having strand displacement activity, single stranded binding protein, dNTPs; and
thereby producing a second reaction mixture;
(D) incubating the second reaction mixture under conditions favorable for generation of multiple copies of a nucleic acid identical or complementary to the ssDNA cassette, and
(2) detecting the RNA target nucleic acid sequence in a sample comprising, wherein the detecting comprises:
(a)
(A) contacting at least one of the multiple copies of the nucleic acid, with a composition comprising:
(i) a guide nucleic acid comprising a nucleic acid sequence complementary to the DNA cassette; and
(ii) a Cas enzyme with collateral cleavage activity;
(iii) a nucleic acid reporter probe susceptible to the collateral cleavage activity, so that the nucleic acid reporter probe has a detectably different first uncleaved state and a second cleaved state; and
(B) detecting cleavage of the nucleic acid reporter probe by detecting a difference between the first state and the second state to determine the presence of the RNA target nucleic acid sequence in the sample; or
(b)
(A) contacting at least one of the multiple copies of the nucleic acid, with a composition comprising
(i) a capture probe; and
(ii) a conjugate capture probe comprising a detectable label and a 3′ blocking entity; and
(B) detecting presence of the detectable label bound to a solid substrate to determine the presence of the RNA target nucleic acid sequence in the sample.

169-204. (canceled)

205. A composition comprising:
(a) a target polynucleotide;
(b) a first probe comprising a 3′ SDA primer binding sequence comprising a partial nickase recognition site or complement thereof, a nucleic acid sequence that is complementary to the target nucleic acid, and a first nucleic acid sensory part;
(c) a second probe comprising a 5′ SDA primer binding sequence comprising a partial nickase recognition site or complement thereof, a nucleic acid sequence that is complementary to the target nucleic acid, and second nucleic acid sensor part, wherein each nucleic acid sensory part comprises a sequence that is, encodes, or
templates coding of a first and second reporting element component, respectively:
(d) at least one gap filling oligo,
(e) a ligase, and
(f) 2-10% PEG,
when the first and second probe are ligated together, generate a single strand of a DNA cassette that encodes at least one reporter and comprises a 3′ SDA primer binding sequence and a 5′ SDA primer binding sequence, wherein the SDA primer binding sequences comprise a partial nickase recognition sequence or a complement thereof.

206-227. (canceled)

228. A method of detecting a target nucleic acid sequence in a sample comprising:
(a) contacting the sample with a composition comprising
(i) a first probe comprising a 3′ SDA primer binding sequence comprising a partial nickase recognition site or complement thereof, a nucleic acid sequence that is complementary to the target nucleic acid, and a first nucleic acid sensor part;
(ii) a second probe comprising a 5′ SDA primer binding sequence comprising a partial nickase recognition site or complement thereof, a nucleic acid sequence that is complementary to the target nucleic acid, and second nucleic acid sensor part,
wherein each nucleic acid sensory part comprises a sequence that is, encodes, or templates coding of a first and second reporting element component, respectively;
(iii) at least one gap filling oligo, wherein; and
(iv) a ligase,
when ligated together, generate a single strand of a nucleotide cassette that encodes at least one reporter and comprises a 3′ SDA primer binding sequence and a 5′ SDA primer binding sequence, wherein the primer binding sequence comprises a nickase recognition sequence or a partial nickase recognition sequence thereby producing a first reaction mixture;
(b) incubating the first reaction mixture under conditions favorable for generation of a single stranded nucleotide cassette,
(c) contacting the nucleotide cassette of (b) with a composition comprising:
(i) a first SDA primer comprising:
(a) a sequence complementary to the 3′ SDA primer binding sequence;
(b) a 3′ blocking molecule; and
(c) a stabilization sequence comprising at least 8 nucleotides and comprising a partial nickase recognition site that together with the partial nickase recognition site on the 3′ SDA primer binding sequence form a complete nickase primer binding site;
(ii) second SDA primer comprising:
(a) sequence complementary to the 5′ SDA primer binding sequences;
(b) 3′ blocking molecule; and
(c) a 5′ stabilization sequence comprising at least 8 nucleotides, comprising a partial nickase recognition site that together with the partial nickase recognition sequence in the 5′ SDA primer binding sequence form a complete nickase primer binding sequence; and
(iii) at least one nickase, polymerase having strand displacement activity, and single stranded binding protein;
thereby producing a second reaction mixture; and
(d) incubating the second reaction mixture under conditions favorable for generation of multiple copies of a nucleic acid identical or complementary to the nucleotide cassette.

229. The method of claim 228, wherein the nucleotide cassette is a ssDNA cassette, and wherein the method further comprises:
(I)
(e) contacting at least one copy of a nucleic acid identical or complementary to the nucleotide cassette with a composition comprising
(i) a guide nucleic acid comprising a nucleic acid sequence complementary to the nucleotide cassette;
(ii) a Cas enzyme with collateral cleavage activity; and
(iii) a nucleic acid reporter probe susceptible to the collateral cleavage activity, so that the nucleic acid reporter probe has a detectably different first uncleaved state and a second cleaved state; and
(f) detecting cleavage of the nucleic acid reporter probe by detecting a difference between the first state and the second state to determine the presence of the target nucleic acid sequence in the sample: or
(II)
(e) contacting at least one copy of the nucleic acid identical or complementary to the nucleotide cassette with a composition comprising
(i) a capture probe; and
(ii) a conjugate capture probe comprising a detectable label and a 3′ blocking molecule; and
(f) detecting presence of the detectable label bound to a solid substrate to determine the presence of the target nucleic acid sequence in the sample.

230. (canceled)

231. The composition according to claim 1, further comprising a forward primer that comprises:
(i) a 3′ nucleic acid sequence complementary to a target nucleic acid sequence downstream of or 5′ to the first or the second native restriction enzyme recognition sequence; and
(ii) a 5′ SDA primer binding sequence comprising a partial restriction enzyme recognition sequence.

232. The composition according claim 231, wherein the second SDA primer comprises:
(i) a sequence complementary to the forward primer 5′ SDA primer binding sequence comprising a partial restriction enzyme recognition sequence;
(ii) a 3′ blocking molecule; and
(iii) a 5′ stabilization sequence comprising at least 8 nucleotides, comprising a partial restriction enzyme recognition sequence that together with the partial restriction enzyme recognition sequence in the 5′ SDA primer binding sequence form a complete restriction enzyme recognition sequence.
